(19) United States
(12) Patent Application Publication
(10) Pub. No.: US 2005/0107316 A1
(43) Pub. Date: May 19, 2005

(54) Agent for inhibiting development or progress of proliferative diseases and especially cancer diseases and pharmaceutical composition containing said agent

(76) Inventors: Klaus Strebhardt, Frankfurt (DE); Birgit Spankuch-Schmitt, Frankfurt (DE); Yuan Juping, Bad Vilbel (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/505,482

(22) PCT Filed: Feb. 21, 2003
(86) PCT No.: PCT/EP03/01809

(30) Foreign Application Priority Data
Feb. 22, 2002 (EP) 02003982.2
May 17, 2002 (EP) 02011074.8
Nov. 8, 2002 (EP) 02025103.9

(51) Int. Cl. A61K 48/00; C07H 21/02

(52) U.S. Cl. 514/44; 536/23.1

(57) ABSTRACT

The present invention concerns inhibition of the activity of PLK1, which seems to be connected with cancer growth. Duplex RNAs antisense oligonucleotides and inhibitory peptides have been found to be useful in such inhibition, therefor they are claimed as ingredients fo pharmaceutical compositions for the treatment of proliferative diseases, preferably cancer of various types.

The present invention is concerned with an agent for inhibiting development or progress of proliferative diesease and especially cancer disease or other diseases, which are accompanied by elevated levels of polo like kinase PLK1, a pharmaceutical composition containing said agent as well as a method for treating patients suffering from said diseases.

Increasing knowledge about the genetic control of cellular proliferation provides the basis for the rational design of specific therapeutic strategies aimed at the regulation of proliferative disorders such as cancer. A key regulator for the mitotic progression in mammalian cells is the polo-like kinase (PLK1) which is structurally related to the polo gene product of Drosophila melanogaster, Cdc5p of Saccharomyces cerevisiae and plo 1 + of Schizosaccharomyces pombe (Glover et al. 1998). The PLKs from yeast, insects, amphibians and mammals represent a group of serine/threonine kinases that share a high degree of homology suggesting that the proteins have a close evolutionary and thereby functional relationship. The ability to regulate multiple stages of the mitotic progression is the hallmark of polo-like kinases (PLKs). Detailed information about the role of PLKs for mitotic progression came from genetic studies on Drosophila polo, the founding member of the family. Drosophila mutants homozygous for a strong mutant polo allele die as larvae (Llamazares et al., 1991; Sunkel et al., 1988). Flies with a weaker mutant allele produce embryos with severe mitotic defects such as condensed chromosomes with irregular microtubule arrays and a lack of organized centrosomes. Additional genetic approaches provided further insight into the function of the putative yeast homologues of polo. In Saccharomyces cerevisiae mutations in the CDC5 gene cause abnormalities in both mitotic and meiotic divisions (Byers et al., 1974; Hartwell et al., 1973; Sharon et al., 1990). CDC5 mutants arrest in late mitosis as large, budded cells with partially segregated nuclei on an elongated spindle (Kitada et al., 1993). Furthermore, loss of plo1⁺ function in Schizosaccharomyces pombe has two consequences: It leads to either a mitotic arrest in which condensed chromosomes are associated with a monopolar spindle or following the completion of nuclear division to a failure in septation (Ohkura et al., 1995). Taken together, deletion or strong mutations in plk-coding genes of different species cause severe growth retardation or even cellular lethality suggesting that PLKs play key roles for the mitotic progression of lower eukaryotes.

Whereas the yeasts can achieve cell-cycle progression utilizing a single cyclin-dependent kinase, mammals have evolved multiple forms of both Cdks and their activating Cdc25 phosphatases. The phylogenetic development of the plk family is comparable. In contrast to lower eukaryotes, which possess only one plk gene, at least three PLKs (PLK1, PLK2/SNK, PLK3/FNK) could be isolated from mammalian cells (Clay et al., 1993; Golsteyn et al., 1994; Holtrich et al., 1994; Holtrich et al., 2000; Lake et al., 1993; Li et al., 1996; Simmons et al., 1992). Their specific functions are very complex and in particular their ability to complement each other remains to be elucidated.

Increasing evidence supports the concept that PLKs regulate pivotal stages throughout mitosis including its initiation by activating Cdc2 through Cdc25 and direct phosphorylation of cyclin B1 targeting Cdc2/cyclin B1 to the nucleus. Furthermore, PLK1 contributes to centrosome maturation, bipolar spindle formation, DNA damage checkpoint adaptation and activation of Cdc16, Cdc27 as components of the anaphase-promoting complex (APC) for mitotic exit. Finally, PLKs are key regulators of cytokinesis (for review see Glover et al. 1998 and Nigg 1998).

Mammalian PLK1is overexpressed in rapidly proliferating cells and various human tumors (Yuan et al. 1997). An increasing body of evidence suggests that the frequency of PLK1 expression is of prognostic value for patients suffering from different types of tumors like non-small cell lung cancer, squamous cell carcinomas of head and neck, melanomas, oropharyngeal carcinomas, ovarian and endometrial carcinomas (Strebhardt 2001). Many data implicate that PLK1 participates in pathways, which override checkpoint arrests. PLK1 is needed for the Ca²⁺-induced release of Xenopus egg extracts from the meiotic M phase arrest (Descombes and Nigg 1998). Cdc5p is clearly required for adaptation to a DNA damage checkpoint in Saccharomyces cerevisiae (Toczyski et al. 1997). Moreover, expression of active PLK1 can override the G₂ arrest induced by DNA damage in mammalian cells (Smits et al. 2000). Thus, it is tempting to speculate that PLKs may play a role in overriding spindle and/or DNA damage checkpoints. Overexpression of PLK1 might be involved in malignant proliferation. Furthermore, constitutive expression of PLK1 in NIH-3T3 cells causes oncogenic focus formation and induces tumor growth in nude mice suggesting that PLK1 may contribute to cancer progression (Smith et al. 1997). Disrupting the function of PLKs could be an important application for cancer therapy.

Emerging from these results and speculations of the state of the art, it was an object of the present invention, to provide a possibility to inhibit or at least reduce the PLK1 activity and this way to allow for treatment of patients suffering from cancer diseases.

This object is solved by providing agents for inhibiting development or progress of proliferative diseases and especially cancer diseases, such agent being able to decrease or inhibit the activity of polo like kinase 1 (PLK1) in mammalian cells.

The merits of the present invention lies in the fact, that it has been found that inhibiting PLK1 activity actually can have very promising effects on patient suffering from proliferative diseases. It has further been found that there are no or only comparably little adverse effects to be expected on other cells of the body.

There a several possiblities to inhibit or reduce PLK1 activity. Such possibilities especially include administration of substances that interfere with PLK1 activity as well as substances that interfere with formation of PLK1 in the cells. All such possiblities are considered to be encompassed by the present invention as long as a sufficient inhibitory effect can be observed.

In a first preferred aspect of the present invention, an agent of the invention contains as active agent at least one short interfering RNA (siRNA) which is directed against the PLK1 gene and results in formation of double-stranded RNA and subsequent degradation of the PLK1 mRNA.

In eukaryotes, double-stranded (ds) RNA induces sequence-specific inhibition of gene expression referred to as RNA interference (RNAi). Since PLK1 (polo-like kinase 1) expression is elevated in a broad range of human tumors, in the framework of the present invention RNAi was exploited to define the role of PLK1 for neoplastic proliferation and to impair PLK1 activity.

RNA-interference (RNAi) as part of a primitive immune system represents the ability of some viruses, transgenes or RNAs to trigger post-transcriptional degradation of homologous cellular RNAs (Sharp, 2001; Fire, 1999). This mechanism has evolved to protect the genome of an organism against the hostile environment with dangerous opportunities for unwanted gene expression and with parasites (transposons and viruses). Double-stranded RNA (dsRNA) has been shown to trigger sequence-specific gene silencing in numerous organisms such as nematodes, plants, trypanosomes, fruit flies and planaria. Studies in C. elegans and Drosophila revealed that a few molecules per cell are sufficient to eliminate a much larger pool of endogenous mRNA and thereby induce a strong RNAi response suggesting a catalytic or amplification mechanism to contribute to gene silencing (Fire et al. 1998, Montgomery et al., 1998). After selection of a specific mRNA as target for RNAi, endogenous cleavage is a key step in degradation of the target mRNA. In Drosophila RNAi is independent of mRNA translation but requires ATP (Zamore, 2000). During this process siRNA is reduced in size to fragments of 21-23 nucleotides by a ribonuclease III protein that is independent of the targeted mRNA. Subsequent cleavage of the mRNA was observed only within the region of identity with the siRNA. In a recent study RNA duplexes of 21 nucleotides in length were shown to suppress gene expression in mammalian cell lines (Elbashir et al., 2001). This report suggested that longer dsRNAs (50- and 500-bp) induced nonspecific reduction in reporter-gene expression probably as part of an interferon response (Der et al., 1997).

After transfection Northern and Western blot analyses were used to examine the potential of 21-nt-long, si RNAs targeted against human PLK1 for specific inhibition of PLK1 gene expression. Furthermore, the influence of siRNAs on the phenotype and the proliferation of cancer cells and of primary cells was monitored.

Treatment of cancer cell lines (MCF-7 breast, HeLa S3 cervix, SW-480 colon and A549 lung) with appropriate siRNAs resulted in a sequence-specific decrease in the level of mRNA and protein expressed from the human PLK1-gene. The analysis of mitotically arrested SW-480 cells by fluorescence microscopy revealed centrosomes that lost their ability for microtubule nucleation. Moreover, siRNA treatment against PLK1 resulted in a potent antiproliferative effect and apoptosis in tumor cells of different origin consistent with a powerful RNA silencing effect for the applied siRNAs. In contrast, primary mammary epithelial cells exhibited low sensitivity towards PLK1-specific dsRNA. Statistical tests were performed using two way ANOVA (Analysis of Variance).

These data indicate that PLK1 function is essential for centrosome-mediated microtubule events and consequently for spindle assembly. The observations further implicate that siRNAs targeted against human PLK1 may be valuable tools as antiproliferative agents that display activity against a broad spectrum of neoplastic cells at very low doses.

According to the invention, therefore, one solution for the object of the present invention is providing agents that contain siRNA which are targeted against the PLK1 gene for inhibiting or reducing PLK1 activity.

Using this first approach of the present invention it could be demonstrated that administration of siRNAs targeted against human PLK1 reduced the level of PLK1 transcripts in cell culture efficiently. Also antisense RNA can be used to interfere with PLK1 expression efficiently.

RNA interference has become a powerful tool for the analysis of gene function in invertebrates and plants (Sharp, 2001). In mammalian cells, dsRNA is processed into siRNAs (Elbashir et al., 2001; Billy et al., 2001; Paddison et al., 2002), but nonspecific responses occur if the dsRNA molecules are longer than about 30 nt. Tuschl and colleagues made the interesting observation that transfection of synthetic 21-nt-RNA duplexes into mammalian cells reduces endogenous mRNA levels in a sequence-specific manner (Harborth et al., 2001; Hutvagner et al., 2001). The present study demonstrates that administration of siRNAs targeted against human PLK1 reduces the level of PLK1 transcripts in cell culture. siRNA4 exhibits a pronounced inhibitory effect at a concentration of 5.6 nM in different cancer cell lines. The effect disappears if the siRNA concentration is reduced below 0.5 nM. One of the most attractive features of siRNA-based gene silencing is the potent inhibitory effect at low concentrations. In comparison, phosphorothioate antisense oligonucleotides display IC50 values between 100 and 500 nM (Tamm et al., 2001; Spänkuch-Schmitt et al., 2002). Thus, possible limitations of phosphorothioate antisense oligonucleotides as pharmacological agent due to the potential toxicity seem to be less likely in the case of siRNA, because efficient knock-down of target genes can be achieved with very low concentrations. Moreover, considerable attention was paid to the specificity of administered siRNAs. First, within a small set of tested siRNAs targeted to different regions of human PLK1 only certain candidates had a potent silencing effect. The variations seen in the effectiveness of RNAi in a particular cell line could be influenced by the ability of a particular cell-type to form an RNAi silencing complex. Second, a scrambled siRNA4S (permutated sequence of siRNA4) or a siRNA targeted to lamin had only little effect on the level of PLK1 mRNA. Four tumor cell types were responsive to the antiproliferative effects of siRNA4 (MCF-7 breast, HeLa S3 cervix, SW-480 colon and A549 lung), supporting the premise that PLK1 silencing will be useful for the treatment of tumors. This view gained attractive support by the observation that primary epithelial cells were not suppressed by siRNA at concentrations that extinguished tumor cells. A low transfection efficiency of primary cells could be the reason for their reduced sensitivity towards siRNAs. Thus, toxic side-effects in normal cells exerted by siRNA targeted to human PLK1 are less likely. Taken together, the present study seems to indicate that the inhibitory effect of siRNA4 on PLK1 expression and the biological consequences that appear to result from these inhibitory effects in cell culture occur through an RNA silencing mechanism.

In previous studies an adenoviral delivery of dominant negative forms led to the inhibition of PLK1 function (Cogswell et al., 2000). However, the treatment of cancer patients with recombinant adenoviral vectors has still considerable limitations (Vorburger and Hunt, 2002). Today's second and third generation adenoviral vectors have decreased toxicity and result in prolonged gene expression in vivo (Schiedner et al., 1998). Nevertheless, an important limitation in the use of recombinant adenovirus has been the difficulty in obtaining efficient gene transfer upon a second administration of virus due to formation of neutralizing antibodies. The ability to use siRNA to target selectively genetic mechanisms involved in tumorigenesis gives rise to the fascinating chance that these novel agents could be used, not only as a new class of chemotherapeutic agents for the systemic treatment of cancer patients, but also to gain a better understanding of the critical molecular events responsible for initiating and maintaining the cancer phenotype. The above mentioned results raise some intriguing questions relative to the role of PLK1 in cancer cells. Centrosomes play a critical role in generating genetic instability in cancer cells (Brinkley, 2001; Doxsey, 2001). They contribute to spindle abnormalities and disturbed chromosome segregation, which is often accompanied with profound alterations in key cellular functions like apoptosis, cell cycle progression, control of cell cycle checkpoints and cell growth regulation. Different studies tested the impact of PLK1 on the function of mammalian centrosomes (Lane and Nigg, 1996; Cogswell et al., 2001; Doxsey, 2001; do Carmo, 2001). While the analysis of HeLa cells microinjected with PLK1-specific antibodies revealed monoastral microtubule arrays that were nucleated from duplicated but unseparated centrosomes, RNA silencing allowed to separate centrosome division from microtubule anchoring: Centrosomes still divided and separated from each other but obviously without microtubule interaction. If the pericentriolar matrix surrounding centrioles becomes dissolved in early prophase, centrosomes are not kept in close proximity any longer. Lack of PLK1 after siRNA treatment prevents the formation of the microtubule nucleation complex required for aster and spindle formation. Thus, knock-down of PLK1 function may induce different mitotic phenotypes at various stages due to varying checkpoint controls in different cancer cells or might alternatively correlate to the endogenous level of PLK1 mRNA in tumor cells reflecting a mutation-sensitive interaction of PLK1 mRNA and Hsp90 (Simizu and Osada, 2000).

Extra centrosomes in cancer cells might lead to chromosome missorting and damage causing aneuploidy which may induce the loss of tumor suppressor genes or activate oncogenes. Recent observations pointed out that centrosomal abnormalities can be detected in early forms of human breast and prostate cancer (Pihan et al., 2001). This might suggest that centrosomes are the driving force of cancer formation instead of being a consequence of it. The cell culture experiments presented herein indicate that although the mammalian genome contains several polo-related kinases, the knock-down of PLK1 alone by siRNA-mediated RNA silencing is sufficient to prevent centrosome-induced spindle formation and to induce apoptosis in different tumor cells.

Further preferred embodiments of this first aspect of the present invention include agents containing a siRNA or antisense RNA of 15 to 30 nucleotides. Although the interfering RNA can be derived from any part of the mRNA, including open reading frame as well as 3′ and/or 5′ untranslated regions, it is preferred that the sequence of such RNA corresponds to nucleotide stretches of the PLK1 gene which are located between nucleotides 170 and 1600 of the PLK1 sequence (NCBI accession number: X75932).

Data about RNA interference itself and about structural modifications and effects on the effectiveness of SiRNAs are described by Hannon et al. and Elbashir et al. (Hannon et al. 2002; Elbashir et al. 2001b).

In the context of the present invention siRNAs 2 to 5 are especially preferred tools for inhibiting PLK1 activity: siRNA2 corresponds to positions 178-200, siRNA3 to 362-384, siRNA4 to 1416-1438 and siRNA5 to 1570-1592 of the PLK1 gene.

These siRNAs show especially potent inhibition of the PLK1 activity and their use for treating proliferative diseases and especially cancer diseases is accordingly especially preferred.

It is also possible within the framework of the present invention to use mutated or chemically modified RNAs, especially modified siRNAs, to achieve a prolongation of the half-life time of the siRNA in the patient. Correspondingly modified RNAs are described for example by Amarzguioui et al. (2003), Chiu and Rana (2002), Elbashir et al. (2001) and Nykanen et al. (2001).

The application of siRNA to a patient can be effected via any suitable route, especially intravenous injection of siRNA solutions (preferably as pure as possible), intratumoral injections of siRNA (preferably pure) as well as i.m. or subcutaneous injections are preferred routes. Application can also be affected in form of a spray, especially when indications like a lung carcinoma or lung cancer metastasis are concerned, application in form of an ointment or a cream for dermal or transdermal applications like treating melanomas or skin metastases; also suitable are combined injections of siRNA with reagents for transfection to promote or guarantee uptake of the siRNA into cells, and/or combination with RNase inhibitors, e.g. to inactivate RNases (applicable for all routes of administration). Another route of administration is oral uptake, e.g. in form of a yoghurt or hard-cheese with transfected lactobacilli (e.g. lactobacillus acidophilus), streptococci, or lactococci propionibacteria, such bacteria producing siRNA. Transfection of such bacteria with vectors or genes which lead to siRNA production can be effected according to methods known in the art. Such bacteria can produce siRNA either in the food stuff they are contained in, or in the patient.

An oral administration can also be effective in any form of tablet, capsule, powder or in liquid form. Especially preferred is a pharmaceutical agent containing an siRNA in form of a gelatine capsule leading to an uptake of siRNA in the stomach. A special form of treating persons which corresponds to a prophylaxis is an application of siRNA (either pure or with a transfection reagent) to patients who have a predisposition, e.g. a genetic and/or environmental predisposition for a particular cancer disease, or just to healthy persons as a genoprevention (as opposed to chemoprevention) against cancer diseases. In the framework of the present invention, a prophylactic application to a patient can substitute surgical prevention that is presently used like for example ovarectomy or a tubal sterilization.

As a dosage for the application of siRNA an amount of 0.1 to 3.3 mg/kg body weight of the patient is especially preferred. This dosage is especially preferably used for intravenous injection.

For oral application in the form of a yoghurt or a cheese containing e.g. lactobacilli that produce siRNA, the concentration of the bacteria should be sufficient to produce amounts of siRNA in such food stuffs that are suitable for average adult persons. Since application of such siRNA does not lead to adverse effects, a prophylactic conception of food stuff producing such siRNAs could lead to a considerable reduction of proliferative diseases without endangering consumers otherwise.

Besides application of siRNAs to induce degradation of mRNA, duplex RNAs can also be used to induce secondary modifications (methylation) of for example promoter sequences (Mette et al., 2000). Using this effect, the PLK1 gene is inactivated on transcriptional level, i.e. the formation of PLK1 mRNA is reduced or prevented.

A further preferred embodiment of the present invention is an agent according to the present invention for suppressing expression of the PLK1 gene which comprises an effective amount of
1) an RNA expression system and optionally
2) a nuclease inhibiting substance, wherein said RNA expresssion system contains
a) at least one RNA polymerase specific promoter sequence and under the transcriptional control of said promoter sequence
b) at least one genetic information homologous to the PLK1 gene, such genetic information being transcribed under suitable conditions and in the presence of an RNA polymerase into short interfering RNA, preferably small interfering double stranded hairpin RNA or short RNA antisense strands (20 to 25 nt in length).

In the framework of the investigations leading to the present invention it was surprisingly found that an effective and persistent gene silencing is possible using the RNA expression system as described above. It has been found that when using certain kinds of expression vectors, like U6- and H1-vectors, a potent suppression of expression of the PLK1 gene can be achieved. Adding a nuclease inhibiting substance can intensify such suppression. For other vector systems it might be advisable to use a combination of RNA expression system and nuclease inhibiting substance to achieve the desired suppression of expression. This nuclease inhibiting substances avoid the breakdown and removal of the expression vector containing the genetic information for the RNA and possibly also the RNA itself. Thus the expression vector in the presence of RNA polymerases which are abundant in vivo, can constitutively express RNAs targeted against the gene to be silenced for a sufficiently long time. It was found that application of the pharmaceutical composition of the present invention every other day was sufficient to suppress tumor growth and to allow immune systems to attack the tumors and thereby even reduce tumour size.

The pharmaceutical composition of the present invention further showed no marked detrimental side effects to the treated person. To the contrary, the application can take place easily by for example intravenous injection of the composition.

The composition according to the invention contains an RNA expression system that either encodes siRNA, preferably small interfering hairpin RNA, or short antisense RNA, both of which are homologous to the gene to be expressed and interfere with proper transcription of said gene which leads to silencing of the targeted gene. The RNA expression system can contain one promoter and genetic information for one kind of interfering RNA, however, it is also possible that the composition contains an expression system containing genetic information for more than one kind of interfering RNA. In such case this genetic information for more than one kind of RNA can be expressed under the control of one promoter but also several promoters, being the same or different, can be used. The pharmaceutical composition of the present invention can also contain more than one expression system, each leading to formation of one or more kinds of interfering RNA.

With the present invention a powerful novel strategy is provided to suppress very efficiently tumor cell proliferation in cell culture and in vivo. For the first time it could be demonstrated that U6 promoter-driven hairpin RNAs targeted against PLK1 stabilized by the nuclease inhibitor (aurin tricarboxylic acid) ATA suppress tumor growth in nude mice when administered every other day systemically by intravenous injection. The power to encode a long-lasting silencing signal allows the combination of hairpin-mediated silencing with in vivo and gene delivery strategies for therapeutic approaches based on stable RNA interference in humans. Systemic RNA silencing in patients provides the fascinating perspective of using therapeutics that are of natural composition compared to chemical compounds, that are highly specific and that are of cost effective manufacturing.

In a preferred embodiment of the present invention, the RNA expression system contains a promoter sequence which is specific for class III RNA polymerases and especially preferably it contains the U6 promoter (Sui et al. 2002). Using the U6 promoter in an expression system leads to a very stable expression of the corresponding siRNA or short RNA antisense strands and it is conceivable that nuclease inhibitor concentrations can even be lowered and possibly even nuclease inhibitors are not necessary at all for obtaining sufficient interfering RNA expression and corresponding gene silencing when using the U6 promoter.

The basic structure of the expression system is not critical as long as it allows for efficient transcription of the genetic information of component b) of the expression system. The expression can occur either con stitutively or inducably. A constitutive expression is preferred in the present context. Usually a bacterial plasmid or a viral vector will form the basis of the expression system, however, the present invention is not limited thereto.

The preconditions for formation of siRNA are known to the person skilled in the art and can also be inferred from the references mentioned supra. The expression system contains two complementary and inverted DNA sequences which upon transcription by RNA polymerase lead to formation of double stranded RNA products. Such RNA products preferably are 15 to 30 nucleotides long and are homologous to the PLK1 gene.

In a especially preferred embodiment of the present invention, the DNA coding for the siRNA is contained on a vector in the form of two complementary and inverted sequences which are adjacent to each other but divided by a spacer sequence, such spacer sequence being preferably 3 to 10 nucleotides long. Upon transcription of the expression system a small hairpin double stranded RNA (shRNA) is formed which interferes with expression of the PLK1 gene. It is however also possible to provide for separate transcription of the two strands of the siRNA which will anneal after transcription automatically or of short RNA antisense strands.

In a preferred expression system on the 3′ end of the sequences to be transcribed there is a RNA polymerase stop signal, preferably a T multimer.

As nuclease inhibitor every physiologically acceptable substance can be used that inhibits or decreases degradation of the expression system for a significant time. In a preferred embodiment of the present invention, as nuclease inhibitor aurin tricarboxylic acid (ATA) is used. However, the invention is not limited to this substance and every other nuclease inhibiting substance showing substantially similiar properties related to nuclease inhibiton and physiological tolerance is applicable.

It is a very favorable characterisitic of the agent of the present invention that it can be applied easily, preferably by intravenous injection. In this context it is further preferred to include the agent in a physiologically acceptable solution, e.g. phosphate buffered saline. Administration of larger volumes of solution promote the immediate effectiveness of gene suppression by the present pharmaceutical composition. Suitable amounts of administration of the expression system depend on the size of the plasmid or vector used. However, a preferred dosage of expression system including the sequences being transcribed to siRNAs lies between 0.05 to 0.5 mg/kg body weight of the patient. The effect of the generated siRNA can easily be monitored by assaying for remaining expression of the PLK1 gene. Thus the dosage can easily be adjusted to the needs. Also the addition of further substances into the injection solution is possible. Such further substances can include symptom alleviating substances, substances that strengthen the patient, antibiotics or other suitable substances. Also substances that facilitate application and carriers or auxiliaries can be included in the formulation.

An oral application of the agent according to the invention, preferably using nanoparticles as administration vehicles, is also possible.

In preferred embodiments of the present invention, the agent is used for tumor therapy or prophylaxis. As described above and in more detail in the following examples, suppression of PLK1 expression has been shown to be an efficient method for treatment of cancer disease of several tissues.

In a second preferred aspect of the present invention, the object of inhibiting or decreasing PLK1 activity is solved by providing phosphorothioate antisense oligonucleotides (ASOs) which are directed against the PLK1 gene and using such ASOs as an agent according to the present invention.

As already mentioned above, a central role for polo-like kinases (PLK) in regulating several stages of mitotic progression has been born out in several species. Overexpression of PLK1 is observed in the majority of hitherto analyzed human tumors. PLK1 overexpression is a negative prognostic factor in patients suffering from non-small cell lung cancer, head and neck tumors, esophageal carcinomas and melanomas. In order to define the role of PLK1 for mitotic progression of human cells and for neoplastic cell growth, phosphorothioate antisense oligonucleotides (ASOs) were tested to selectively downregulate PLK1 expression in MDA-MB-435 (breast cancer), HeLa S3 (cervical carcinoma) and A549 (non-small cell lung cancer) cells. ASOs were identified which suppress PLK1 mRNA and protein in a dose-dependent and sequence-specific manner. This approach also led to reduced PLK1 serine/threonine kinase activity. Downregulation of cellular PLK1 levels in cancer cells altered cell cycle progression moderately with an elevated percentage (20-30%) of cells in G₂/M. Furthermore, cells with reduced PLK1 protein gained a rounded phenotype with multiple centrosomes. Moreover, ASO treatment resulted in potent antiproliferative effects in cell culture. Considerable antitumor activity was observed in vivo against A549 cells. This study suggests that antisense inhibitors targeted against PLK1 at well tolerated doses are to be considered as a cancer therapeutic agent.

Increasing knowledge about the genetic control of cellular proliferation provides the basis for the rational design of specific therapeutic strategies aimed at the regulation of proliferative disorders such as cancer. Although certain unanswered questions concerning the applicability of the antisense technology remain (Stein, 1995; Wagner, 1995), this experimental approach can, when targeted to key elements of proliferation-relevant signaltransduction pathways, prevent the development of specific human cancers. Several phosphorothioate antisense oligonucleotides (ASOs) are currently being evaluated in patients suffering from different types of cancer such as ovarian, colon, lymphoma and melanomas (Crooke, 2000).

Furthermore, studies in which these drugs are used in combination with traditional chemotherapeutic agents are in progress.

To elucidate the role of PLK1 for the inhibition of tumor cell growth, in the present study the potential of phosphorothioate ASOs targeted against human PLK1 to inhibit its mRNA and protein expression was tested. Then the effects of PLK1-specific ASOs on the proliferative activity of human tumor cells (breast cancer, MDA-MB-435; non-small cell lung cancer, A549 and cervical carcinoma, HeLa S3) in vitro and in vivo were evaluated to shed light on the role of PLK1 as target for cancer treatment.

The work leading to the present invention therefore also provides evidence for phosphorothioate ASOs targeted against PLK1 and their use for treatment of proliferative diseases like for example cancer diseases. Agents containing such phosphorothioate ASOs as well as their uses are further subject matters of the present invention.

In the context of the present invention it is further preferred to use ASOs containing about 15 to 30 nucleotides. Although it is preferred that such ASOs are homologous to the 3′ untranslated region of the PLK1 gene ASOs which are derived from any part of the PLK1 gene can be used, including open reading frame and 5′ and 3′ untranslated regions. The most preferred ASOs according to the invention are P12 and P13 as herein described.

Chemically modified ASOs like for example described in Agrawal et al. (1997), Braasch and Corey (2002) or Krieg et al. (1996) can also be used and are often preferred because of their prolonged half life time in patients. Possible modifications include mixed backbone oligonucleotides or morpholino oligonucleotides.

Dosages of ASOs for therapy or prophylaxis preferably are between 0.1 and 10 mg/kg body weight of a patient per day. ASOs can also be applied in form of a combination with common chemotherapeutica like Doxorubicin, 5-fluorouracil or Leucovorin or in combination with monoclonal antibodies like e.g. Herceptin. For individual applications, the dosage can also be higher like up to 50 mg/kg body weight of the patient or more. The administration routes can be as described for siRNA.

Yet a further preferred aspect and embodiment of the present invention is the use of inhibitory peptides as active substance in the agent for the inhibition of proliferative diseases and especially cancer diseases.

From the view of the primary structure, PLKs contain a strikingly conserved sequence within their C-terminal domain, termed the polo-box, which is 30 amino acids in length. Without impairing kinase activity, three mutations in the polo-box of PLK1 abolish its ability to functionally complement the defect associated with a Cdc5-1 temperature-sensitive mutation (Lee et al. 1996). Recent studies revealed that fission yeast Plo 1 interacts with the APC through the polo-box and the tetratricopeptide repeat domain of the subunit, Cut23 (May et al. 2002). A mutation in Cut23, which specifically disrupts the interaction with the polo-box, results in metaphase arrest. Taken together, the data suggest that the polo-box of PLKs plays a critical role for the function of PLK1, in particular for its spatial distribution and for the physical interaction with substrates.

Due to the rapid development of technical protein synthesis and the advantage of non-gene interference, the treatment with peptides is becoming a powerful new approach for tumor therapy (Latham 1999). A 16-mer peptide, derived from the homeodomain of Antennapedia, has been reported to enter cells readily via a non-endocytotic and receptor- and transporter-independent pathway (Derossi et al. 1996). In the studies leading to the present invention, this Antennapedia-peptide was linked to the wild-type polo-box or to a mutated polo-box and its impact on the proliferation of cancer cells was analyzed.

PLK1 plays various critical roles in the passage of cells through M phase. It is overexpressed in rapidly proliferating cells and tumors (Yuan et al. 1997 and Strebhardt 2001). Data implicate that PLK1 contributes to override spindle- and DNA damage-checkpoints (Descombes et al. 1998, Toczyski et al. 1997 and Smits et al. 2000), which makes PLK1 an attractive target for cancer therapy. Recently, it was reported that overexpression of the C-terminal domain of PLK1 is more efficient in causing mitotic delay or arrest than wild-type or kinase-defective PLK1 (Jang et al. 2002). This observation is due to the binding of the C-terminus to full-length PLK1 or to the catalytic domain of PLK1, which causes the inhibition of its kinase activity (Jang et al. 2002). The region within the C-terminal domain mediating this effect is unknown. The polo-box represents a highly conserved sequence within the C-terminal noncatalytic region of the Plk-family and has not been observed in proteins other than PLKs yet. The exchange of three amino acids within the polo-box abolished the proper localization of PLK1 and disrupted its kinase function (Lee et al. 1999). In the study leading to the present invention it could be demonstrated for the first time that a fusion protein containing the polo-box of PLK1 and a transmembrane carrier from Antennapedia used for the treatment of cancer cells is a novel strategy to inhibit the function of PLK1. It was revealed that the polo-box inhibits the proliferation of various cancer cell lines by inducing apoptosis. Strong effects were observed in MCF-7 cells which is possibly connected to the functional integrity of wild-type p53 and Rb, two tumorsuppressor proteins leading to better apoptotic reaction. The inhibitory effect began after 10 h of treatment and reached its apoptotic peak at 24 h. The typical morphology of apoptotic cells was observed in all three cancer lines. As reported for microinjecting of PLK1-antibodies or for expression of a dominant-negative form of PLK1 (Lane et al. 1996, Cogswell et al. 2000), polo-box peptide induced also mitotic arrest. The FACS-analysis documented an increase of the G₂/M-population and in particular a 3-4 fold increase of mitotic cells in polo-box-treated cells.

In many treated cells chromosomes appeared to be randomly distributed and improperly condensed. Multiple or monoastral spindle poles were observed which is in line with observations in cells transfected with C-terminal domain of PLK1 (Jang et al. 2002). In addition, polo-box-treated cells displayed daughter cells still connected by strings of cytoplasm. Since P3-treatment did not induce incomplete separation of arising cells, polo-box-mediated functions seemed also to be involved in cytokinesis (data not shown).

Major abnormalities in cancer cells including the inhibitory effect on proliferation were induced only by the wild-type polo-box, but not by the mutated form P2. Different mechanisms could contribute to the apoptotic impact exerted by polo-box peptides. At first, kinase assays revealed an inhibitory effect on substrate phosphorylation by PLK1: The polo-box peptide could prevent the binding of PLK1 to its substrate thereby acting in a competitive manner. Secondly, recent evidence documents that the C-terminal domain of PLK1 can bind to full-length or the catalytic domain of PLK1 (Jang et al., 2002). This interaction is interrupted when Thr-210 is substituted with an aspartatic residue. In addition, the function of PLK3 was also shown to depend on its C-terminal domain (Conn et al. 2000). Still, the region within the C-terminal domains of PLK1 and PLK3 responsible for regulating the kinase activity remains to be elucidated. It is intriguing to consider the polo-box, a domain very well conserved during evolution, as candidate for this regulatory function possibly by binding to a region surrounding Thr-210 in PLK1. This hypothesis gains further support from previous observations which demonstrated that mutations in the polo-box reduce the kinase activity of PLK1 (Lee et al. 1999). Future investigations are required to study the polo-box as structural component for an intramolecular modulation of the activity of PLKs.

Whereas in yeasts and Drosophila only a single Plk has been identified to date, the genome of higher vertebrates encompasses at least three PLKs. The remaining two family members, PLK2 (Snk) and PLK3 (Fnk/Prk) belong to the category of immediate-early response genes (Glover et al. 1998 and Nigg et al. 1998). Functional assays imply that PLK1 and Plk3 are likely to have both overlapping and unique functions within the cell cycle (Glover et al. 1998). Plk3 links DNA damage functionally to cell cycle arrest and apoptosis partially via the p53 pathway (Xie et al. 2001). Overexpression of Plk3 induces incomplete cytokinesis and apoptosis (Conn et al. 2000). Considering the high homology (74%) of the polo-boxes belonging to PLK1-3, it could not be excluded that the polo-box-specific peptide (P1) derived from PLK1 may also inhibit at least partially the function of Plk2 and Plk3, which might contribute to the effects observed in our study. Especially inhibition of Plk3 might be involved in inducing apoptosis and incomplete cytokinesis. Taken together, polo-box-specific peptides inhibit proliferation of tumor cell lines by inducing mitotic arrest and apoptosis. In line with the rapid development of peptide synthesis, polo-box could be a powerful inhibitor for proliferation. Up to date PLK1-function was inhibited by expression of dominant-negative forms or by application of antibodies (Lane et al. 1996 and Cogswell et al. 2000). Beyond perspectives offered by these techniques the use of fusion peptides such as P1 could open new ways for the systemic treatment of animals with localized tumors or even with disseminated disease.

In a preferred embodiment of this third aspect of the present invention the agent contains an inhibitory peptide which comprises 3 to 50 and most preferably 10 to 30 amino acids. It is especially preferred that the peptide correponds in sequence to a wild type (aa 410-439 in PLK1) or a mutated polo box or its polo-box similiar structures in PLK1 -3. It is further preferred to use fusion peptides wherein the inhibitory peptide is linked to a carrier peptide, like for example an Antennapedia carrier peptide.

Especially preferred mutated polo box peptides can contain the following modifications: L-forward, L-reverse, D-reverse (retro-inverso), side-chain and back bone modification, cyclic form and repeat (tandem), as well as other modifications.

Routes of administration can be as described above for siRNA and ASOs, a prefered dosage is about 1-100 mg/kg body weight of the patient, preferably an application takes place one to five times per week for a duration of 1 to 8 weeks. Especially preferred is an intratumoral injection of an inhibitory peptide with a dosage of 15-25 mg/kg, three times per week for a duration of 4-6 weeks.

The peptide can also be linked to a protein transduction domain which enables the peptide to enter cells. Another similiar possibility is combination of the peptide with a transduction domain without need for a chemical covalent coupling as for example described by Morris et al. (2001).

Especially preferred peptides that can be used in agents of this invention are the peptides P1 and P2 as herein described.

The agents according to the present invention, and especially the three preferred agents containing either siRNA, ASOs or inhibitory peptides have been shown to be promising and powerful tools for inhibiting proliferative disease, while not affecting normal and healthy cells and organisms.

Accordingly, a further subject of the present invention is a pharmaceutical composition containing an agent as defined above and preferably containing at least one of an siRNA, ASO and inhibitory peptide with properties as described in connection with the agent.

A still further subject of the present invention is a method for treating patients suffering from proliferative disease and especially from cancer disease by administering an effective amount of an agent as defined above or a corresponding pharmaceutical composition.

Amounts that are effective can easily be determined by comparison of in vitro studies to effects obtained in vivo. Typically, amounts used are described above.

The route of administration of the agents or pharmaceutical composition is not critical and depends mostly on the kind of disease or tumour to be treated. The administration can take place for example orally, parenterally, intravenously or by direct injection or application to tumour tissue. For each form of application different carrier and auxilliary substances may be useful. Such auxiliary substances are well known to the man in the art and can be selected according to the needs. Also carrier substances can be useful in the pharmaceutical composition of the invention. Substances like liposomes and nanospheres can be used as carrier molecules, slow release materials into which agents according to the invention are included can be used for implantation at the tumour site or for other application forms. It is also possible to combine the agents according to the present invention with substances with instant symptomatic usefulness, like anaesthecis, antiinflammatory agents and the like. Also the combined application of the agents according to the invention with antibiotics, antifungal agents and the like might be beneficial and is encompassed within the scope of the present invention.

The investigational work shown in the examples as well as the results deduced therefrom together with the figures are meant to further illustrate the present invention.

### FIGURE LEGENDS

FIG. 1. Reduction of PLK1 mRNA in cancer cells and HMECs. (A) Northern blot analysis of PLK1 mRNA in MCF-7 cells 6, 24 and 48 hrs after siRNA treatment (56 nM). To ascertain uniformity of loading membranes were reprobed with human β-actin. PLK1 and actin signals are indicated. Percent inhibition was calculated by comparison with PLK1 mRNA levels of cells treated with siRNA4S. Scatterplots represent three independent experiments, their means (indicated as horizontal bar) and 95% confidence intervals. (B) PLK1 mRNA expression in HeLa S3, (C) SW-480 and (D) A549 cells 6, 24 and 48 hrs after single siRNA application. (E) In HMECs PLK1 mRNA reduction was measured 48 hrs after treatment with siRNAs (2 μM).

FIG. 2. Inhibition of PLK1 protein expression by PLK1-specific siRNAs. Percent inhibition was calculated by comparison with PLK1 protein levels in cells treated with siRNA4S after standardization to actin levels. (A) Western blot analysis of PLK1 protein in MCF-7 cells 48 hrs after siRNA treatment (56 nM). Indicated are the signals for PLK1 and actin. Actin served as control for equal loading (upper panel). Scatterplots represent three independent experiments, their means (indicated as horizontal bar) and the 95% confidence intervals (lower panel). (B) Quantification of PLK1 protein in HeLa S3, (C) SW-480 and (D) A549 cells after normalization to actin, shown as scatterplots representing three independent experiments, means (indicated as horizontal bar) and standard deviations. (E) Western blot analysis of PLK1 protein 48 hrs after siRNA treatment depends on the concentration (0.5-566 nM) of siRNA4 in MCF-7 cells. (F) Kinase activity of immunoprecipitated endogenous PLK1 was reduced 48 hrs after siRNA4 treatment (56 nM) compared to control or siRNA4S-treated cells (upper panel). Coomassie staining served as control for equal loading of substrate (middle panel). Equal amounts of PLK1 were subjected to kinase assays (lower panel).

FIG. 3. Knock-down of lamin protein in MCF-7 cells and HMECs. (A) In MCF-7 cells 56 nM of siRNA1 were necessary for efficient reduction of lamin protein. (B) In HMECs 2 μM of siRNA1 were required to achieve a similar reduction. Lamin signals were normalized to actin and compared to control cells.

FIG. 4. siRNA4 treatment resulted in abrogated spindle formation at centrosomes in SW-480 cells and disturbed chromosome phenotypes in early mitosis of MCF-7 cells. Cells were transfected with siRNA4 (56 nM) and immunostained 48 hrs post transfection for γ-tubulin (green), α-tubulin (red) and DAPI (blue). (A) siRNA4-treated SW-480 cells with missing spindles at centrosomes (upper panel): Arrows point to a cell with centrosomes devoid of any microtubule connection. Untreated cells show normal spindle formation (lower panel). (B) Supernatant of MCF-7 cell cultures treated with siRNA4 contained many mitotic cells. Most of them were characterized by highly condensed, knob like chromosomes. Some chromosomes were located adjacent to the nucleus as indicated by arrows. (C) siRNA4-treated HMECs (2 μM) displayed centrosomes which were able to organize microtubules (upper panel). These cells proceeded through mitosis with normal phenotype (middle panel). Phenotypes of control cells and siRNA4-treated cells were similar (lower panel). (D) PLK1 staining (red) and α-tubulin staining (green) in control and siRNA4-treated MCF-7 cells (lower panel, 56 nM) and HMECs (upper panel, 2 μM). Both cell types showed reduction of PLK1 protein. Untreated MCF-7 cells had normal mitotic phenotypes. In contrast, siRNA4-treated MCF-7 cells showed impaired mitoses. In HMECs no mitoses could be found and therefore PLK1 reduction by siRNA4 in interphase cells is shown.

FIG. 5. PLK1-specific siRNA treatment induced G₂/M cell cycle arrest and apoptosis in cancer cells in contrast to HMECs. (A) FACScan analysis indicates G2/M arrest induced by siRNA4 in SW-480, MCF-7, HeLa S3 and A549 cells (56 nM) and in HMECs (2 μM) (left panel). Effects of siRNAs1-5 and siRNA4S on cell cycle distribution in cancer cell lines and HMECs (right panel). (B) DAPI staining after siRNA4 treatment of HMECs (upper panel, 2 μM) and MCF-7 cells (lower panel, 56 nM) to detect apoptotic phenotypes indicated by arrows. (C) CLSM analysis revealed apoptotic cells with disintegrated nuclear membranes and condensed chromatin (phase contrast image of MCF-7 cell). White arrows: apoptotic nuclei; black arrows: normal nuclei.

FIG. 6. Antiproliferative effects of PLK1-specific siRNA-mediated inhibition in cancer cells. siRNA against lamin served as internal negative control. Percent inhibition was calculated by comparison to the number of cells after 96 hrs grown in the absence of siRNAs. (A) Growth inhibition of MCF-7 cells over a period of 4 days. (B) Dose-dependent reduction of MCF-7 cells 24, 48 and 72 hrs after siRNA4 treatment compared to siRNA1 or siRNA4S treatment (5.6-566 nM). Growth inhibition of (C) SW-480, (D) HeLa S3 and (E) A549 cells over a period of 4 days. (F) Growth of normal human mammary epithelial cells (HMECs) over a period of 4 days after treatment with either siRNA1, siRNA4 or scrambled siRNA in different concentrations (56 nM-2 μM). All line graphs represent means of three independent experiments. Bar graphs represent means of 3 different experiments with 95% confidence intervals.

FIG. 7. Uptake of fluorescein-labelled siRNA4 by MCF-7 cells and HMECs. (A) FACScan analysis of control cells and fluorescein-labelled siRNA4-treated MCF-7 cells (left panel) and HMECs (right panel). (B) Based on the background fluorescence of control cells the uptake of fluorescein-labelled siRNA4 in MCF-7 cells and HMECs was determined.

FIG. 8. Reduction of PLK1 mRNA in vitro. (a) Relative positioning of the predicted hybridization sites of the 26 tested PLK1 ASOs. Sequences of all ASOs are available upon request. Arrows indicate positions of the potent ASOs P12 and P13. (b) Northern blot analysis of PLK1 mRNA in MDA-MB-435 cells 24 hrs after ASO treatment. HSV-ASO served as negative control. To ascertain uniformity of loading the membrane was reprobed with human,β-actin and G3PDH. Percent inhibition was calculated by comparison with standardized PLK1 mRNA levels of cells grown in the absence of ASOs. PLK1 mRNA expression in HeLa S3 cells (c) and A549 cells (d) 24 hrs after ASO application. Membranes were reprobed with human, β-actin.

FIG. 9. Inhibition of PLK1 protein expression and kinase activity by PLK1-specific, ASOs in cancer cells. Control cells were incubated with Opti-MEM. HSV-ASO served as negative control. Percent inhibition was calculated by comparison with PLK1 protein levels in cells grown in the absence of ASOs. (a) Western blot analysis of PLK1 protein in MDA-MB-435 cells 48 hrs after ASO treatment. Indicated are the signals for PLK1 and actin in both autoradiographs. Actin served as control for equal loading (upper panel). Quantification of PLK1 protein levels after normalization to actin was performed using a Kodak gel documentation system (ID 3.5). Mean values of at least 3 independent experiments with standard deviation are shown (lower panel). Quantification of PLK1 protein in HeLa S3 cells (b) and A549 cells (e) after normalization to actin. PLK1 protein correlated to the concentration of ASO P12 in HeLa S3 cells (d) and in MDA-MB-435 cells (e). PLK1 immunoprecipitated from ASO treated cells phosphorylated casein to a lower extent compared to PLK1 from control cells (upper panel). Coomassie staining served as control for equal loading of substrate (middle panel) Equal amounts of PLK1 were subjected to the enzymatic assay (lower panel) (f).

FIG. 10. Antiproliferative effects of PLK1 antisense inhibition in cancer cells. Control cells were incubated only with Opti-MEM. HSV-ASO served as a control. Percent inhibition was calculated by comparison to the number of cells treated with DOTAP grown in the absence of ASOs. (a) Growth inhibition of MDA-MB-435 cells treated with ASOs at a concentration of 250 nM over a period of 2 days. Growth inhibition of HeLa S3 (b) and A549 cells (c) over a period of 3 days. Dose-dependent reduction of MDA-MB-435 cells (d) and HeLa S3 cells (e) after ASO treatment. Mean values of at least 3 independent experiments with standard deviation are shown.

FIG. 11. ASO treatment induced abnormal centrosome distribution in A549 cells. Cells were transfected with PLK1 ASOs and immunostained 48 hrs post transfection for γ-tubulin. Interphase cells displayed abnormal localization of multiple centrosomes. Laserscan images (a and b) represent characteristic interphase figures.

FIG. 12. Effects of PLK1 ASOs on the growth of A549 tumors in nude mice. (a) Growth inhibition of two PLK1 ASOs (P12, P13) in contrast to control ASO (HSV)-treated and to untreated control mice. ASO administration was initiated 25 days after transplantation and continued for 24 days. Mean values of at least 3 independent experiments with standard deviation are shown (*: p<0.05). (b) Immunohistochernical analysis of PLK1 and MIB-I in A549 tumor xenografts after treatment. A, PLK1 expression in ASO (HSV)-treated animals. PLK1 expression in P12-treated animals. C, MIB-1 expression in ASO (HSV)-treated animals. D, MIB-1 expression in P12-treated animals.

FIG. 13. (A) Alignment of the polo-boxes from PLK1, Plk2 (Snk) and Plk3 (Fnk/Prk). Conserved amino acids are shown in bold. Mutations are underlined. (B) Wild-type polo-box P1 and mutated polo-box P2 internalized into cells efficiently. MCF-7 and HeLa S3 cells were treated with FITC-labeled P1 (a, e, c and g) or P2 (b, f, d and h) for 3 h and visualized using a CSLM. a-d: images of phase-contrast (40×). e-h: use of FITC-labeled peptides (40×). (C) Wild-type polo-box P1 exerts its inhibitory effect on proliferation in a dose dependent manner. MCF-7 cells were incubated with indicated concentrations of wild-type polo-box P1 on day 1 and 3. Cells were counted on day 3 and 7. Results were based on three independent experiments (mean and SEM).

FIG. 14. Wild-type polo-box exhibited an antiproliferative activity on MCF-7 (A), Saos-2 (B) and HeLa S3 cells (C). Cells were treated with 5 μM of indicated peptides on day 1, 3, 6 and 8, harvested and counted on day 3, 6, 8 and 10. Values represent mean +/− SEM of 3 independent experiments. (D) G₂/M population was increased 25-35% after treatment with the polo-box. FACS analysis of HeLa S3 cells on day 6: Cells were treated as described in (A-C) and analyzed with Cycle TEST™ PLUS DNA reagent kit (Becton Dickson). (E) The number of mitotic cells increased 3-4 fold after treatment compared to control cells. HeLa S3 cells were treated 24 h with different peptides at 5 μM and stained for DNA and α-tubulin for subsequent fluorescence analysis. To determine the percentage of the mitotic population 500 cells were inspected. Each experiment was repeated three times independently.

FIG. 15. Wild-type polo-box induced apoptosis in MCF-7 and HeLa S3 cells. MCF-7 (A) and HeLa S3 (B) cells were treated for 16 h, stained with annexin V and PI, and analyzed using a flow cytometer. Camptothecin treatment (10 μM) served as positive control. (C) Cells were treated for 1 day, stained with Hoechst 33342 and visualized with a fluorescence microscope (Leica). Upper panel: HeLa S3 cells treated with P1 and P3 (b-e), lower panel: MCF-7 (g-j). (D) Wild-type polo-box induced the misalignment of chromosomes and centrosomal abnormalities. Cells treated with peptides were further analyzed using DNA (a-c) or α-tubulin staining (d-f).

FIG. 16. Wild-type polo-box inhibited phosphorylation of substrates by PLK1 in vitro. PLK1 purified from Sf 9 cells was incubated with specific substrates at different concentrations of P1-P3 at 37° C. for 20 min. (A) Autoradiogram of phosphorylated substrates. (B) Input control (Coomassie staining) (C) Standardized phosphorylation using a gel documentation system (Kodak).

FIG. 17. Impact of expression of PLK1-specific hairpin RNAs driven by U6 promoter containing vectors on PLK1 expression in HeLa S3 cells in culture (A) Strategy for generating hairpin RNA from DNA templates (pBS/U6/shRNA/PLK1 and pBS/U6/shRNA/PLK1S). An inverted repeat is inserted at the +1 position of the U6 promoter (−315 to +1). The specific motif is 21-nt in length and corresponds to the coding region of PLK1. The two sequences that form the inverted repeat are separated by a spacer of 6-nt. A termination signal for transcription encompassing five thymidine residues is attached to the 3′ end of the inverted repeat. The transcribed RNA is predicted to fold back to form a small hairpin RNA (shRNA). The selection of the nucleotide sequence to be included in the shRNA vector is based on previous experiments using synthetic siRNA for efficient inhibition of PLKI in HeLa S3 cells (Spänkuch-Schmitt, 2002). (B) Impact of shRNA on PLK1 mRNA levels in the cervical cancer cell line HeLa S3. A Northern blot analysis was performed 72 hours after transfection with 3.0 μg, 4.5 μg or 6.0 μg of plasmid DNA (pBS/U6/shRNA/PLK1, pBS/U6/shRNA/PLK1S or pBS/U6) in each well of 6-well-plates. To control for variability of loading and transfer, membranes were reprobed for human β-actin, and actin-normalized PLK1 mRNA levels were compared. The amount of PLK1 mRNA expression remaining is given as a percentage of PLK1 mRNA levels in control cells that were incubated with only Opti-MEM I (neither shRNA-expressing plasmid nor FuGENE 6™). Bar graphs show means of 3 independent experiments and the upper 95% Cl.

FIG. 18. Ex vivo experiment to determine the influence of the nuclease inhibitor aurintricarboxylic acid (ATA) on the stability of plasmid DNA in murine blood. Blood was incubated at 37° C. with plasmids and ATA at indicated ratios for 5 min (A), 30 min (B), 2 hours (C), and 4 hours (D). Thereafter DNA was analyzed in a Southern blot analysis. As control linearized (Kpn I) and circular plasmids (L linear, C circle, S supercoiled) were also subjected to electrophoresis. Prolonged stability of supercoiled plasmid DNA is indicated by arrows (panel C, DNA:ATA=5:1 and DNA:ATA =0.5:1).

FIG. 19. Impact of expression of PLK1-specific hairpin RNAs driven by U6 promotorcontaining vectors on the growth of HeLa S3 cervical carcinoma cells and on PLK1 expression in a xenograft model using nude mice. (A) HeLa S3 tumors were transplanted subcutaneously into the flanks of nude mice. Plasmids and ATA (ATA:DNA=1:5) were administered to tumor-bearing mice by bolus intravenous injection 3 times a week (Monday, Wednesday and Friday) for 26 days. Tumor volume was determined using a caliper twice a week and calculated according to the formula V=π/6×largest diameter×smallest diameter². Displayed are the means of all tumor volumes for each group, and the upper 95% Cl. (B) Photographs of two mice following treatment: one mouse received treatment (26 days) with the parental plasmid pBS/U6 with ATA (lower panel), and a second mouse (upper panel) received treatment (26 days) with pBS/U6/shRNA/PLK1 with ATA. (C) Detection of plasmids in the tumors of mice from each of the four treatment groups. Primers against the parental vector pBS/U6 were chosen to detect all plasmids by PCR. PCR products are shown following separation by electrophoresis on a 1% agarose gel. (D) Effect of pBS/U6/shRNA/PLK1-mediated expression on PLK1 mRNA in tumors after 26 days of treatment. Tumors were excised one day after the last administration of plasmid, total mRNA was isolated and Northern blot analysis performed. To control for variability of loading gels were stained with ethidium bromide before blotting and PLK1 mRNA levels were normalized to the ethidium bromide staining of rRNAs and then compared. The amount of PLK1 mRNA expression remaining is given as a percentage of PLK1 mRNA levels in tumors treated with the parental vector pBS/U6. (E) Immunohistochemical analysis of PLK1 and Ki-67 levels in tumors with shRNA/PLK1 or pBS/U6 treatment. PLK1 staining of tumors excised from mice after pBS/U6/shRNA/PLK1 treatment [a] or pBS/U6 treatment [b], and Ki-67 staining of tumors excised from mice after pBS/U6/shRNA/PLKI treatment [c] or pBS/U6 treatment [d] are shown.

FIG. 20 shows a tumour growth curve of experiments with application of the same plasmids as in FIG. 19 to xenograft nude mice bearing A549 lung tumours.

### EXAMPLE 1

Inhibition of PLK1 Activity by siRNAs

1.1 siRNAs and Antibodies

siRNAs were from Dharmacon Research Inc. (Colorado, USA). siRNA sequences targeting PLK1 (NCBI accession number: X75932) correspond to positions 178-200 (siRNA2), 362-384 (siRNA3), 1416-1438 (siRNA4) and 1572-1594 (siRNA5) located within its open reading frame. siRNA1 directed against lamin A/C (NCBI accession number: X03444) represents positions 608-630 relative to the start codon (Elbashir et al., 2001). siRNA4S (scrambled) representing siRNA4 as random sequence was used as control. Monoclonal PLK1 antibodies were obtained from Transduction Laboratories (Heidelberg) for Western blots and from Zytomed (San Francisco, USA) for kinase assays. Monoclonal antibodies for lamin were purchased from Santa Cruz Biotechnologies (Heidelberg) and for actin from Sigma (Deisenhofen).

1.2 Cell Culture

Ham's F12 and FCS were purchased from PAA Laboratories (Cöbe). DMEM, RPMI 1640, PBS, Opti-MEM, oligofectamine, glutamine, penicillin/streptomycin and trypsin were from Invitrogen (Karlsruhe). Tumor cell lines SW-480 (colon), MCF-7 (breast) and HeLa S3 (cervix) were provided by DSMZ (Braunschweig) and A549 (lung) by CLS (Heidelberg). Mammary epithelial basal medium (MEBM), growth medium supplements (MEGM SingleQuots) and the human mammary epithelial cell system (HMEC) were obtained from Clonetics (Verviers, Belgium). For the isolation of HMECs human breast tissue was removed from patients, passaged several times to select desired epithelial cells and cryopreserved in sixth or seventh passage. For experimental use cryopreserved cultures were assured for fifteen population doublings by the manufacturer. Cell culture was performed according to the supplier s instructions.

1.3 In Vitro Administration of siRNAs

Four siRNAs, each 21 nt in length (siRNAs2-5) with symmetric 3′ overhangs of 2 deoxythymidines, directed against the open reading frame of PLK1 were tested for their potential of PLK1-specific interference in tumor cells and in primary human mammary epithelial cells (HMEC). To evaluate the specificity of the siRNA action against PLK1, a scrambled siRNA (siRNA4S) and a siRNA targeted against lamin (siRNA1) were used as internal controls. All siRNAs (siRNAsl-5, siRNA4S) were tested for their potential to alter the expression of different genes (PLK1, lamin, actin).

Cancer cells and HMECs were transfected with siRNAs using the oligofectamine protocol according to the manufacturer s instructions:

In brief, 1 day prior to transfection cancer cells were seeded without antibiotics to 5×10⁵ cells/25 cm²-culture flask corresponding to a density of 40-50% at the time of transfection. In all experiments cells were treated with siRNAs2 5 (PLK1), siRNA1 (lamin) or siRNA4S (scrambled) at a concentration of 56 nM. Cells incubated with Opti-MEM alone without siRNA were considered as control cells. Incubation of cells with siRNAs in Opti-MEM with oligofectamine or with Opti-MEM alone at 37° C. for 4 hrs was followed by addition of fresh culture medium with threefold FCS. All transfections were performed in triplicate for each time point, e.g. for analysis of PLK1 mRNA after 6, 24 and 48 hrs and for analysis of PLK1 protein after 48 hrs. Subsequently, the inhibitory activity of siRNAs2-5 against PLK1 was standardized to the effect exerted by siRNA4S (scrambled). Furthermore, after treatment with siRNA1 the knockdown of lamin was evaluated in MCF-7 cells and HMECs to assess RNAi against a non-tumor associated gene. The growth rate of 5×10⁵ cells was determined over a period of 4 days by counting cells after 24, 48, 72 and 96 hrs in triplicate.

In the dose-dependence experiments using siRNA1, siRNA4 and siRNA4S (scrambled) at concentrations between 0.5 nM and 566 nM we focused our attention predominantly on the potency of siRNA4, because previous tests had revealed the pronounced inhibitory potential of siRNA4 in different cell lines.

Transfection of HMECs was carried out as described above for cancer cell lines. In dose-dependence experiments with HMECs concentrations ranged from 566 nM to 2 μM because initial studies demonstrated that concentrations sufficient to inhibit proliferation of cancer cells had no substantial effect on HMECs. For the determination of PLK1 mRNA, lamin protein, immunofluorescence and FACScan analysis the concentration of siRNAs was 2 μM. Normal culture medium with threefold growth supplements (SingleQuots) was added after 4 hrs incubation at 37° C. with siRNAs or with Opti-MEM alone. Additional manipulations of HMECs correspond to the techniques used for cancer cell lines.

1.4 RNA Preparation and Northern Blots

For the isolation of total RNA an RNeasy mini-kit was used according to the manufacturer s protocol (Qiagen, Hilden). Radiolabeling of antisense strands for PLK1 and β-actin was performed using 100-250 μCi of [α-³²P]dCTP (6000 Ci/mmol) for each reaction, 50 μM of each other dNTP and 10 pmol (each) of primer PLK1-17-low (5′ -tgatgttggcacctgccttcagc-3′) corresponding to position 1533-1554 within the open reading frame of PLK1 or actin-2-low (5′-catgaggtagtcagtcaggtc-3′) as described previously (Wolf et al., 1997). Probes corresponding to aa 285-497 of PLK1 were generated by PCR. Northern blotting and hybridizations were carried out as reported (Wolf et al., 1997).

6, 24 and 48 hrs after siRNA treatment of cell lines (MCF-7, HeLa S3, SW-480 and A549) and 48 hrs after siRNA treatment of HMECs total RNAs were subjected to Northern blot analyses to monitor the impact of siRNA with time. In order to compare PLK1 expression among different treatments and time points, it was normalized to actin signals (control for equal loading). In addition, resulting normalized PLK1 mRNA levels were displayed in relation to siRNA4S-treated cells to differentiate effects of PLK1 inhibition from transfection- or random siRNA-related effects.

1.5 Western Blot Analysis

48 hrs after siRNA treatment cancer cells (MCF-7, HeLa S3, A549, SW-480) were lysed for Western blotting as described (Hock et al., 1998). For this purpose 50 μg of total protein were separated on a 12% SDS-polyacrylamide gel. Membranes were kept for 1 hr with monoclonal antibodies for PLK1 (1:250) and actin (1:200.000) or with monoclonal antibodies against lamin (1:100) and actin (1:200.000) followed by incubation with goat anti-mouse serum (1:2.000) for 30 min.

For lysis of HMECs 48 hrs after siRNA treatment cells were rinsed with PBS, removed from culture flasks, spun down and directly lysed in SDS buffer (4% SDS, 20% glycerol, 0.12 M TRIS, pH 6.8) containing a protease inhibitor cocktail (Boehringer, Mannheim). Lysates were immediately boiled for 10 min and protein concentration was measured (Garbe et al., 1999). 100 μg of total protein from HMECs were separated on a 12% SDS-polyacrylamide gel. Membranes were kept for 1 hr with monoclonal antibodies for PLK1 (1:50) and actin (1:200.000) or with monoclonal antibodies against lamin (1:100) and actin (1:200.000), respectively. Western blots were performed as described (Kauselmann et al., 1999).

In order to compare protein levels in different experiments, PLK1 protein expression was routinely normalized to actin levels as described above for PLK1 mRNA. In addition, resulting normalized PLK1 protein levels were displayed in relation to siRNA4S-treated cells to differentiate effects of PLK1 inhibition from transfection- or random siRNA-related effects.

1.6 Kinase Assays

To determine whether downregulation of PLK1 mRNA and protein by siRNA was accompanied by a reduction of endogenous kinase activity, 48 hrs after siRNA treatment cells were lysed, endogenous PLK1 was immunoprecipitated using monoclonal PLK1 antibodies (0.5 μg antibody for each kinase reaction were added to 800 μg of total protein), incubated with 0.5 -1 μg of substrate and 2 μCi of [γ-³²P]ATP for 30 min at 37° C. in kinase buffer (20 mM HEPES pH 7.4, 150 mM KCl, 10 mM MgCl₂, 1 mM EGTA, 0.5 mM DTT, 5 mM NaF, 0.1 mM Na₃VO₄). The cytoplasmic retention signal (CRS, aa 100-159) within human cyclin B1 was used as substrate. Products from kinase reactions were fractionated on 12% SDS-polyacrylamide gels (BioRad, München) and subjected to autoradiography.

1.7 Determination of Cell Proliferation

After siRNA treatment cell numbers were determined (24, 48, 72 and 96 hrs) using a hemacytometer. Cell viability was assessed by trypan blue staining. The number of control cells (incubated with Opti-MEM without siRNA) after 96 hrs was used as reference for this analysis. The ratio of siRNA-treated cells and control cells was determined to gain the percentage of proliferating cells. Each experiment was performed in triplicate and the standard deviation for each group was determined.

1.8 Indirect Immunofluorescence

Antibodies directed against α-tubulin to visualize the spindle apparatus and α-tubulin to localize centrosomes were used. Furthermore, polyclonal PLK1 -antibodies were used to determine intracellular levels of PLK1. DNA staining was performed using DAPI, Hoechst 33342; BisBenzimide (Sigma, Deisenhofen). Antibodies were used as follows: polyclonal rat α-tubulin (Serotec/Biozol, Eching) 1:100, monoclonal mouse α-tubulin (Dianova, Hamburg) 1:100, polyclonal rabbit PLK1 (Yuan et al., 1997) 1:100 or monoclonal γ-tubulin (Sigma) 1:100. 48 hrs after siRNA treatment cells were stained as described before (Kauselmann et al., 1999). Cells were analyzed at a magnification of 40× with a fluorescence microscope (Leica) or a confocal laser scan microscope (CLSM, Zeiss) using a 100× oil immersion objective.

1.9 FACScan Analysis

48 hrs after transfection with siRNAs an analysis of cell cycle distribution and apoptosis was carried out using a Becton Dickinson FACScan. Cells were harvested, washed with PBS and probed with CycleTEST™ PLUS DNA reagent kit (Becton Dickinson) according to the manufacturer's protocol to determine the cell cycle distribution of siRNA-treated cells. For each treatment (control, siRNAs2-5 (PLK1), siRNA1 (lamin) and scrambled siRNA4S) 30.000 cells were analyzed in triplicate. Cell cycle distribution in percentage was calculated using ModFit LT for Mac. The percentage of cells in G2/M phase was compared to control, siRNA1- or siRNA4S-treated cells. For the detection of apoptotic phenotypes cells were harvested 48 hrs after transfection, fixed with ice-cold 70% ethanol, treated with RNase A (5 μg/ml) and stained with propidium iodide (50 μg/ml). Subsequent analyses were performed using the CELLQuest software.

To find out whether effects exerted by siRNAs in cell culture are influenced by different transfection efficiencies, we determined the uptake of fluorescein-labelled siRNA4 into MCF-7 cells and HMECs 24 hrs after transfection by FACScan analysis. Considering the background fluorescence of control cells (incubated with Opti-MEM, no siRNA treatment), the fluorescence of 10.000 cells was determined. This analysis was performed using the CELLQuest software.

1.10 Quantitative Analysis

Quantification of PLK1 and actin expression was performed using a Kodak gel documentation system (1D 3.5). To determine Northern and Western blot signal intensities, autoradiographs were scanned. Integration of signal intensities was followed by quantitative comparison of PLK1 and actin expression, i.e. for each treatment the ratio of PLK1 and actin signals was determined. Values were given in percent compared to siRNA4S-treated cells.

1.11 Statistical Methods

Each Western blot experiment was performed three or four times. Northern blots were performed in triplicate. Signal intensities were normalized to actin and means calculated thereafter. For the determination of proliferation cell numbers were determined in triplicate at each timepoint. FACScan analyses were carried out three times for each cell type. Statistical analysis was performed with two-way ANOVA to consider random effects of individual gels and different siRNA treatments. For this purpose all siRNA treatment groups were compared to siRNA4S-treated cells. p-values and 95% confidence intervals (Cl) for each siRNA treatment, which induced significant changes of the analyzed parameters are given. If the lower 95% confidence interval was below zero it was given as zero, because in biological systems negative values are inappropriate.

1.12 Specific Inhibition of PLK1 mRNA and Protein Expression by siRNAs

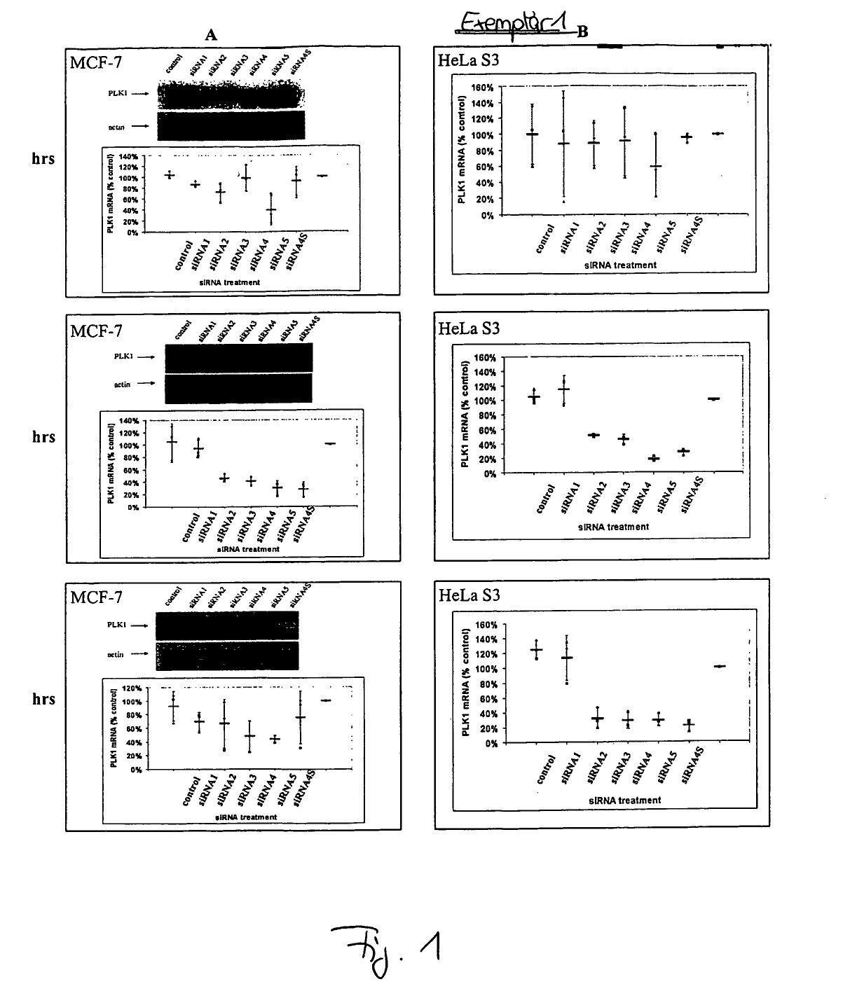

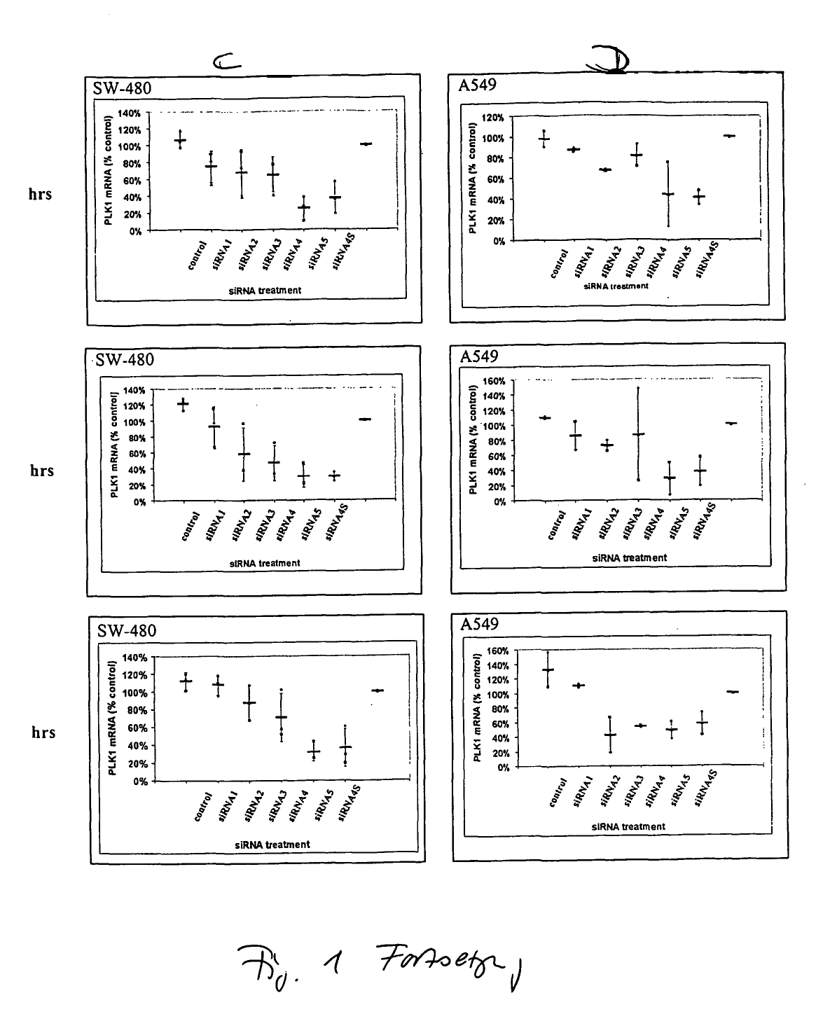

Because siRNA acts by decreasing the half-life of RNA, the natural stability of RNA will have a quantitative influence upon its suitability as target for gene silencing (Elbashir et al., 2001). Thus, we started our study by testing siRNAs for their ability to reduce the endogenous level of PLK1 mRNA in different cancer cell lines (MCF-7, HeLa S3, SW-480 and A549). Northern blot analyses standardized to the expression of actin were performed. Treatment of MCF-7 breast cancer cells in vitro with siRNAs2-5 at a concentration of 56 nM in the presence of oligofectamine did not reduce PLK1 mRNA significantly 6 hrs after transfection but led to a significant loss of PLK1 mRNA within 24 hrs and 48 hrs (FIG. 1A). siRNA4S representing siRNA4 as scrambled version was used to differentiate effects of PLK1 inhibition from transfection- or random siRNA-related events. The study revealed that all tested siRNAs targeted against PLK1 reduced PLK1 mRNA in human MCF-7 cells to levels of 20-40% compared to cells treated with siRNA4S (after 24 hrs: siRNAs2-5: p<0.01; siRNA2: 54%, 95% Cl=29-62; siRNA3: 59%, 95% Cl=22-60; siRNA4: 70%, 95% Cl=2-58; siRNA5: 72%, 95% Cl=0-57; after 48 hrs: siRNA4: 56%, p<0.01, 95% Cl=31-56). Suppression by siRNAs2-5 could not only be demonstrated for MCF-7 cells but also for other tumor cell types like HeLa S3, SW-480 and A549 (FIG. 1B-D). After 24 hrs siRNAs2-5 reduced PLK1 mRNA in HeLa S3 cells significantly (siRNA2: 49%, p<0.001, 95% Cl=46-57; siRNA3: 54%, p<0.01, 95% Cl=29-62; siRNA4: 82%, p<0.001, 95% Cl=9-27; siRNA5: 72%, p<0.01, 95% Cl=17-39). After 48 hrs inhibition did not differ substantially (siRNA3: 71%, p<0.01, 95% Cl=3-55; siRNA4: 71%, p<0.01, 95% Cl=9-49; siRNA5: 78%, p<0.01, 95% Cl=5-39). In SW-480 cells the reduction of PLK1 mRNA occurred rather rapidly within 6 hrs, but it was significant only for siRNA4 and siRNA5 at all three timepoints (after 6 hrs: siRNA4: 75%, p<0.05, 95% Cl=0-61; siRNA5: 63%, p<0.05, 95% Cl=0-84; after 24 hrs: siRNA4: 70%, p<0.05, 95% Cl=0-65; siRNA5: 70%, p<0.01, 95% Cl=17-42; after 48 hrs: siRNA4: 68%, p<0.01, 95% Cl=5-58; siRNA5: 63%, p<0.05, 95% Cl=0-89). In A549 cells a significant reduction of PLK1 mRNA was found for the following siRNAs: after 6 hrs: siRNA2: 33%, p<0.001, 95% Cl=45-69; siRNA5: 59%, p<0.01, 95% Cl=25-57; after 24 hrs: siRNA2: 27%, p<0.05, 95% Cl=56-90; siRNA4: 71%, p<0.05, 95% Cl=0-82; siRNA5: 62%, p<0.05, 95% Cl=0-85; after 48 hrs: siRNA3: 46%, p<0.001, 95% Cl=52-56; siRNA4: 51%, p<0.05, 95% Cl=21-78; siRNA5: 42%, p<0.05, 95% Cl=19-96.

In addition to cancer cells we were interested to test siRNA-mediated effects in primary cells. Human mammary epithelial cells (HMECs) were evaluated for their sensitivity towards siRNAs. Concentrations of siRNAs (5.6-566 nM) suitable for reduction of mRNA levels in cancer cells did not cause downregulation in HMECs. A significant reduction of PLK1 mRNA at 2 μM was detected 48 hrs after siRNA treatment for siRNA3 (54%, p<0.05, 95% Cl=12-79) and siRNA4 (63%, p<0.05, 95% Cl=0-91) compared to siRNA4S, but not for siRNA2 and siRNA5 (FIG. 1E).

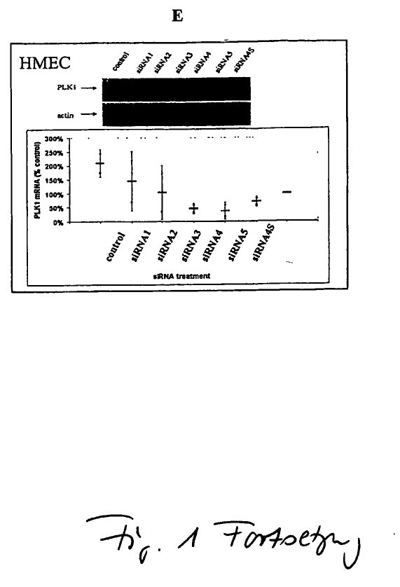

Subsequently, we determined whether decreases in PLK1 mRNA were accompanied by a reduction of PLK1 protein. The expression of PLK1 protein in different tumor cell lines was evaluated 48 hrs after single application of siRNAs. Significant inhibition of the 68 kDa-PLK1 protein in MCF-7 cells compared to siRNA4S-treated cells was observed with siRNAs2-5 (siRNA2: 89%, p<0.01, 95% Cl=0-33; siRNA3: 85%, p<0.01, 95% Cl=0-31; siRNA4: 95%, p<0.01, 95% Cl=0-22 and siRNA5: 91%, p<0.01, 95% Cl=0-33), which were previously shown to reduce PLK1 transcript levels significantly (FIGS. 2A). A reduction of PLK1 mRNA induced by the application of siRNAs2-5 led also to a significantly lower level of the corresponding protein in HeLa S3 (siRNA2: 70%, p<0.05, 95% Cl=0-71; siRNA3: 79%, p<0.01, 95% Cl=7-35; siRNA4: 65%, p<0.05, 95% Cl=0-88; siRNA5: 73%, p<0.01, 95% Cl=7-55), SW-480 (siRNA2: 59%, p<0.05, 95% Cl=0-90; siRNA3: 63%, p<0.01, 95% Cl=21-54; siRNA4: 80%, p<0.01, 95% Cl=0-44; siRNA5: 91%, p<0.01, 95% Cl=0-36) and A549 cells (siRNA3: 66%, p<0.05, 95% Cl=0-86; siRNA4: 83%, p<0.01, 95% Cl=0-38; siRNA5: 62%, p<0.05, 95% Cl=0-82) (FIG. 2B-D).

PLK1 mRNA and protein levels were not influenced significantly by the scrambled siRNA4S or by siRNA1 targeted to lamin proposing that the above described effects on cells treated with siRNAs are sequence-specific for PLK1 (FIG. 1A-D, 2A-D). This notion gained further support from the observation that siRNAs2-5 did not alter the expression of actin mRNA and protein (FIG. 1A, 2A). Furthermore, varying concentrations of siRNA4, which had the most pronounced inhibitory effect in all cell lines tested, led to the reduction of PLK1 protein (FIG. 2E). While the treatment of MCF-7 cells with siRNA4 at a concentration of 0.5 nM did not alter the level of PLK1 protein substantially, concentrations between 5.6 and 56 nM exhibited a pronounced inhibitory effect (71% and 89%, respectively). Elevating the concentration to 566 nM diminished the inhibitory potential of siRNA4, which might be due to a reduced transfection efficiency at high siRNA concentrations. PLK1 protein expression was independent of different concentrations of siRNA1 and siRNA4S.

Furthermore, we examined the concerted uptake of several siRNAs into MCF-7 cells and co-transfected siRNA4 in combination with siRNA2, siRNA3 or siRNA5. PLK1 protein expression was completely downregulated in co-transfected cells (data not shown).

Immunoprecipitated PLK1 from MCF-7 cells treated with siRNA4 or with siRNA4S for 48 hrs was subjected to kinase assays to determine endogenous PLK1 kinase activity. In siRNA4-treated cells phosphorylation of the cytoplasmic retention signal of cyclin B1 as exogenous substrate (Toyoshima-Morimoto et al., 2001) was reduced to 18% of the level in control cells (FIG. 2F). In contrast, siRNA4S did not reduce kinase activity of endogenous PLK1 substantially. Taken together, PLK1 expression can be specifically reduced by siRNA treatment of cancer cells associated with downregulation of PLK1 activity.

In addition to the evaluation of transcript levels in HMECs we intended to test the impact of siRNAs on protein expression. However, the level of PLK1 in untreated HMECs was below the limit of detection. This result is in line with our previous observations showing that the level of PLK1 protein is very low in primary cells with weak proliferative activity (Yuan et al. 1997).

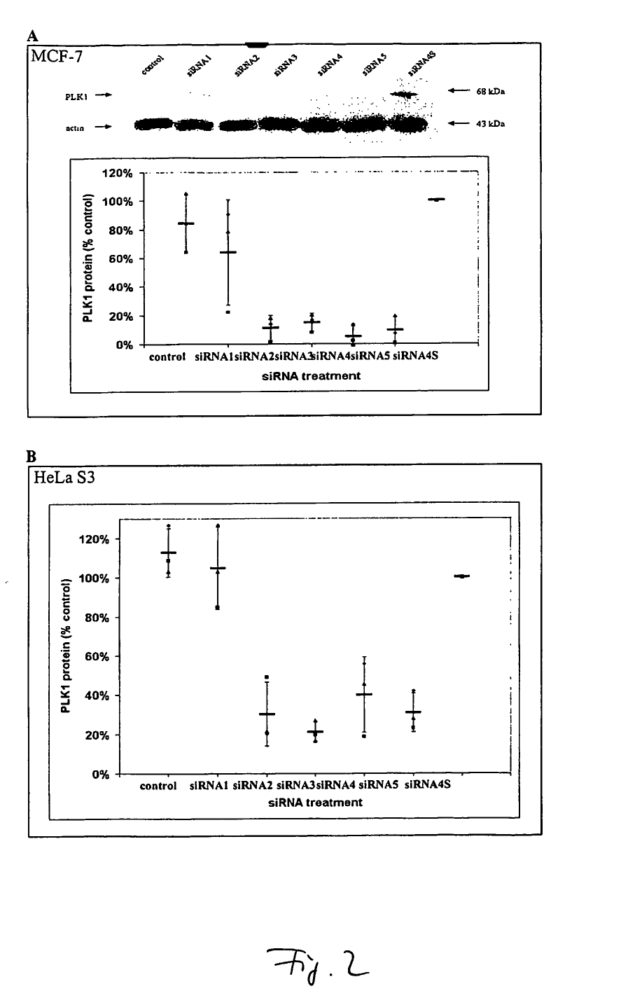

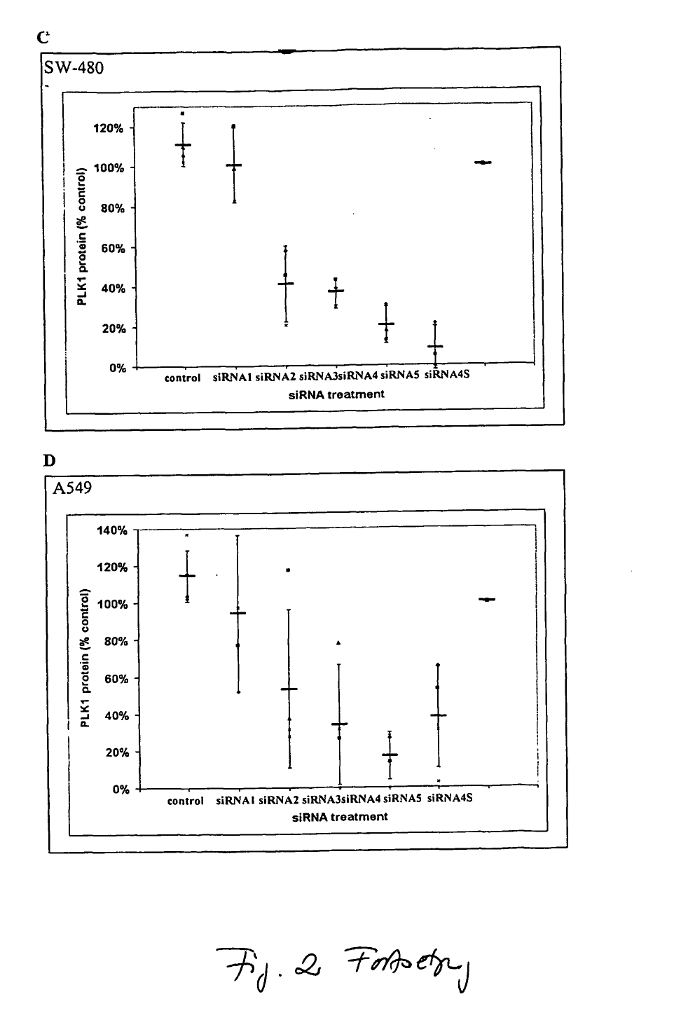

Moreover, we wondered whether the inhibition of a non-tumor gene like lamin is comparable to the differential downregulation of PLK1 in MCF-7 cells and HMECs. The analysis revealed that in both cell types lamin protein disappeared completely (FIG. 3A and B). However, the required concentrations differed markedly: For maximal reduction of lamin protein 56 nM siRNA1 was required in MCF-7 cells, in HMECs 2 μM was necessary. Since the downregulation of both proteins (PLK1, lamin) required high concentrations of siRNA in HMECs, this effect did not seem to be gene-specific. It is rather likely that primary cells require elevated levels of siRNA for efficient knock-down of gene expression.

1.13 Abrogation of Spindle Formation Induced by Reduced Levels of PLK1 Protein

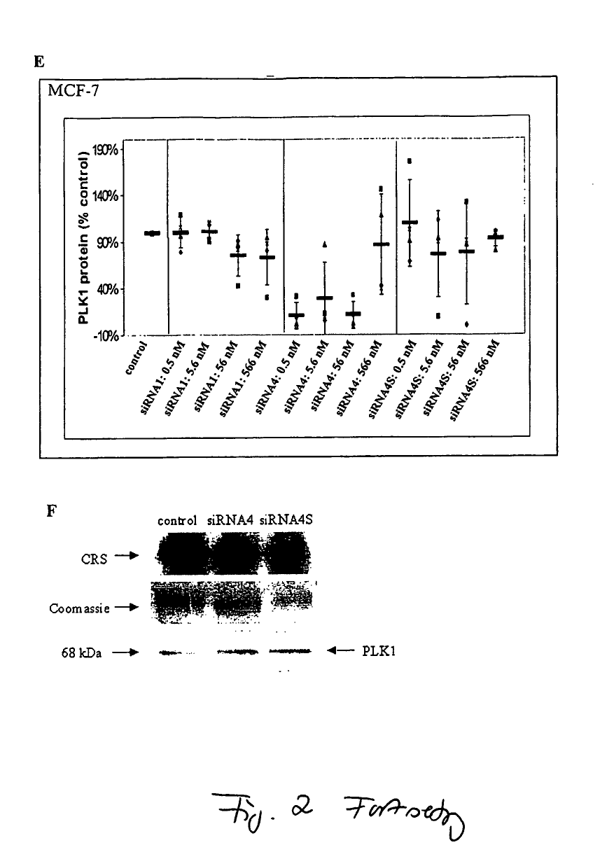

In previous studies microinjection of PLK1-specific antibodies induced abnormal distribution of condensed chromatin and monoastral microtubule arrays that were nucleated from duplicated but unseparated chromosomes (Lane and Nigg, 1996). Here we focused on analyzing the phenotype of siRNA-treated cells that exhibit a major downregulation of PLK1 expression in cancer cells. For the following studies siRNA4 was predominantly used for the reduction of PLK1 expression because different experiments had revealed that siRNA4 is a powerful inhibitor in different cancer cell lines. While control cells proceeded through mitosis, cells incubated with siRNA4 arrested in different mitotic stages depending on the cell type. SW-480 cells treated with siRNA4 did not enter prophase as can be derived from the lack of prophase typical chromosome condensation in the nuclei of DAPI-stained cells (FIG. 4A). However, many cells were found with separated centrosomes which moved to opposite ends of the nucleus (FIG. 4A, upper panel: γ-tubulin). Centrosomes were devoid of any microtubule connection (FIG. 4A, upper panel: α-tubulin). According to their DAPI-fluorescence nuclei contained 4N DNA although no chromatin condensation was detected. Thus, while the centrosomes performed prophase-typical separation, nuclei seem to persist in G₂-phase. In control cells centrioles organized astral microtubules in early prophase and displayed chromosome condensation in the nucleus (FIG. 4A, lower panel).

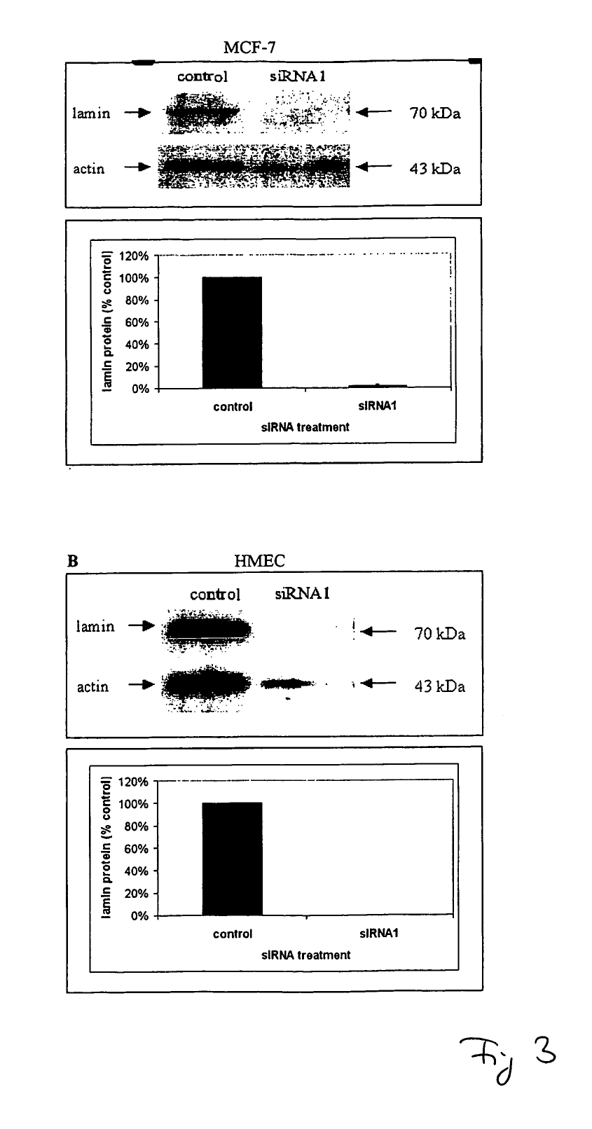

MCF-7 cells on the other hand displayed numerous apoptotic nuclei in DAPI-stained cultures but no mitotic stages after siRNA4 treatment (FIG. 5B). Examination of the supernatant of these cultures revealed a high percentage of mitotic cells, which obviously lost substrate adhesion. Metaphase or telophase chromosomal arrangements were rarely identified. More than 90% of all mitotic stages were characterized by highly condensed, knob-like chromosomes which remained in an overall structure resembling the shape of a nucleus (FIG. 4B). Only a few chromosomes left this ensemble lying in the immediate vicinity. This phenotype indicates that the nuclear envelope has disappeared but no further, mitotic spindle-related arrangement of chromosomes occurred. Chromatid separation did not take place. γ-tubulin was distributed all over the cytoplasm of these cells which also did not contain any microtubules (data not shown). The lack of microtubules could be a consequence of cell death after loss of adhesion.

Since a concentration of 56 nM, which induced severe phenotypical changes in cancer cells, had no effect in HMECs, we increased the concentration of siRNA4 to 2 μM. Still, morphological alterations in HMECs could not be detected (FIG. 4C).

Immunofluorescence staining of siRNA4-treated MCF-7 cells and HMECs revealed a marked reduction of PLK1 protein in both cell types. While MCF-7 control cells exhibited numerous normal mitotic figures, frequent impaired mitoses were found in siRNA4-treated cells, which are devoid of PLK1 protein (FIG. 4D, lower panel). Since mitotic HMECs are very hard to detect, FIG. 4D depicts cells in interphase. PLK1 expression in siRNA4-treated HMECs was at the level of detection (FIG. 4D, upper panel).

1.14 G₂/M Cell Cycle Arrest and Apoptosis in Cancer Cells In Vitro Induced by siRNA Treatment Targeted to PLK1

FACScan analyses revealed a strong G₂/M arrest in cancer cells induced by siRNA4: SW-480 (fivefold), MCF-7 (threefold), HeLa S3 (fivefold) and A549 cells (twofold). In contrast to the tested cancer cells, HMECs just exhibited weak G₂/M arrest (increase of 32%) following treatment with 2 μM siRNA4 (FIG. 5A, left panel). The effect of siRNA5 on cell cycle distribution was similar compared to siRNA4. While in MCF-7 and SW-480 cells siRNAs2 and 3 induced only weak G₂/M arrest (increase of 10-40%) compared to control cells, in HeLa S3 and A549 cells the G₂/M arrest exerted by siRNAs2 and 3 was comparable to siRNA4. siRNA1- or siRNA4S-treated cells exhibited no substantial change in cell cycle distribution compared to control cells (FIG. 5A, right panel). Staining of DNA in MCF-7 cells versus HMECs revealed an elevated number of apoptotic nuclei in siRNA4-treated MCF-7 cells compared to control cells (FIG. 5B, lower panel). In contrast, in siRNA4-treated HMECs no apoptotic phenotype was detectable (FIG. 5B, upper panel). In addition, CLSM experiments revealed an elevated number of apoptotic cells with disintegrated nuclear membranes and condensed chromatin in MCF-7 cells (FIG. 5C). To address this observation in more detail, we asked the question whether gene silencing of PLK1 causes apoptosis in different tumor types. The increase of Sub2N DNA content was determined by FACScan analysis. While control cells exhibited 1-5% Sub2N DNA, PLK1-specific siRNA4 induced increased Sub2N DNA in SW-480 (17%), MCF-7 (33%), HeLa S3 (50%) and A549 cells (13%) 48 hrs after transfection. In contrast, in HMECs siRNA4 treatment did not increase the ratio of Sub 2N DNA suggesting a differential effect of siRNA in cancer cells compared to primary epithelial cells.

1.15 Growth of Cancer Cells in Vitro Inhibited by PLK1-Specific siRNAs

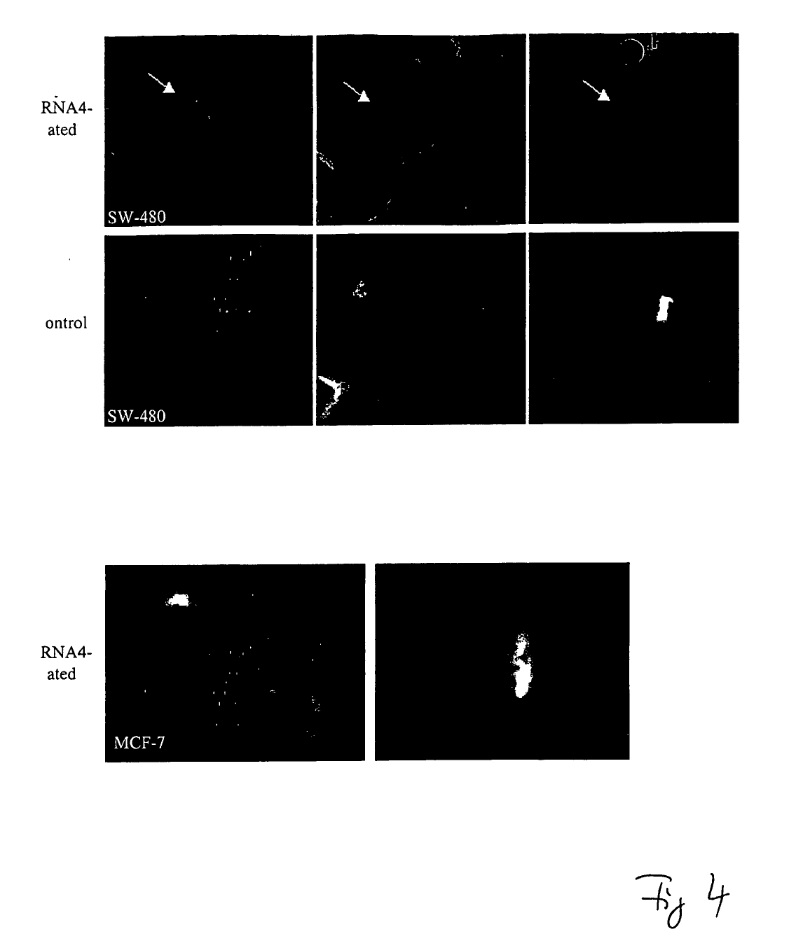

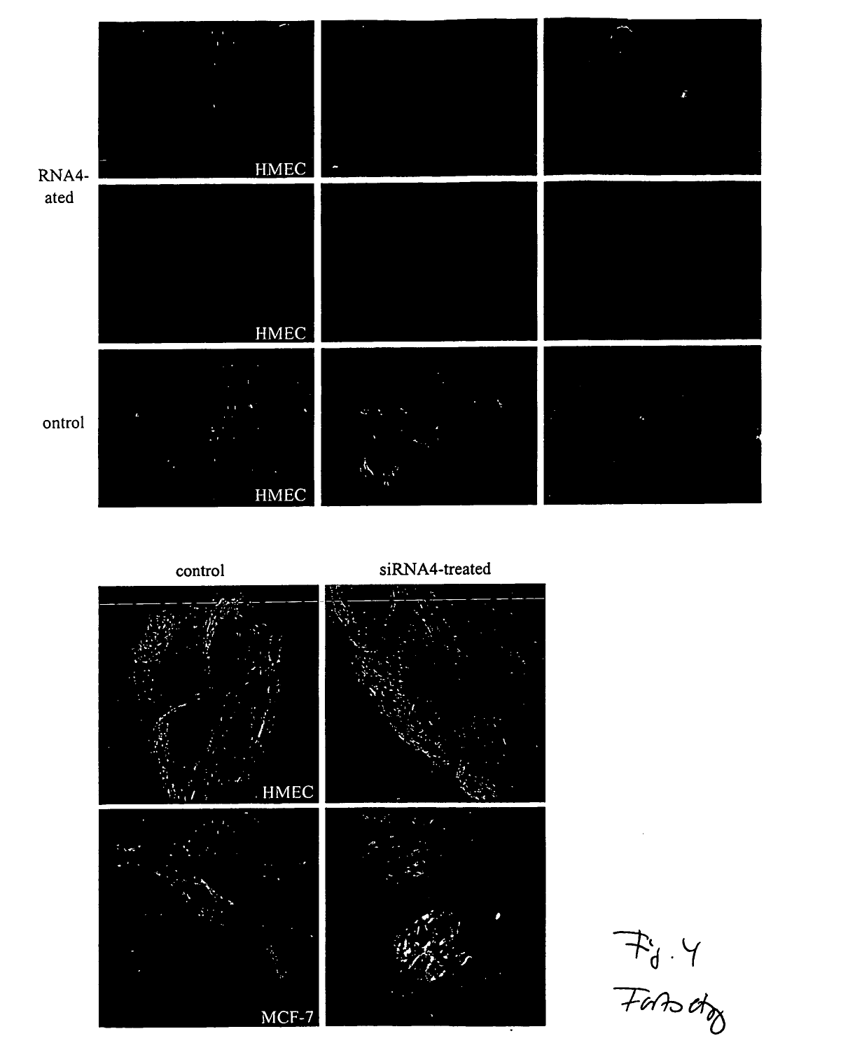

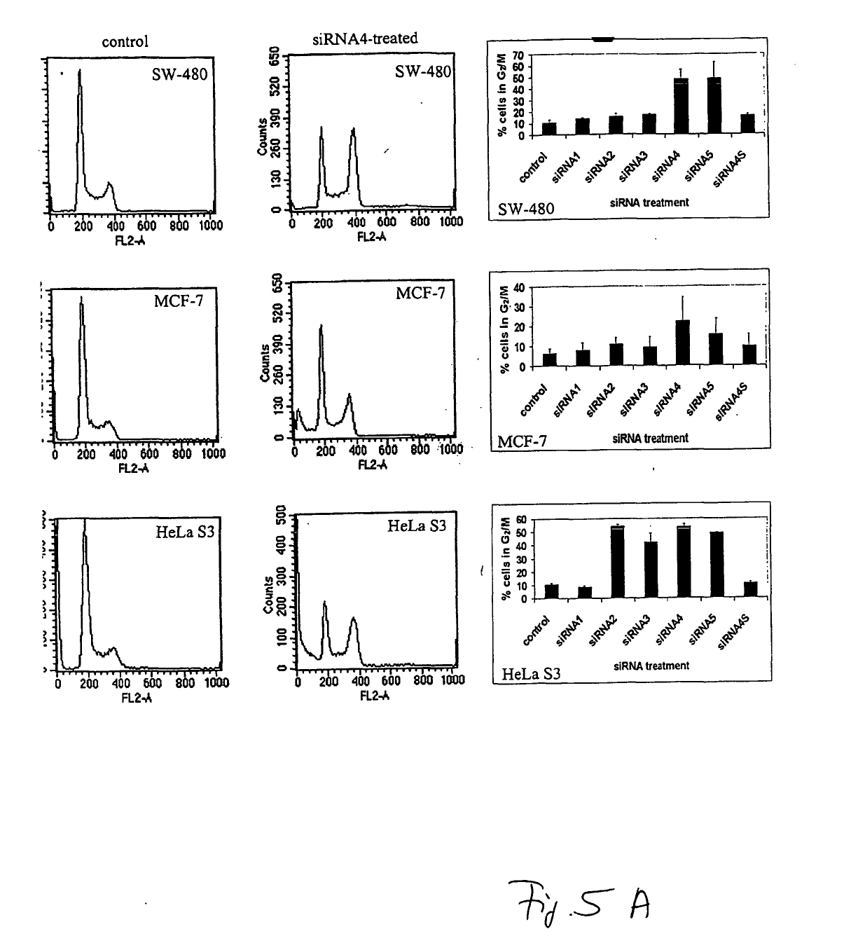

MCF-7 cells were treated with 56 nM siRNAs1-5 to determine whether siRNAs influence the proliferation of tumor cells. While the treatment with the transfection agent (oligofectamine) alone did not affect the growth rate of MCF-7 cells, treatment with siRNA1 (lamin) exhibited limited impact (FIG. 6A). In contrast, siRNAs2-5 had a significant antiproliferative effect compared to untreated MCF-7 control cells within 96 hrs (siRNA2: 83%, p<0.05, 95% Cl=0-73; siRNA3: 81%, p<0.05, 95% Cl=0-51; siRNA4: 97%, p<0.01, 95% Cl=0-18; siRNA5: 89%, p<0.05, 95% Cl=0-32) (FIG. 6A). Specificity of the siRNA-mediated inhibition was also indicated by a dose-dependent reduction of cell growth (FIG. 6B). Treating MCF-7 cells with siRNA1 (lamin) or with the scrambled siRNA4S demonstrated that both siRNAs did not reduce cell numbers substantially at any concentration tested compared to oligofectamine-treated cells. In contrast, increasing concentrations of siRNA4 (5.6-566 nM) led after 48 hrs to almost complete cell death (FIG. 6B). Subsequently, it was examined whether the reduction of proliferative activity is cell type-specific or can also be achieved in other cancer cell lines. siRNAs2-5 were shown to inhibit cell growth also significantly in SW-480 (siRNA2: 67%, p<0.01, 95% Cl=21-45; siRNA3: 75%, p<0.01, 95% Cl=23-27; siRNA4: 97%, p<0.001, 95% Cl=1-5; siRNA5: 97%, p<0.01, 95% Cl=1-5), HeLa S3 (siRNA2: 94%, p<0.05, 95% Cl=6-6; siRNA3: 91%, p<0.05, 95% Cl=2-16; siRNA4: 99%, p<0.05, 95% Cl=0-2; siRNA5: 98%, p<0.05, 95% Cl=0-9) and in A549 cells (siRNA2: 71%, p<0.01, 95% Cl=21-38; siRNA3: 66%, p<0.01, 95% Cl=32-36: siRNA4: 75%, p<0.01, 95% Cl=23-27; siRNA5: 73%, p<0.01, 95% Cl=25-29) suggesting that PLK1 expression is essential for the proliferation of different types of cancer cells (FIG. 6C-E).

Subsequently, the growth behaviour of the tumor cell line MCF-7 was compared to HMECs. To gain the same extent of growth reduction in HMECs, a 350fold higher concentration of siRNA4 was required compared to MCF-7 cancer cells. While in MCF-7 cells a growth reduction of 84% could be achieved 24 hrs after transfection with siRNA4 concentration of 5.6 nM, in HMECs 2 μM were required for a growth reduction of 78% (FIG. 6F). This observation suggested an elevated sensitivity of cancer cells towards siRNA directed against PLK1 compared to primary epithelial cells.

1.16 Differential Uptake of siRNAs in MCF-7 Cells Compared to Primary Human Mammary Epithelial Cells

Different lines of evidence (Northern and Western blots, CLSM- and FACScan-analyses) indicated that concentrations of PLK1 -specific siRNA4 between 5.6 and 566 nM, which are sufficient to inhibit PLK1 expression and growth of different cancer cells efficiently, had no obvious effect on HMECs. Thus, we asked the question whether different transfection efficiencies could be the reason for these observations. To compare effects of siRNA treatment between cancer cell lines and HMECs, the uptake of fluorescein-labelled siRNA4 was measured. A FACScan analysis of 10.000 cells revealed that at a concentration of 56 nM 89.8% MCF-7 cells were transfected but only 49.2% of HMECs. In HMECs a concentration of 2 μM was necessary to transfect 75.7% of the cells. These differences could be due to a different permeability of cell membranes in tumor cells versus primary cells (Mullin et al. 2000).

### EXAMPLE 2

Inhibition of PLK1 Activity by Antisense Oligonucleotides

2.1 ASOs Inhibit Specifically the Expression of PLK1 mRNA and Protein

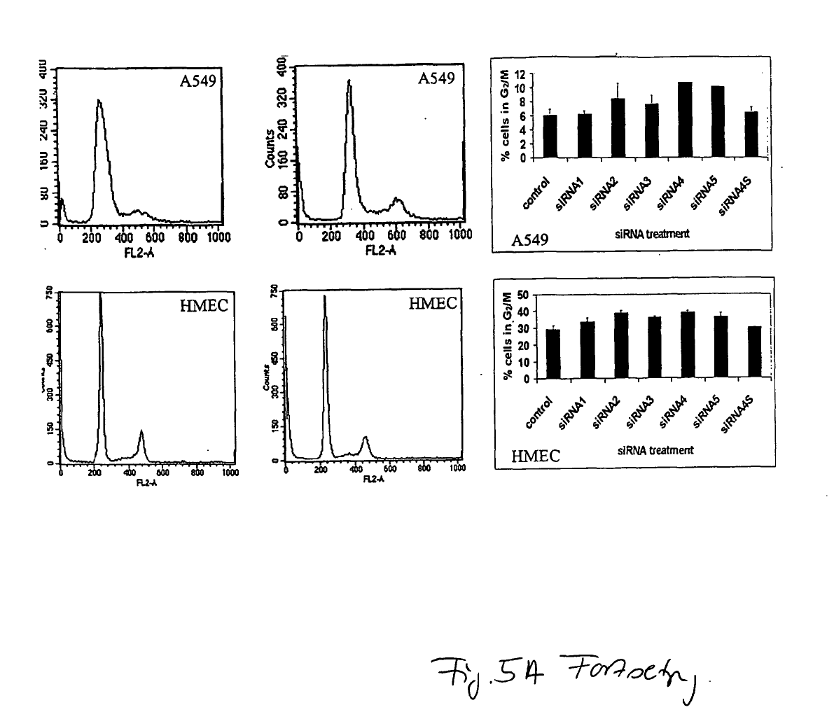

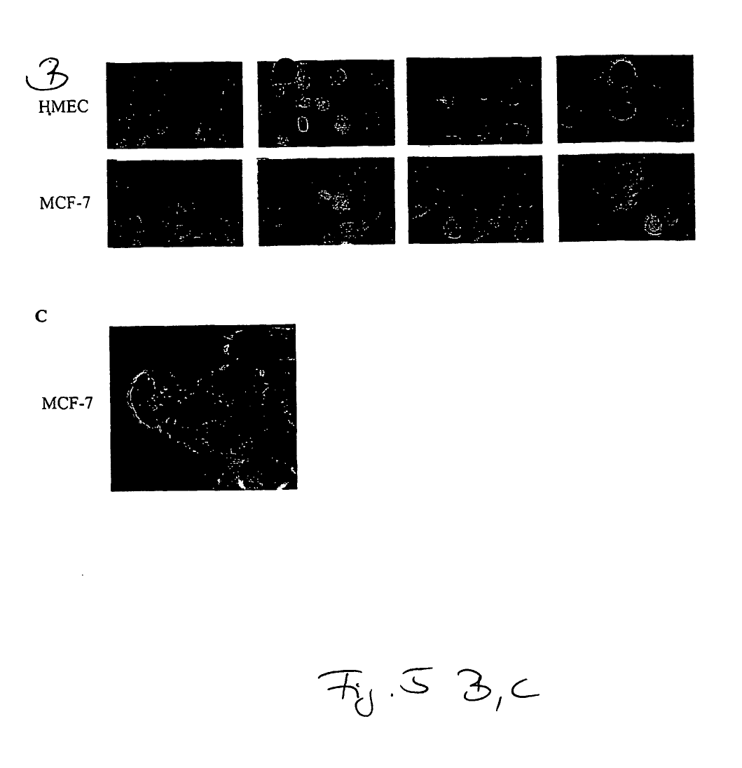

26 phosphorothioate ASOs, each 20 nucleotides in length and predicted to hybridize with human PLK1 mRNA were tested to identify effective candidates capable of inhibiting PLK1 gene expression in human tumor cells. These ASOs were homologues to different regions of PLK1 mRNA, with 9 ASOs targeted to the 5′-untranslated region, 6 targeted to sites within the coding region of PLK1 and 11 targeted to the 3′-untranslated region (FIG. 8a). Binding of ASOs to the complementary sequence of the mRNA for a specific gene results in gradual downregulation of the protein and loss of function of that gene mostly due to the activation of RNase H, which cleaves the mRNA at RNA/DNA duplex sites (Dirksen et al., 1981). Since phosphorothioates are excellent substrates for RNase H, antisense activity for each of these ASOs was evaluated with Northern blot analyses using a PLK1-specific probe.

Treatment of MDA-MB-435 breast cancer cells in vitro with ASOs against PLK1 at a concentration of 250 nM in the presence of uptake-enhancing cationic lipids (DOTAP) led in few cases to an essential loss of PLK1 mRNA within 24 hrs as demonstrated in FIG. 8b. The evaluation of Northern blots standardized to the expression of actin or glyceraldehyde-3phosphate dehydrogenase (G3PDH) mRNA revealed that the ASOs, named P12 and P13, which target the 3′-untranslated domain, are efficient inhibitors for reducing PLK1 mRNA in cultured human MDA-MB-435 cells to levels of 30% and 40%, respectively compared to DOTAP-treated cells. Still, most tested ASOs had limited or no influence on the level of endogenous PLK1 mRNA. Furthermore, two control ASOs (HSV-ASO derived from Herpes Simplex Virus and a nonsense-ASO representing a random sequence) led only to a weak reduction. Specific reduction by the ASOs P12 and P13 could not only be demonstrated for MDA-MB-435 cells but also for other tumor cell types like HeLa S3 and A549 (FIG. 8c, d). ASOs used in the present experiment are summarised in Table I.

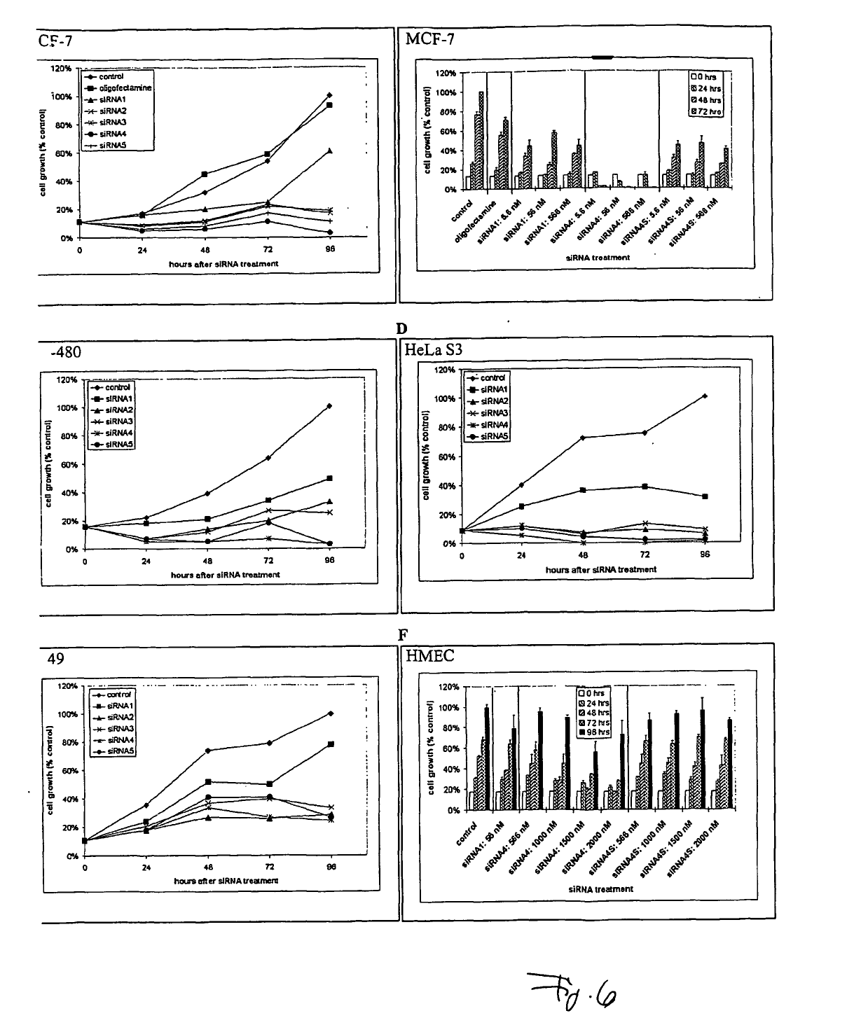

To determine whether decreases in PLK1 mRNA levels induced by ASOs were accompanied by corresponding reduction in protein levels, Western blot analyses were performed to evaluate the expression in different tumor cell lines after single application of ASOs. Significant inhibition (p<0.001) of the 68 kDa-PLK1 protein in MDA-MB-435 cells was observed with the ASOs P12 (80%) and P13 (73%) which were previously shown to be potent inhibitors of gene expression (FIG. 9a). A reduction of PLK1 mRNA induced by the application of P12 and P13 resulted also in a significantly lower level (p<0.05) of the corresponding protein in HeLa S3 (P12: 87%; P13: 47%) and A549 cells (P12: 74%; P13: 61%) (FIG. 9b, c).

Specificity of the ASO action requires that the outcome is dose-dependent and proportional to the downregulation of the gene. To test for specificity, the concentration of P12 and a control ASO (HSV) was correlated to protein levels of PLK1. The treatment of HeLa S3 and MDA-MB-435 cells with P12 exhibited a dose-dependent reduction of PLK1 expression with a median inhibitory concentration (IC50) of 50-75 nM (FIG. 9d, e). The control ASO (HSV) had no significant effect in Western blot analyses. The expression of other cellular proteins such as actin and p38 (MAP kinase family) was not influenced significantly indicating that the effects on cells treated with P12 are sequence-specific for PLK1.

To directly assess whether downregulation of PLK1 protein level correlates also to reduced PLK1 kinase activity, immunoprecipitated PLK1 from HeLa S3 cells was subjected to enzymatic tests. FIG. 9e shows the kinase activity of PLK1 precipitated with immune sera from P12-, control ASO (HSV)- and untreated cells. Although approximately equal amounts of PLK1 protein were present in the three immunoprecipitates, in P12-treated cells phosphorylation of exogenous casein was reduced to 50% of the enzymatic level in control cells. Virtually no kinase activity was detected in the preimmune precipitate (data not shown). These results suggest that the amount of PLK1 protein was specifically reduced in P12-treated cancer cells correlating with low levels of PLK1 activity.

2.2 PLK1-Specific ASOs Inhibit the Growth of Cancer Cells In Vitro

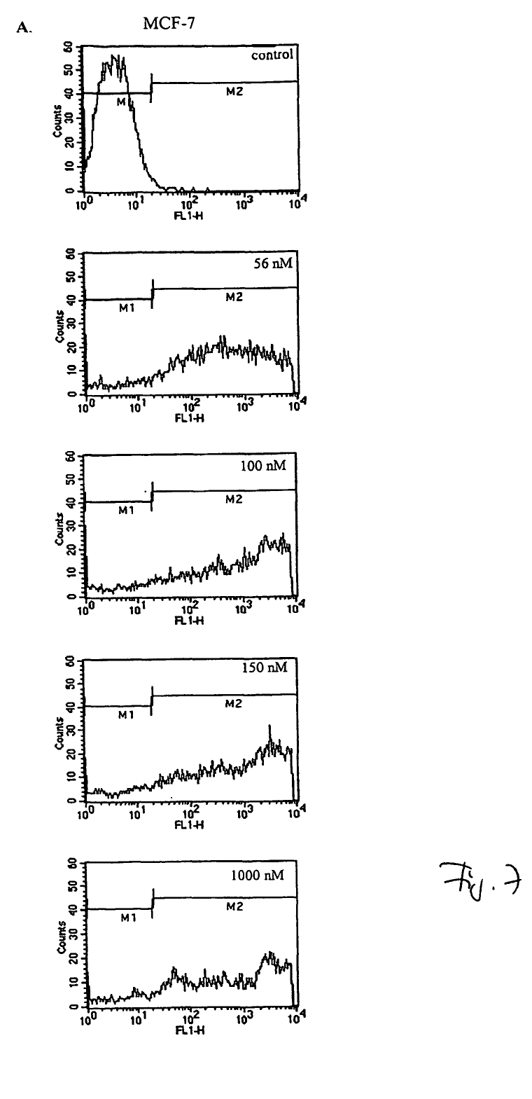

It was of particular interest to determine whether ASOs which reduce PLK1 expression influence the proliferation rate of tumor cells. For this purpose MDA-MB-435 cells were treated once with either P12, P13 or a control ASO (HSV) at a concentration of 250 nM. The growth rate of 5×10⁵ cells was determined over a period of 2 days. The treatment with the lipofection agent (DOTAP) alone or a control ASO (HSV) had only limited effect on the growth rate of MDA-MB-435 cells. In contrast, P12 and P13 had a significant antiproliferative effect (p<0.05) of approximately 98% and 93% compared to DOTAP-treated MDA-MB-435 cells within 48 hrs (FIG. 10a). In addition, the remaining 24 ASOs, which had been tested for their ability to reduce the endogenous level of PLK1 mRNA, were utilized to measure their potential to influence the growth of MDA-MB-435. The analysis revealed that those ASOs, which induced only a slight reduction of cellular mRNA, had only limited inhibitory effect on the proliferation of the breast cancer cell line MDA-MB-435 (data not shown). Whether the reduction of proliferative activity is cell type-specific or can also be achieved in other cancer cell lines was subsequently examined. P12 and P13 were shown to determine the extent of growth inhibition also significantly in HeLa S3 cells (p<0.001) and in A549 cells (p<0.05) suggesting that PLK1 expression is essential for the proliferation of different types of cancer cells (FIG. 10b, c). Additional evidence for the specific activity of the inhibitory ASO P12 came from a dose-dependent reduction of cell growth in MDA-MB-435 and in HeLa S3 cells (FIG. 10d, e). Increasing concentrations of P12 (50-250 nM) reduced the number of MDA-MB-435 cells by 80% and the number of HeLa S3 cells by 95% within 24 hrs compared to control cells. While maximal protein reduction was achieved in HeLa S3 cells with 100 nM (FIG. 9d), a concentration of 250 nM was required for MDA-MB-435 cells. In both cell lines 250 nM were necessary for maximal growth inhibition.

2.3 Reduced Levels of PLK1 Protein Induce Mitotic Abnormalities

To determine whether the death of ASO treated cells was associated with cell cycle arrest or phenotypic abnormalities, we used FACS-analysis and laser scanning microscopy. Flow cytometric analyses of all three cell lines demonstrated that the induction of the G₂/M cell cycle arrest was moderate. The ASO treatment resulted in an increase of cells in G₂/M Of approximately 20-30% (data not shown).

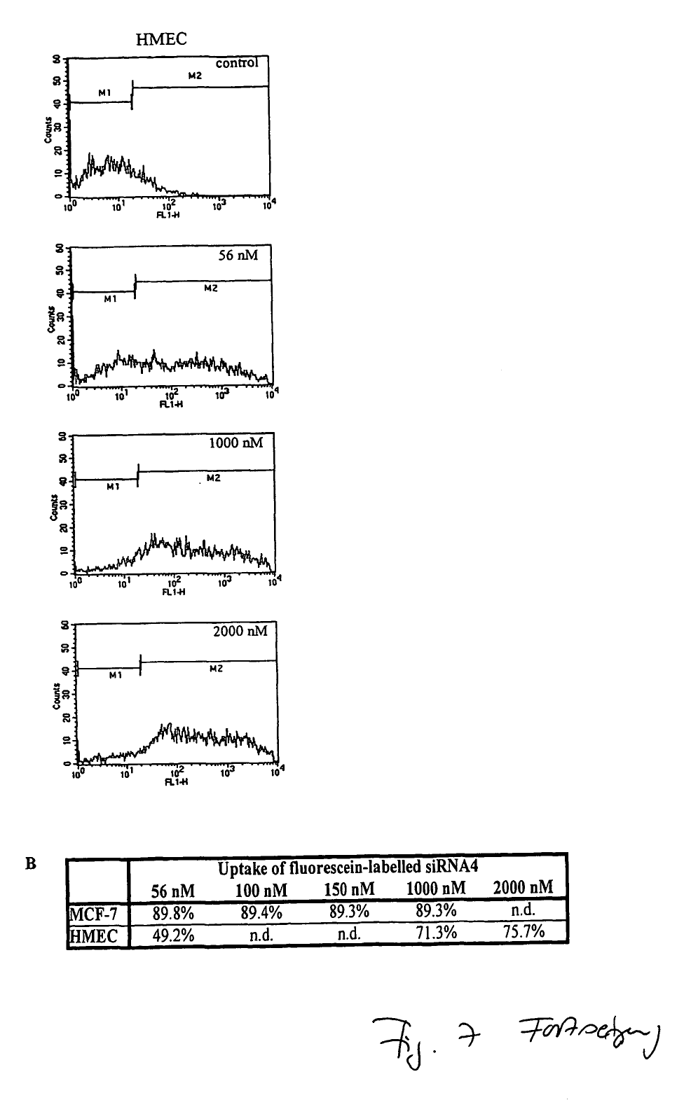

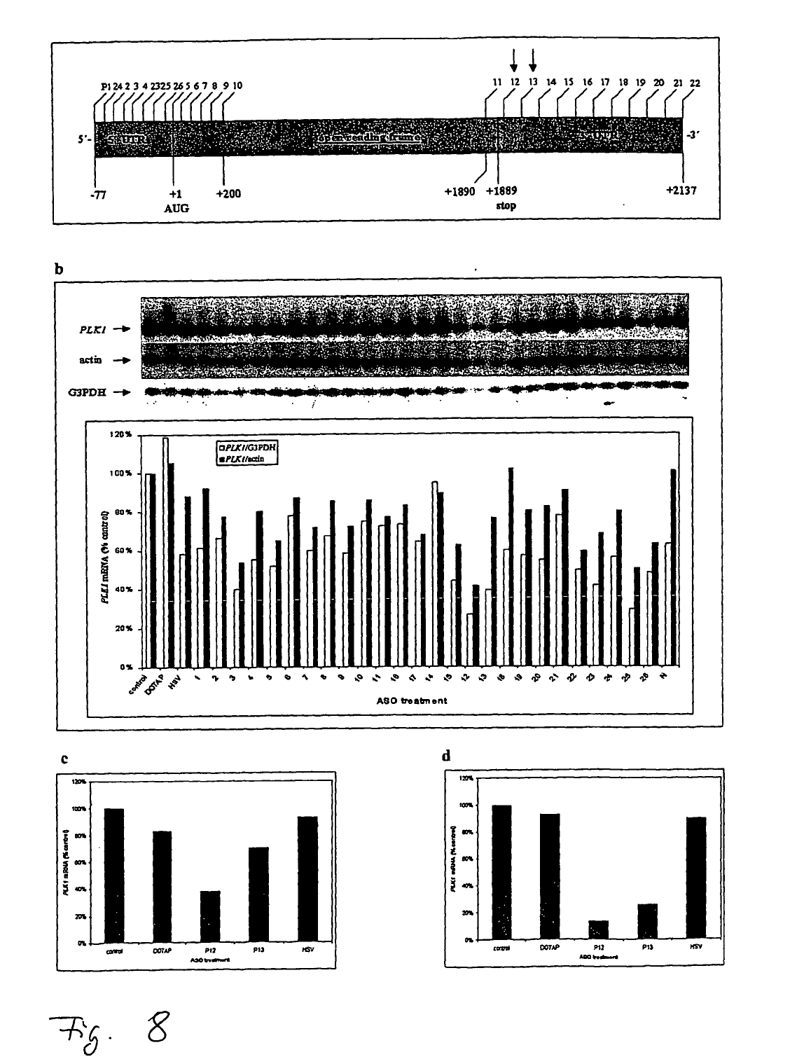

Previous observations have demonstrated that microinjection of PLK1-specific antibodiesresulted in abnormal distribution of condensed chromatin and monoastral microtubule arrays that were nucleated from duplicated but unseparated chromosomes (Lane et al., 1996). Here we showed for the first time the reduction of endogenous PLK1 protein and corresponding PLK1-kinase activity in cancer cells. To analyze cell cycle phenotypes of P12-treated cells under these conditions, we immunolabeled A549 cells. Antibodies directed against α-tubulin to visualize the spindle apparatus and α-tubulin to localize centrosomes were used 48 hrs after transfection with P12 and HSV-ASO, respectively. While most of the HSV-ASO-treated cells proceeded through mitosis without abnormal phenotypes, many cells incubated with P12 gained a rounded shape and lost adherence to the cell culture plastic. Whereas application of HSV-ASOs preserved normal centrosome maturation in A549 lung cancer cells, P12-treated cells displayed often unseparated chromosomes and multiple centrosomes (FIG. 11). Partially separated chromosomes induced the formation of nuclear membranes enclosing at least one centrosome. This led to abnormal distribution of centrosomes in interphase cells as demonstrated in FIG. 11 (a, b).

2.4 In Vivo Antitumor Activity of PLK1 ASOs

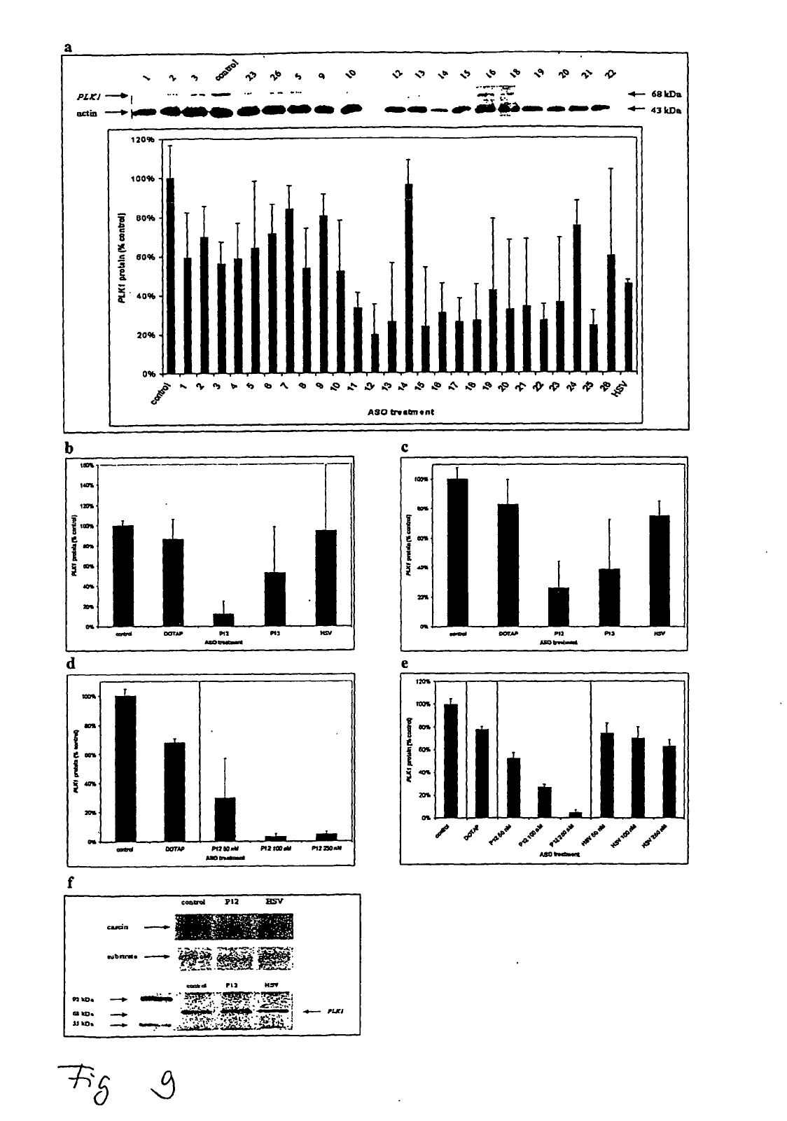

Having demonstrated that PLK1 ASOs are capable of inhibiting growth of cancer cells in culture in a target- and sequence-specific manner, we tested whether P12 and P13 have also an inhibitory capability in vivo. For this purpose tumor fragments derived from a serial passage of three consecutive transplantations of A549 cells were implanted subcutaneously in nude mice. Xenograft mice bearing a tumor of 100 mm³ in volume were treated with 12 mg/kg/day by bolus intravenous injection once daily. The influence of P12 and P13 on the growth of A549 tumors was examined and compared to effects exerted by a control ASO (HSV) or PBS alone. The administration of both ASOs P12 and P13 at a dose of 12 mg/kg/day over a period of 24 days revealed a significant effect on the growth of A549 tumors in mice (FIG. 12a). PLK1 ASOs administered systematically inhibited the growth of A549 tumor xenografts in nude mice by 70% (P12) and 86% (P13), respectively. In contrast, no influence was observed when the tumor-bearing animals were treated with a control ASO (HSV) or PBS alone. The efficacy of ASO treatment on the inhibition of PLK1 expression in tumor cells was confirmed by an immunohistochemical analysis: While administration of control ASOs (12 mg HSV/kg) for a period of 24 days had no significant effect on the frequency of PLK1-positive cells (76%), marked reduction of PLK1 -positive cells (10%) was observed in P12- and P13-treated animals (FIG. 12b). Staining with the conventional marker for cellular proliferation MIB-I revealed a reduction from 61% to 8% of immunostained proliferating cells in tumors.

Different experimental approaches had the goal to define the function of PLK1 in mammalian cells by altering the level of PLK1 expression. First, the inhibition of PLK1 function through antibody microinjection blocked centrosome maturation in both nonimmortalized human Hs68 fibroblasts and HeLa cervical carcinoma cells (Lane et al., 1996). While PLK1-injected HeLa cells revealed severe mitotic defects such as immature centrosomes, nonimmortalized human Hs68 fibroblasts arrested in G₂ suggesting a centrosome-maturation checkpoint sensitive to alterations in PLK1 function. Second, results from transient expression of dominant negative PLK1 differed from previous antibody microinjection experiments in that most of the mitotic HeLa cells were bipolar and cytokinesis seemed to be disrupted (Mundt et al., 1997). Third, adenovirus delivery of a dominant-negative PLK1 induced apoptosis in different tumor cell lines (Cogswell et al., 2000). In contrast, normal human mammary epithelial cells arrested in mitosis, but seemed to escape the loss of centrosome maturation and mitotic defects observed in different cancer cells. Thus, the use of different methods to abrogate the activity of PLK1 revealed its essential role for mitotic progression in mammalian cells. Interestingly, recent data extend previous observations by further showing that the majority of tested tumor lines are more sensitive to the inhibition of PLK1 function than normal epithelial cells (Cogswell et al., 2000). These results suggest that certain tumors show a selective apoptotic response versus normal epithelial counterparts. Still, the methods used in different studies are not suited for the systemic treatment of tumor-bearing animals or cancer patients. Antisense-based methods, which have been proven to inhibit the expression of certain critical cancer genes specifically, have already entered different clinical studies (Agrawal, 1996; Crooke et al., 1994; Zhang et al., 1995). The antisense drug Vitravene has now been approved for the treatment of patients with cytomegalovirus-induced retinitis (Crooke, 1998). Thus, we pursued the goal to test PLK1 as a possible target for cancer therapeutic intervention using an antisense oligonucleotide-based approach by reducing the endogenous level of PLK1 specifically in vitro and in vivo.

In contrast to many chemotherapeutical agents for the treatment of cancer cells, ASOs have the intriguing advantage to act specifically on the gene of interest. To test the specificity of our approach in detail, we analyzed the potential of different PLK1 ASOs to reduce the intracellular level of PLK1 expression and to act in an antiproliferative manner. As expected only very few ASOs were able to reduce the intracellular mRNA level, because due to the three-dimensional structure of mRNA only certain sequence domains are accessible for the heteroduplex formation with ASOs. Two ASOs P12 and P13 targeted against the 3′-untranslated region of human PLK1 mRNA were shown to be potent inhibitors of PLK1 mRNA and protein expression in cell culture. These ASOs displayed IC50 values of 50 nM. Interestingly, our experiments revealed an antisense response, which seemed to reflect the level of endogenous transcripts in the cancer cells examined: For example, the A549 cells with the highest frequency of expression showed only a moderate response. Still, we can not rule out a homeostatic regulation, which can answer a decrease in mRNA concentration with a metabolic compensation. Administration of P12 and P13 to A549-tumor-bearing mice resulted in reduced PLK1 expression and in potent antiproliferative effects.

ASOs represent a potentially powerful method of selectively inhibiting gene expression. However, the study of antisense compounds has been hampered by some parameters resulting in variable experimental results. ASOs and in particular phosphorothioate versions used in many investigations are highly charged macromolecules that can induce toxic effects or might alter cellular functions via association to endogenous proteins which activate non-antisense mechanisms (Kuss et al., 1999). Biological activity attributed to such ASOs may not be the result of the specific inhibition of a target gene. Thus, demonstration of specificity is a key aspect for concluding true antisense action underlying the biological impact of a specific ASO. For this reason we paid considerable attention to the specificity of inhibition of PLK1 gene expression. Several experiments provided evidence for a specific mode of action by ASOs P12 and P13: (i) During an examination of 26 phosphorothioate ASOs targeted to different sites within the PLK1 mRNA, only a few candidates were potent inhibitors of PLK1 expression. (ii) Control ASOs (HSV-derived, nonsense) had no significant effect on PLK1 mRNA and protein expression in cell culture and in Xenograft experiments. (iii) Our data revealed that the suppression of PLK1 by ASOs was dose-dependent. In addition, we could demonstrate that the expression of other cellular genes like actin and p38 was not affected by P12 or P13 treatment.

The number of antisense cancer drugs that have entered clinical trials is increasing. At least four of these compounds are currently in phase II trials including those targeting protein kinase C-alpha, bcl-2, c-raf and H-ras (Cunningham et al., 2000; Nemunaitis et al., 1999; O'Dwyer et al., 1999; Waters et al., 2000; Yuen et al., 1999). Various studies suggest that the inhibition of early steps in signaltransduction causes the simultaneous activation of alternative, parallel signaling pathways resulting also in cellular proliferation. Thus, ASOs targeted against c-raf kinase led only to a transient inhibition of cellular growth (Monia et al., 1996). This effect might be due to the turnover of applied ASOs or due to the location of the raf protein in the signaling cascade. In contrast to early steps in signaltransduction represented by c-raf, ras, PKC, which are targets of antisense approaches, PLK1 is the first example of a kinase triggering terminal steps in the signaling cascade. Multiple observations provided evidence for a central role of PLK1 in the mitotic progression in mammalian cells which can not be by-passed on alternative routes of signal transduction (Cogswell et al., 2000; Lane et al., 1996; Mundt et al., 1997). Moreover, inhibition of PLK1-function was shown to induce tumor-selective apoptosis compared to normal epithelial cells. Considering these observations our experimental data suggest PLK1 as new target for cancer treatment. In addition to phosphorothioate ASOs, modified derivatives with altered sugar moiety or backbone which have improved pharmacokinetical and toxicological properties or low molecular weight inhibitors targeted against human PLK1 need to be developed for future therapeutic endeavors.

2.5 Oligonucleotides and Antibodies

Phosphorothioate oligonucleotides (ASOs) were synthesized and purified by MWG Biotech (Ebersberg). Monoclonal PLK1-antibodies were obtained from Transduction Laboratories (Heidelberg) for Western blots and from Zytomed (San Francisco, USA) for kinase assays. Antibodies for actin were purchased from Sigma (Deisenhofen) and for p38 from Santa Cruz Biotechnologies (Heidelberg).

2.6 RNA Preparation and Northern Blot Hybridization

For the isolation of total RNA an RNeasy mini-kit was used according to the manufacturer s protocol (Qiagen, Hilden). Radiolabeling of antisense strands for PLK1 and β-actin was performed using 100-250 μCi of [α-³²P]dCTP (6000 Ci/mmol) for each reaction, 50 μM of each other dNTP and 10 pmol (each) of primer PLK1 -17-low (5′-tgatgttggcacctgccttcagc-3′) corresponding to position 1533-1554 within the open reading frame of PLK1 or actin-2-low (5′-catgaggtagtcagtcaggtc-3′) as described previously (Wolf et al., 1997). Probes corresponding to aa 285-497 of PLK1 were generated by PCR. Northern blotting and hybridizations were carried out as reported (Wolf et al., 1997).

2.7 Cell Culture

DMEM and DMEM/F-12 1:1 mixture were obtained from Sigma, Ham's F12 and FCS from PAA (Linz, Österreich). PBS, Opti-MEM, glutamine, penicillin/streptomycin and trypsin were purchased from Invitrogen. Tumor cell lines MDA-MB-435 and A549 were obtained from CLS (Heidelberg), HeLa S3 from DSMZ (Braunschweig) and cultured according to their instructions with slight modifications.

2.8 Western Blot Analysis

48 hrs after ASO treatment cells were lysed for subsequent Western blotting (Hock et al., 1998). Membranes were kept for 1 hr with monoclonal PLK1 antibodies (1:250) and monoclonal actin antibodies (1:200.000) followed by incubation with goat anti-mouse antibodies (1:2.000) for 30 min. Western blots were performed as described (Bohme et al., 1996).

2.9 Kinase Assays

Cells were lysed 24 hrs after ASO treatment for determination of PLK1 kinase activity. Endogenous PLK1 was immunoprecipitated using monoclonal PLK1 antibodies (Zytomed) and then incubated with 0.5-1 μg of substrate and 2 μCi of [α³²-P]ATP for 30 min at 37° C. in kinase buffer (20 mM HEPES pH 7.4, 150 mM KCl, 10 MM MgCl₂, 1 mM EGTA, 0.5 mM DTT, 5 mM NaF, 0.1 mM Na₃VO₄). Kinase reaction was stopped and fractionated on a 12% SDS-polyacrylamide gel (BioRad, München).

2.10 Indirect Immunofluorescence

Cells were stained as described before (Holtrich et al., 2000). Antibodies were used as follows: monoclonal α-tubulin (Cedarlane, Canada) 1:100, polyclonal rabbit PLK1 (Yuan et al., 1997) 1:100 or monoclonal γ-tubulin (Sigma, Deisenhofen) 1:100.

2.11 In Vitro Application of ASOs and Determination of Cell Proliferation

In vitro-inhibitory activities of ASOs on cell proliferation were tested by transfection with different ASOs. Transfections were performed using the DOTAP method (Roche, Mannheim). In brief, 1 day before transfection cells were split to a density of 5×10⁵/25 cm²-culture flask corresponding to a density of 40-50% at the time of transfection. Cells were treated with ASOs at a concentration of 250 nM. After 3,5 hrs incubation with ASOs in Opti-MEM with DOTAP at 37° C., transfection mix was replaced by normal culture media. Cell numbers were determined at the appropriate time after ASO treatment by direct counting using a hemacytometer. Cell viability was assessed by trypan blue staining. Each experiment was performed at least in triplicate and the standard deviation for each group was determined.

2.12 Tumor Xenograft Studies

Human cancer xenograft models were established with at least 3 independent groups of 5 athymic nude mice (nu/nu) NMRI 8-10 weeks old (Harlan Winkelmann). For this purpose A549 cells were harvested, washed with PBS, resuspended in normal culture media and then 2×10⁶ cells were injected subcutanously into the animals flank regions. Arising tumors were serially passaged by a minimum of three consecutive transplantations before the start of treatment. Then tumor fragments were implanted subcutaneously in both flanks of the nude mice and ASO application was started 25 days after transplantation when the tumor reached a volume of 100 mm³. ASO treatment (formulated in PBS) was carried out daily by bolus injection (100 μl) into the animals tail vein at a dose of 12 mg/kg body weight/day for 24 days. Tumor diameters were determined using a caliper. Volumes were calculated according to the formula V=π/6×largest diameter×smallest diameter². Standard deviations were calculated. Experiments were carried out in triplicate. After sacrificing the animals tumors were excised for immunohistochemistry (Yuan et al., 1997).

2.13 Immunohistochemistry

Tumor sections of Xenograft tumors were prepared as described (Yuan et al., 1997). Slides were incubated with monoclonal MIB-I antibodies (1:10; Dianova, Hamburg) and a polyclonal PLK1 antibody (1:200; Transduction Laboratories, Heidelberg). As detection system for PLK1 the EnVision+System (Dako, Hamburg) was used according to the manufacturer's protocol.

2.14 Quantitative Analysis

For semiquantitative analysis of Northern and Western blot signal intensities the autoradiographs were scanned using a Kodak gel documentation system (ID 3.5). After integration of signal intensities expression of PLK1 and actin were correlated for quantitative comparison. The expression values were given in percentage of control. For quantitative evaluation of immunohistochernistry in A549 tumors, 10 high power magnification fields (100×) per slide were analyzed. Immunoreactive staining of PLK1 and MIB-I is given in percent positive tumor cells.

### EXAMPLE 3

Inhibition of PLK1 Activity by Inhibitory Peptides

3.1 Synthesis of Peptides

Peptides were synthesized according to Fmoc synthesis protocols with double or triple coupling reactions using TBTU as activator on a Symphony synthesizer (Rainin Instrument Co, Woburn, Mass., USA). Purifications were performed by RP-HPLC on a Waters (Milford, Mass., USA) Delta-Pak C18 column with a Waters liquid chromatography system. Quality control was performed by analytical RP-HPLC using a Waters Alliance 2690 separation module equipped with a Waters 996 photodiode array detector and by MALDI-TOF mass spectrometry. Peptide sequences:

P1 (polo-box (aa 410-429) linked to a 16-mer carrier from Antennapedia): H2N-WVSKWVDYSDKYGLGYQLCDRQIKIWFQNRRMKWKK-COOH

P2 (mutated polo-box (aa 410-429) linked to a 16-mer carrier from Antennapedia):

H2N-WVSKFADYSDKYGLGYQACDRQIKIWFQNRRMKWKK-COOH

P3 (16-mer carrier from Antennapedia as a control): H2N-RQIKIWFQNRRMKWKK-COOH

3.2 Cell Culture and Growth Inhibition Assays.

Cancer cell lines HeLa S3 (cervix), MCF-7 (breast) and Saos-2 (osteosarcoma) were grown at 37° C. in 5% CO₂ in Ham's F 12, RPMI 1640 and McCoy's 5a medium, respectively containing 10% FBS, 2 mM L-glutamine. To assay for growth inhibition, exponentially growing cells (0.3-2.5×10⁴) were seeded into 24-well plates. On the following day cells were incubated with peptides at varying concentrations without serum for 3 h followed by addition of complete medium. On day 3, 6 and 8 cells were treated again and harvested on day 3, 6, 8 and 10 to determine cell numbers using a Hemacytometer. Cell viability was assessed by trypan blue staining. Each experiment was repeated at least three times.

3.3 Flow Cytometry and Indirect Immunofluorescence Staining.

Cell cycle analysis was performed using a Cycle TEST™ PLUS DNA reagent kit (Becton Dickinson) according to the manufacturer's instructions. Briefly, cells were washed with PBS, treated with RNase A and stained with propidium iodide (PI). The analysis was performed using a Becton Dickinson FACScan flow cytometer. Using the MODFIT LT 2.0 software (Verity Software House, Topsham, Me.) for each individual sample 30,000 cells were analyzed.

For staining cells were grown in slide flasks and treated with peptides for 24 h. Then, cells were fixed in 4% paraformaldehyde for 30 min, permeabilized in 0.2% Triton X-100 for 20 min and stained with α-tubulin (Serotec/Biozol, Eching) 1:100, polyclonal rabbit PLK1 1:100, or monoclonal α-tubulin (Sigma) 1:100. Stained cells were analyzed with a confocal laser scan microscope (CLSM) or a fluorescence microscope.

3.4 Annexin V Apoptosis Assay.

Cells were seeded into 6-well plates, allowed to attach overnight, and then treated with different peptides at a concentration of 10 μM. Cells were trypsinized after 16 h and incubated with Annexin V according to the manufacturer's recommendations (Mo Bi Tech).

3.5 In Vitro Kinase Assays

PLK1 purified from Sf 9 cells was incubated with 0.5-1 μg of PLK1-specific substrates and 2 μCi of [α-³²P] ATP for 20 min at 37° C. in kinase buffer (20 mM HEPES pH 7.4, 150 mM KCl, 10 mM MgCl₂, 1 mM EGTA, 0.5 mM DTT, 5 mM NaF, 0.1 mM Na₃VO₄ and 100 μM ATP).

3.6 The Polo-Box Fused to an Antennapedia Carrier Translocates to the Cytoplasm and Nucleus of Cancer Cells.

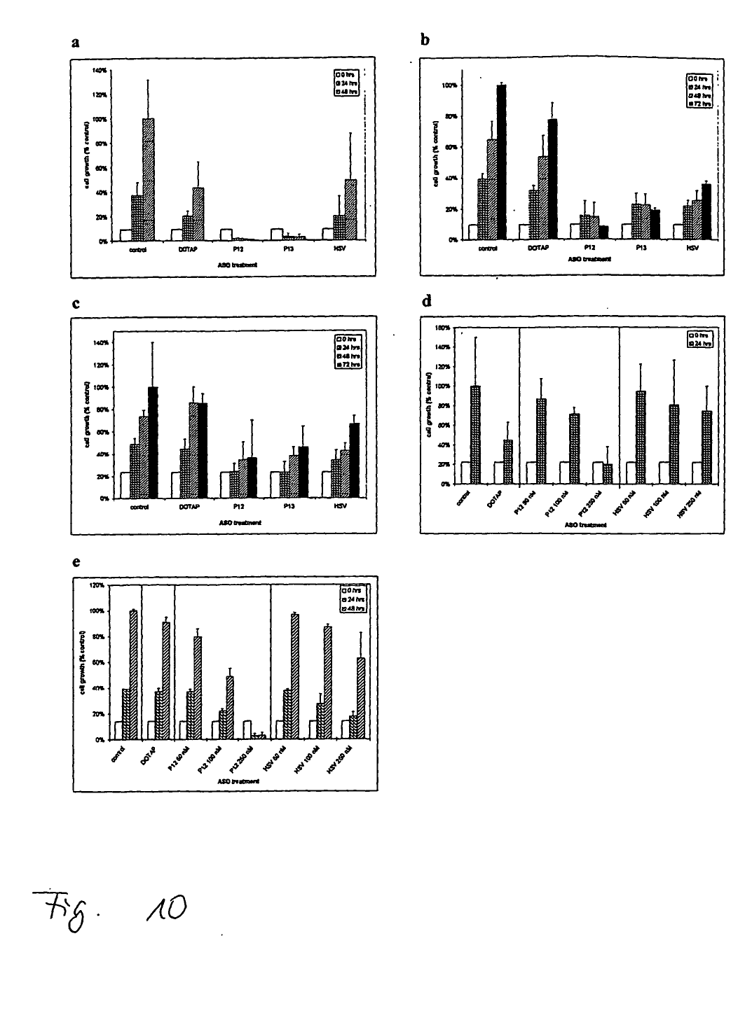

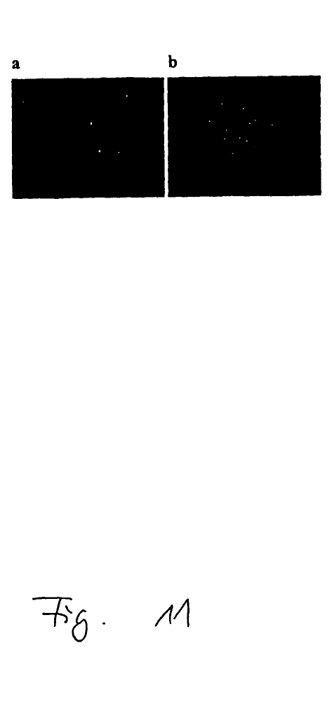

PLK1 was shown to be of importance for the G₂/M transition (Glover et al. 1998 and Nigg et al. 1998). The polo-box, a highly conserved domain of PLK1 (aa 410-439), contributes to binding of substrates as well as for its correct subcellular localization (Lee et al. 1999 and May et al. 2002). We assumed that peptides representing the polo-box might be able to compete with PLK1 for endogenous substrates and docking proteins. Thus, we tested polo-box-specific peptides for their ability to suppress the function of endogenous PLK1. In previous experiments an Antennapedia homeodomain sequence was linked to peptides and allowed the resulting chimerical peptides to be transported across the cell membrane directly from the cell culture medium to both, cytoplasm and nuclear compartment (Derossi et al. 1996). In our study we linked the Antennapedia homeodomain (16 aa) to the core region of the polo-box derived from PLK1 (aa 410-429) (FIG. 13A) or to a mutated version (Lee et al. 1999) to find out whether fusion peptides could enter tumor cells from the culture medium. The transport kinetics revealed that FITC-labelled peptides reached the cytoplasm within 15 min and then emerged immediately to the nucleus. After 2 h peptides entered 98-100% of cells in culture. Both fusion peptides (wild-type P1 and mutant P2) were efficiently delivered into the cytoplasm and nucleus of MCF-7 (FIG. 13B: e, f), HeLa S3 cells (FIG. 13B: g, h) and Saos-2 cells (data not shown). Peptides were distributed diffusely in the cytoplasm and accumulated at the nuclear membrane. Elevated concentrations of peptides were detected at nucleoli. The overall distribution and uptake of both peptides (wild-type P1 and mutant P2) in HeLa S3 and MCF-7 cells was similar. 4 h after peptide-treatment morphological changes in MCF-7 and HeLa S3 cells due to toxic effects were not observed (FIG. 13B: a-d). FITC-labeled peptides were still detectable after 24 h (data not shown).

3.7 Wild-Type Polo-Box P1 Inhibits the Proliferation of human MCF-7, Saos-2 and HeLa S3 Cells.

At first the human breast cancer cell line MCF-7 was tested to assay effects exerted by polo-box-specific peptides on its proliferation. A dose kinetics was established to choose an appropriate working concentration for the treatment of cells. While the wild-type peptide P1 at concentrations between 0.01 and 0.5 μM did not affect the growth behavior, concentrations above 1 μM started to inhibit the proliferation of MCF-7 cells (FIG. 13C). Inhibition of proliferation occurred in a dose-dependent manner in the range between 1-10 μM of P1. A general toxic effect was observed above 20 μM of peptide P1 (data not shown).

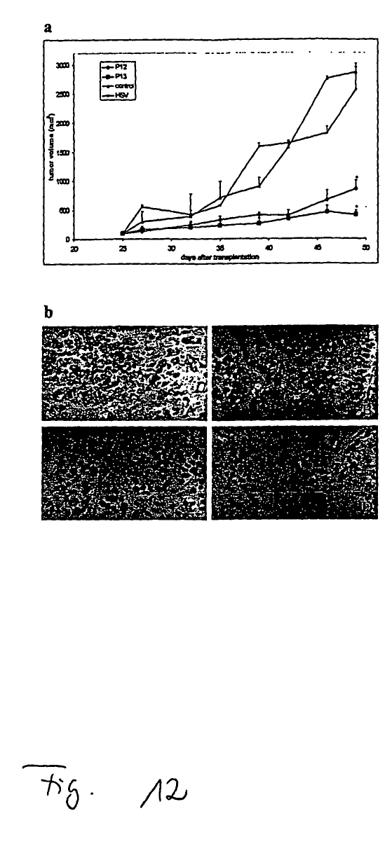

To investigate whether antiproliferative effects exerted by polo-box peptides are restricted to certain cell-types, we tested different human cancer lines: MCF-7 (breast), Saos-2 (osteosarcoma) and HeLa S3 (cervix). As shown in FIG. 14A-C the wild-type polo-box (P1) exerted an inhibitory effect on cell proliferation which was most prominent in MCF-7 cells. Interestingly, the mutated polo-box (P2), which differed from P1 at three positions, reduced the antiproliferative potency of P1 significantly. While an intermediate effect was seen for Saos-2 cells, little inhibition occurred in HeLa S3 cells. The control 16-mer-carrier P3 alone had eventually no effect at concentrations between 5-10 μM. Thus, the results suggested that the inhibitory effect exerted by polo-box peptides is sequence-specific. In addition, testing of primary epithelial cells (HMEC) revealed that the polo-box P1 inhibited proliferation of HMEC, but to a much lower extent than MCF-7 cells.

3.8 Treatment of Cancer Cells with Polo-Box-Specific Peptides Induces G₂/M Cell Cycle Arrest.

Since the functional down-regulation of PLK1 by microinjection of PLK1-specific antibodies or overexpression of a dominant-negative PLK1 induced G₂/M arrest (Lane et al. 1996 and Cogswell et al. 2000), we investigated the impact of polo-box-specific peptides on the cell cycle of tumor cells. FIG. 14D depicts a representative analysis of HeLa S3 cells at day 6. Incubation with the wild-type polo-box P1 induced an increase of cells in G₂/M by 25-35% compared to control cells. We decided further to determine the subpopulation of mitotic cells by microscopical means. The percentage of mitotic cells increased 3-4 fold after wild-type polo-box (P1) treatment (14.7%) compared to control cells (3.5%) (FIG. 14E). In contrast, the control peptide P3 had no effect. The mutated polo-box P2 exhibited an intermediate inhibitory potential. These data suggest that polo-box-specific peptides have the ability to induce cell cycle arrest at G₂/M.

3.9 Polo-Box-Specific Peptides Induce Apoptosis in Cancer Cells.

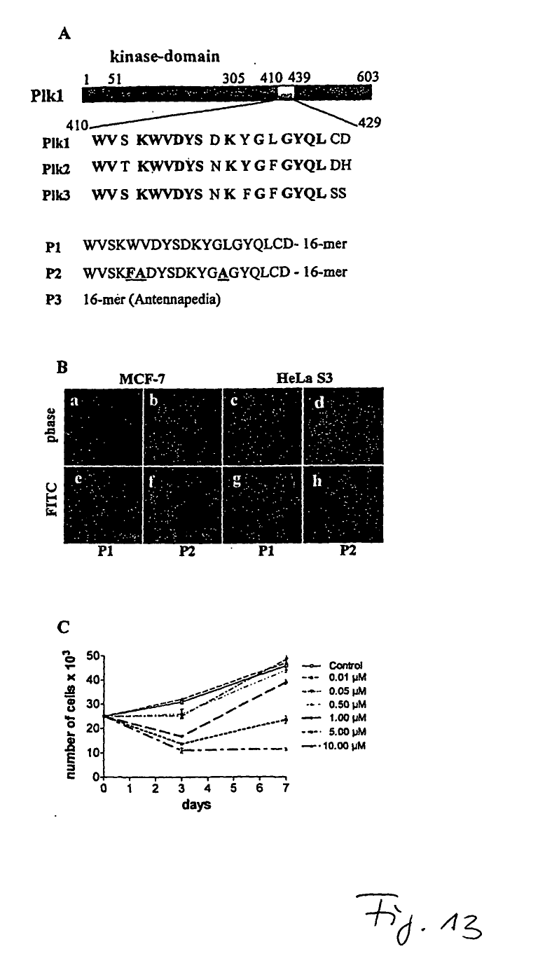

Cells treated with peptides were labeled with annexin V biotin and P1 to determine the extent of cellular apoptosis. The treatment with wild-type polo-box PI induced an elevated percentage of apoptotic cells including early phase apoptosis (annexin V-positive) and late phase apoptosis (annexin V- and PI-positive) compared to the control peptide P3 (FIG. 15A, B). To further confirm the finding, we stained the DNA and analyzed the apoptotic phenotype by fluorescence microscopy. In MCF-7 and HeLa S3 cells the typical apoptotic morphology was observed including condensation and fragmentation of nuclear chromatin, shrinkage of the cytoplasm and loss of membrane asymmetry (FIG. 15C: b, d, e, g, i, j). In contrast, no significant increase of apoptotic cells was found in carrier peptide (P3)-treated cells (FIG. 15C: c, h) and non-treated control cells (FIG. 15C: a, f). In cells incubated with the mutated form P2, there was also increased apoptosis, but less extensive compared to the treatment with wild-type polo-box (data not shown).

3.10 Wild-Type Polo-Box Induces Abnormal Mitotic Phenotypes with Misaligned Chromosomes and Multiple Spindle Poles.

Polo-box-treated cells were further analyzed using DNA- and α-tubulin-staining to monitor spindle apparatus and chromosomal figures. Many treated cells showed multiple (FIG. 15D: d, f) or monoastral spindle poles (FIG. 15D: e). Furthermore, chromosomes in P1-treated HeLa S3 cells were misaligned, not properly segregated and partially condensed (FIG. 15D: a-c).

3.11 The Polo-Box Inhibits the phosphorylation of Substrates by PLK1 In Vitro.

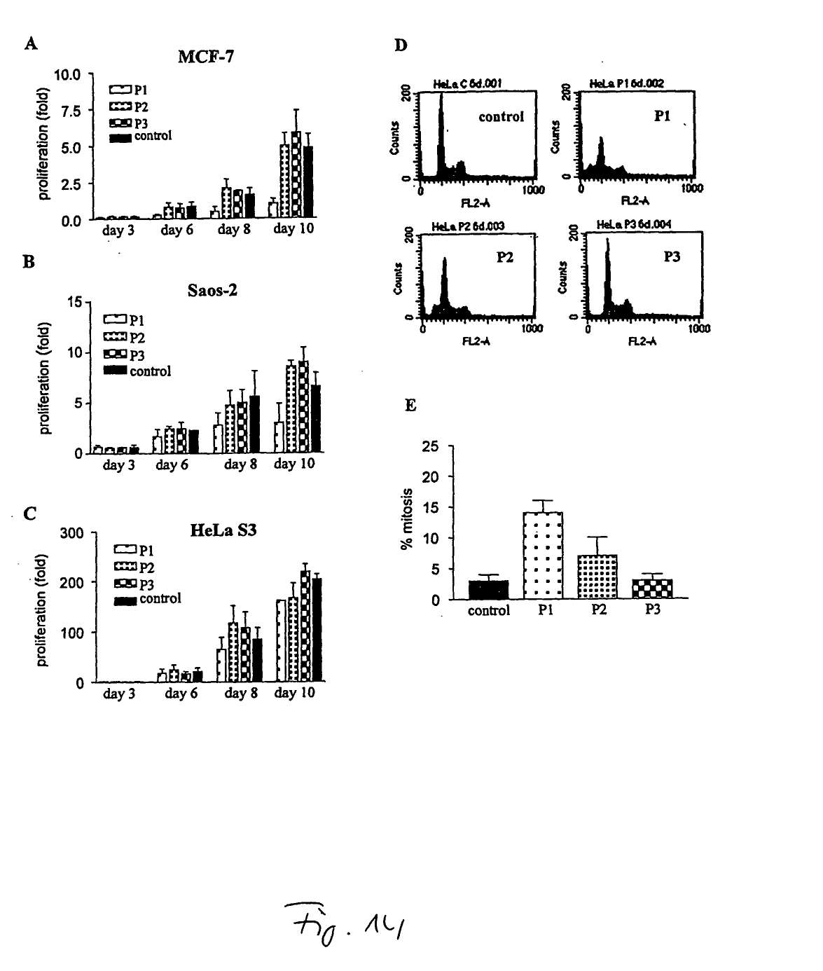

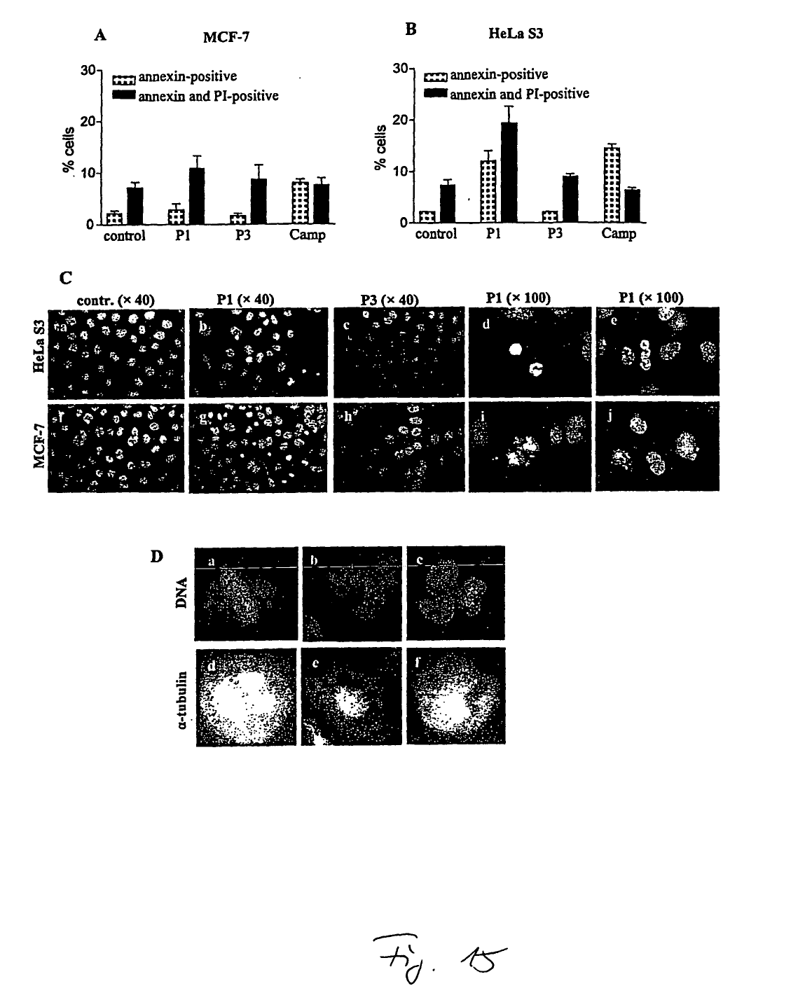

Furthermore, we studied potential mechanisms underlying the inhibitory effect on cell proliferation by the polo-box (P1). For this purpose kinase assays using specific substrates of PLK1 were performed (FIG. 16). Whereas the polo-box P1 and its mutated form P2 decreased the phosphorylation of substrates clearly, the carrier peptide P3 exhibited only at high concentrations of 25-50 μM a weak inhibition of phosphorylation. Within a dose range of 6.25 to 25.0 μM the inhibitory effect of the polo-box P1 differs from that of the mutated form P2: wild-type polo-box induced at least two fold more inhibition than its mutated form. In contrast, the carrier P3 showed no effect at all. Below 1.56 μM, only the wild-type polo-box still exhibited some effect. The data suggest that the inhibitory effect of the polo-box could at least partially be due to a reduction of PLK1-specific phosphorylation.

The Antennapedia homeodomain corresponding to the third helix of the DNA binding domain of a Drosophila transcription factor is internalized into eukaryotic cells by a receptor-independent process (Derossi et al. 1996). The internalization peptide has been used as vector for small peptides derived from c-myc, p21 and p16 to traverse the cell membrane (Giorello et al. 1998, Fahraeus et al. 1998 and Mutoh et al. 1999). We linked this carrier to a peptide (P1) representing the polo-box of PLK1 or to the mutated polo-box (P2). Fused peptides entered the cells quickly and efficiently. Both polo-box peptides P1 and P2 were found in the cytoplasm and nucleus of cancer cells. No difference in import efficiency between the wild-type polo-box P1 and its mutated form P2 could be detected in HeLa S3, MCF-7 and Saos-2 cells. Toxic side effects were not observed in cell lines at concentrations between 0.01-10 μM.

### EXAMPLE 4:

Cancer Inhibition in Nude Mice after Systemic Application of U6 Promoter-Driven siRNAs Targeted Against PLK

4.1 Plasmid Sequences

Plasmids were constructed with the pBS/U6 vector (Hannon 2002) using standard techniques. To generate an intermediate plasmid for the subsequent steps of cloning hairpin RNAs (shRNAs) targeted to PLK1, a 21-nt oligonucleotide (5′GGCGGCTTTGCCAAGTGCTTA-3′) annealed with a 25-nt oligonucleotide (5′AGCTTAAGCACTTGGCAAAGCCGCC-3′) corresponding to siRNA2 (Spänkuch-Schmitt, 2002) was first inserted into the pBS/U6 vector digested with Apal (blunted) and HindIII. The inverted motif, which contains a 6-nt spacer and a termination string of five thymidine residues (5′AGCTTAAGCACTTGGCAAAGCCGCCCTTTTTG-3′, 5′AATTCAAAAAGGGCGGCTTTGCCAAGTGCTTA-3′), was then subcloned into the HindIII and EcoRI sites of the intermediate plasmid to generate pBS/U6/shRNA/PLK1. For cloning of the scrambled shRNA, referred to as PLK1S, the same protocol was applied.

4.2 Isolation and Purification of Plasmids.

Plasmids pBS/U6/shRNA/PLK1, pBS/U6/shRNA/PLK1S and pBS/U6 were produced and released after quality control by PlasmidFactory contract DNA manufacturing service (PlasmidFactory, Bielefeld, Germany) in endotoxin free “Research Grade” quality. In brief, manufacturing of plasmid DNA was divided into two major phases. The first phase starts with the transformation of wellcharacterized E. coli DH5\acute{a} host cells with fully characterized plasmids. The resulting transformed bacteria were checked carefully for the expected characteristics. Subsequently, plasmid-producing cells were transferred to the cultivation process. This requires the generation of a cell bank, which is essential for reproducible large-scale cultivation of bacterial biomass. Cultivation of bacteria was performed without using antibiotics in order to avoid additional analytical studies demonstrating that that purified plasmid DNA is free of contaminating antibiotics. In addition, no animal-derived substances were used in cultivation media.

After the bacterial biomass was subjected to QC tests for product contents and absence of contaminating DNA it was processed by alkaline lysis for release of plasmids from bacteria. The resulting lysate was separated from insoluble matter and cell debris. Subsequently, the plasmid molecules were separated chromatographically from soluble biomolecules (such as host chromosomal DNA, RNA, nucleotides, lipids, residual proteins, amino acids, saccharides and endotoxins), as well as salts and other buffer components. Using this protocol more than 90% of the isolated plasmid DNA was shown to be the covalently closed circular form. DNA solutions were adjusted to a concentration of 1.0 mg/mL in PBS buffer (phosphate buffered saline) and aliquoted in DNA storage vials at volumes of 200 μL and labeled. Each vial was frozen and stored at −20° C. until use.

Aurintricarboxylic acid (ATA) was obtained from Sigma-Aldrich (Taufkirchen, Germany). A stock solution of ATA at a concentration of 1 mg/ml was sterilized by filtration and stored at 4° C.

4.3 Cell Culture.

Ham's F12 and fetal calf serum (FCS) were purchased from PAA Laboratories (Colbe, Germany). Phosphate buffered saline (PBS), Opti-MEM I, glutamine, penicillin/streptomycin, and trypsin were from Invitrogen (Karlsruhe, Germany). FUGENE™6 was from Roche (Mannheim, Germany). The tumor cell line HeLa S3 (cervix) was obtained from DSMZ (Braunschweig, Germany) and cultivation was performed according to the supplier's instructions.

4.4 In Vitro Transfection with the Parental Vector pBS/U6 or with Plasmids Expressing shRNAs Against PLK1 (shRNA/PLK1) and its Scrambled Control (shRNA/PLK1S).

Cells were transfected with plasmids using the FuGENE™6 protocol (Roche, Mannheim, Germany). In brief, 1 day prior to transfection cells were seeded without antibiotics at a density of 2×10⁵ cells per 10 cm² cell culture dish corresponding to a density of approximately 50% at the time of transfection. The amount of plasmid ranged between 3.0 μg and 6.0 μg plasmid per 10 cm² culture dish. Control cells were incubated with culture medium without plasmid. The recombinant vector pBS/U6/shRNA/PLK1S and the parental vector pBS/U6 served to control for the specificity of shRNA-mediated effects.

Additional experiments were performed using the nuclease inhibitor ATA and plasmid at a ratio of 1:5 (ATA:DNA) to test whether plasmid stability enhanced by the addition of ATA influences shRNA-mediated effects in HeLa S3 cells. Plasmid and ATA were mixed and then added to the Opti-MEM I/FuGENE™6 mix. Just before transfection cells were covered with normal culture medium and the transfection mix (containing Opti-MEM I, FuGENE™6, and plasmids, with or without ATA) was added. Following incubation of cells at 37° C. for at least 4 hours to overnight, fresh culture medium was supplemented to a final volume of 2 ml/10 cm². Cells were harvested 72 hours after the beginning of the transfection period for the analysis of mRNA expression. All transfections were performed in triplicate.

4.5 RNA Preparation and Northern Blots.

Total RNA was isolated using RNeasy mini-kits according to the manufacturer's protocol (Qiagen, Hilden, Germany). Probes for Northern blots were generated by radiolabeling antisense strands for PLK1 and β-actin using 100 μCi-200 μCi of [α-³²P]dCTP (6000 Ci/mmol) for each reaction, 50 μM of each of the other dNTPs, and 10 pmol of either primer PLK1-17-low (5′-TGATGTTGGCACCTGCCTTCAGC-3′), corresponding to position 1533-1554 within the open reading frame of PLK1, or actin-2-low (5′-CATGAGGTAGTCAGTCAGGTC-3′), as described previously (Holtrich, 1994). Northern blotting and hybridizations were carried out as described before (Holtrich, 1994). All blots from in vitro experiments were reprobed with β-actin probes in order to compare actin-normalized PLK1 mRNA levels. Ethidium bromide staining of rRNAs (18S and 28S) was used to normalize PLK1 mRNA levels for Northern blots from in vivo experiments.

In Northern blotting experiments PLK1-, β-actin-expression and ethidium bromide staining (18S and 28S rRNAs) were quantified using a Kodak gel documentation system (ID 3.5). Integration of signal intensities from scanned autoradiographs and gels was followed by quantitative comparison of PLK1 and actin expression or by quantitative comparison of PLK1 expression and ethidium bromide staining; i.e., the ratio of PLK1 and actin signals was determined for each in vitro treatment, and the ratio of PLK1 expression and ethidium bromide staining of 18S and 28S rRNAs was determined for the in vivo experiment. Values are given in percentage of levels in control cells for in vitro experiments and as percentage of levels in pBS/U6-treated mice for in vivo testing.

4.6 Isolation of Plasmid DNA from Murine Blood.

All animal experiments were approved by the Regierungspräsidium Darmstadt and performed in certified labs of the School of Medicine Frankfurt. Blood samples were collected from the vena cava of mice under Enfluran anesthesia (Abbott, Wiesbaden, Germany). One ml of blood was immediately mixed with 1.6 mg EDTA. Plasmid DNA was incubated for 5 min to 4 hours with blood from nude mice to control for the stability of DNA dependent on different ratios of DNA:ATA (50:1, 5:1 and 0.5:1). Subsequently, total DNA was isolated using QIAamp DNA Mini-kits according to the manufacturer's protocol (Qiagen, Hilden, Germany) to determine ex vivo the stability of U6 promoter-containing vectors in murine blood (see below). DNA was separated using 1% agarose gels. Plasmids were visualized by ethidium bromide staining on a UV transilluminator.

4.7 Southern Blot Analysis.

To determine the effect of ATA on the stability of plasmids, total DNA was isolated from murine blood and electrophoresed as described above. To depurinize and denature DNA, gels were incubated first for 15 min in 0.25 M HCl on a shaker to induce doublestrand breaks and next for 30 min under denaturing conditions (1.5 M NaCl and 0.5 M NaOH). Subsequently gels were incubated twice for 15 min in neutralizing solution (1.5 M NaCl, 0.5 M Tris-HCl, pH 7.2, and 1 mM EDTA, pH 8.0). DNA was then transferred onto nylon membranes as deseribed for Northern blotting analysis. Membranes were dried at room temperature and DNA was fixed on membranes for 5 min using a UV transilluminator. For detection of the plasmid pBS/U6/shRNA/PLK1 a probe was generated by radiolabeling antisense strands for PLK1 using for each reaction 100 μCi-200 μCi of [α-³²P]dCTP (6000 Ci/mmol), 50 μM of each of the other dNTPs, and 10 pmol of primer PLK1-150-as (5′-GCAGCAGAGACTTAGGCACAA-3′), corresponding to position 310-330 within the open reading frame of PLK1, as described previously (Holtrich, 1994). The blots were prehybridized for 20 min at 68° C. in QuickHyb™ (Stratagene, Amsterdam, The Netherlands) and hybridized in fresh QuickHyb™ containing probes at 1×10⁶ cpm/ml for 1 hour at 68° C. Membranes were washed twice in 2×SSC for 15 min at 36° C. and exposed to MP Hyperfilms (Amersham Pharmacia Biotech., Freiburg, Germany).

4.8 Isolation of Plasmid DNA from Tumors for PCR-Based Detection.

To evaluate the transfection in xenograft experiments, total DNA was isolated from tumors the day after the last treatment using QIAamp DNA Mini-kits according to the manufacturer's protocol (Qiagen, Hilden, Germany. Parental and recombinant plasmids were analyzed in total DNA with PCR using plasmid-specific primers pBS-500s (5′-GAATAGACCGAGATAGGGTTGAGT-3′) and pBS-500as (5CGTCGTTTTACAACGTCGTGACTG-3′) derived from the parental vector pBS/U6. PCR products were separated using 1% agarose gels, stained with ethidium bromide, and visualized on a UV transilluminator.

4.9 In Vivo Treatment of Nude Mice with shRNA-Expressing Plasmids.

Human cancer xenograft models were established with at least 3 independent groups of 5 athymic nude mice (nu/nu) NMRI 8-10 weeks old. For this purpose HeLa S3 cells were harvested, washed with PBS and resuspended in normal culture media. Thereafter 2×10⁶ cells were injected subcutaneously into the animals' flank regions. Tumors that developed were serially passaged by a minimum of three consecutive transplantations prior to treatment. Then tumor fragments were implanted subcutaneously in both flanks of each nude mouse tested. Treatment with plasmids was started when tumors reached a volume of 100 mm³. Administration of plasmid was carried out 3 times a week (Monday, Wednesday, Friday) by injection of 500 μl PBS containing 10 μg plasmid with or without 2 μg ATA (ATA:DNA=1:5) into the tail vein. The first group was treated with the plasmid pBS/U6/shRNA/PLK1 without ATA, the second group with a combination of the plasmid pBS/U6/shRNA/PLK1 and ATA, the third group received the vector expressing the scrambled version pBS/U6/shRNA/PLK1S and ATA, and the fourth group was treated with the parental vector pBS/U6 with ATA.

Tumor diameters were determined using a caliper. Volumes were calculated according to the formula V=π/6×largest diameter×smallest diameter². Experiments were carried out in triplicate, and means and 95% confidence intervals (Cl) were calculated. After the animals were sacrificed, tumors were excised for determination of PLK1 mRNA using Northern blot analysis and to perform immunohistochemistry.

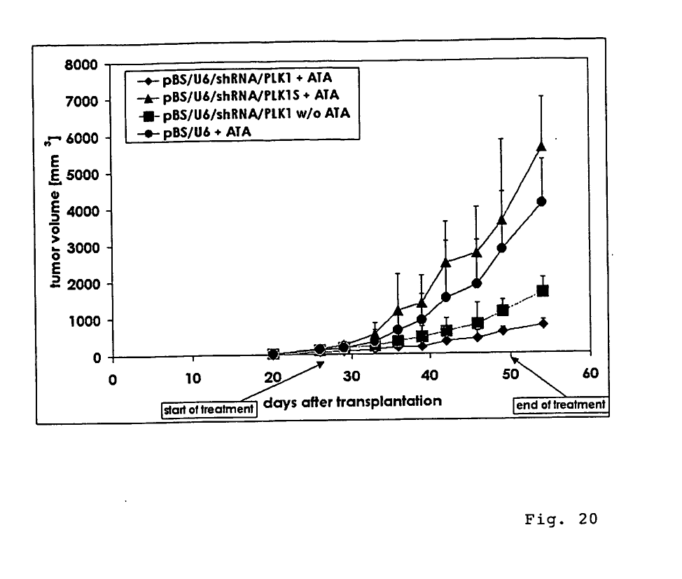

A further experiment was conducted using the same plasmids as described above to treat nude mice bearing A549 tumours (lung carcinomas). The results are shown in FIG. 20.

4.10 Immunohistochemistry.

Immunohistochemical analysis of xenograft tumors was performed as described previously (Yuan, 1997). Slides were incubated with polyclonal rabbit anti-human Ki-67 antibodies (1:30, Dako, Hamburg, Germany) or with monoclonal PLK1 antibodies (1:600, Transduction Laboratories, Heidelberg, Germany). The EnVision+System (Dako, Hamburg, Germany) was used as a detection system according to the supplier's protocol.

4.11 Statistical Methods.

Each Northern blot experiment was performed 3 times. Means of signal intensities normalized to actin or means of signal intensities normalized to ethidium bromide staining of rRNAs were calculated. Statistical analysis was performed with two-way ANOVA (GraphPad Prism, GraphPad Software, Inc., San Diego, Calif.) to consider random effects of individual gels and different treatments. For two-way ANOVAs all treatment groups were compared to pBS/U6-treated mice (in vivo), and for Northern blots using HeLa S3 cells (in vitro) all transfected cells were compared to control cells. P values and 95% Cl for the statistical significance of the changes caused by each treatment (in vitro and in vivo) are given.

4.11 Results.

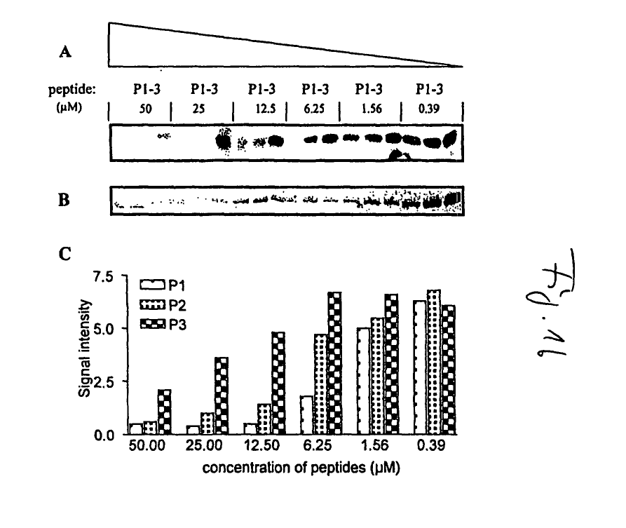

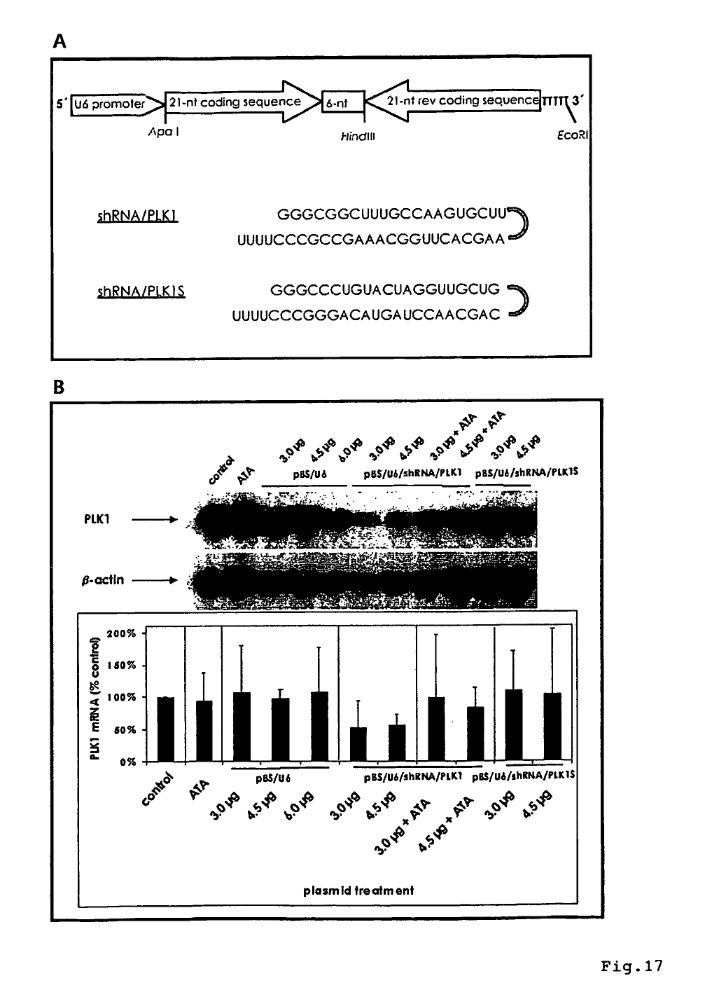

Experimental introduction of sIRNA duplexes 21-nt in length into mammalian cells is now widely used to disrupt the activity of cellular genes homologous in sequence to the transfected siRNA (Elbashir et al. 2001). Although siRNAs are effective tools for inhibition of gene function in mammalian cells, their suppressive effect is of limited duration. The use of short hairpin RNAs (shRNAs) has been found to be an alternative strategy that could bypass such limitations and provide a tool for evoking stable suppression. In designing constructs for shRNAs, several investigators have taken advantage of RNA polymerase III promoters, which use exclusively non-transcribed promoter sequences and have well defined initiation and termination sites producing various small RNA species (Paul et al. 2002, Sui et al. 2002, Yu et al. 1995). Beyond these, the U6 snRNA promoter and the H1 RNA promoter have been well characterized (Chong et al. 2001, Hannon et al. 1991, Lobo et al. 1990). In the investigations leading to the present invention the human U6 snRNA promoter was selected to express shRNAs for efficient ‘knock-down’ of PLK1 gene function in mammalian cells to prolong effects exerted by PLK1-specific synthetic siRNAs. DNA templates (pBS/U6/shRNA/PLK1 and pBS/U6/shRNA/PLK1S) were generated for the synthesis of shRNAs that correspond to the recently described siRNA2 (shRNA/PLK1) that was shown to inhibit PLK1 expression in HeLa S3 cells efficiently and a scrambled version of siRNA2 (shRNA/PLK1S) (Spänkuch-Schmitt et al. 2002). Each construct produces a shRNA composed of two sequences 21-nt in length in an inverted orientation separated by a spacer of 6-nt in length and followed by a termination signal of 5 thymidine nucleotides for PolIII (FIG. 17A).

The constructs were initially tested in HeLa S3 cells to evaluate the specificity of the effects of the U6 promoter-driven shRNAs on PLK1 gene expression in vitro. The impact of both shRNAs (shRNA/PLK1, shRNA/PLK1S) on PLK1 mRNA was determined 72 hours after the beginning of transfection. Treatment of HeLa S3 cells with shRNA/PLK1 expressed by pBS/U6/shRNA/PLK1 caused a significant reduction in PLK1 mRNA levels to 52% with 3.0 μg pBS/U6/shRNA/PLK1 (p<0.05) and to 56% with 4.5 μg pBS/U6/shRNA/PLK1 (p<0.01) compared to control cells (FIG. 17B). Assuming a transfection efficiency between 40% and 60% for plasmid pBS/U6/shRNA/PLK1 in cell culture, expression of shRNA/PLK1 ‘knocks-down’ PLK1 mRNA levels very efficiently. No effect on PLK1 mRNA expression was observed in cells treated with the vector expressing the scrambled control pBS/U6/shRNA/PLK1S or the non-recombinant vector pBS/U6 at any of the plasmid concentrations tested.

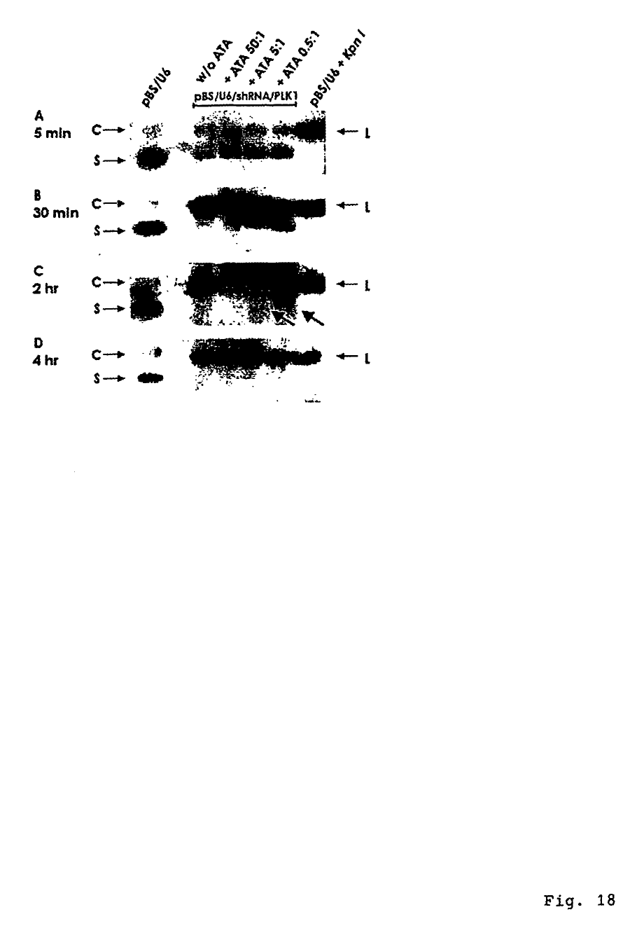

A potential barrier to the successful transfection of foreign DNA into mammalian cells in vivo is the activity of blood-borne nucleases. The hypothesis was tested that the stability of plasmid DNA in murine blood can be improved in the presence of a specific nuclease inhibitor such as aurintricarboxylic acid (ATA), previously shown to inhibit DNase I, RNase A, S1 nuclease, exonuclease III and various endonucleases (Blumenthal and Landers 1973, Hallick et al. 1977). Ex vivo plasmid degradation assays using peripheral blood from nude mice were performed to assess the effects of the inhibitor ATA on blood-derived nuclease activity. Mass of DNA, volume of peripheral blood and temperature were kept constant for each incubation time to examine the influence of nuclease activity on plasmid integrity by Southern blot experiments. When pure plasmid DNA (pBS/U6/shRNA/PLK1 w/o ATA) was incubated in murine blood, most of the supercoiled plasmid disappeared within 5-30 min, but corresponding degradation products (circular and linear forms) were detectable up to 4 hours (FIG. 18). Different ratios of plasmid DNA:ATA were tested. When the plasmid was co-administered with ATA at ratios of DNA:ATA=50:1, 5:1, 0.5:1, the stability of the supercoiled form was evidently increased (FIG. 18). While the supercoiled form was still clearly visible after 2 hours at a ratio of DNA:ATA=0.5:1, the supercoiled form was at the limit of detection at a ratio of 5:1 (FIG. 18, panel C, indicated by arrows). The

Southern blot analysis revealed that the corresponding degradation products (circular and linear forms) were detectable in murine blood for more than 4 hours (FIG. 18, panel D). Thus, the stability of the degradation products appeared to be less dependent on the presence of ATA than did the stability of the supercoiled form (FIG. 18, panel D).

When increasing amounts of ATA were added to plasmid DNA in the blood of different mice, DNA degradation was decreased in all samples. Addition of ATA also protected the integrity of U6 promoter-containing vectors in human blood: After an incubation time of 4 hours at 37° C. stabilization of supercoiled form was increased from 75% at a ratio of ATA:DNA=1:50 to 92% at a ratio of ATA:DNA=1:0.5 compared to the signal intensity of a defined quantity of supercoiled plasmid (data not shown). These data demonstrate the existence of significant levels of nuclease activity in mammalian blood, consistent with the hypothesis that extracellular nucleases contribute to clearance of plasmids.

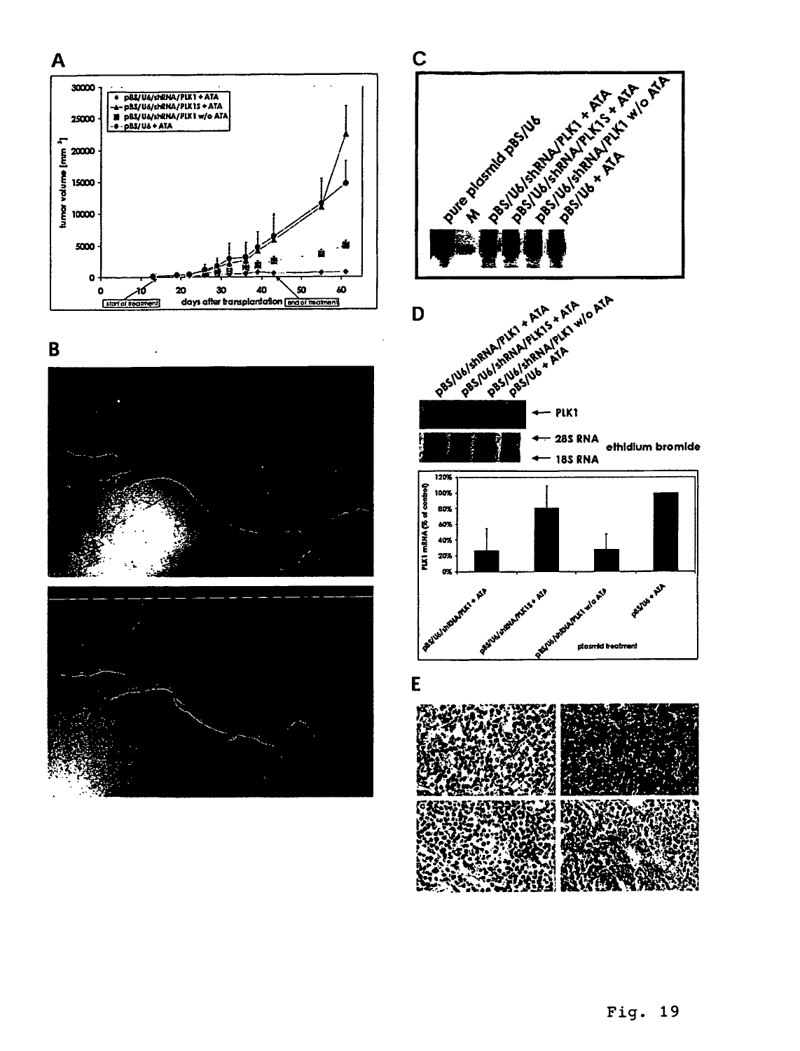

To evaluate whether improved stability of plasmid pBS/U6/shRNA/PLK1 ‘knocks-down’ PLK1 gene expression in vivo, experiments with subcutaneously implanted tumor xenografts (HeLa S3 cells) in nude mice were performed. In the xenograft experiments mice were treated for 26 days and then observed for 4 weeks. Application of plasmids with or without ATA was started 16 days following tumor fragment implantation, when tumors reached a volume of 50-100 mm³. Plasmids pBS/U6/shRNA/PLK1, pBS/U6/shRNA/PLK1S or pBS/U6 in 0.5 ml of phosphate buffered saline solution (PBS) with or without ATA were each administered to tumor-bearing mice by bolus intravenous injection three times a week for 26 days. In initial experiments, pBS/U6/shRNA/PLK1 was tested three times a week at a dose of 0.33-0.4 mg/kg body weight and the impact was compared with that of the same dose of pBS/U6/shRNA/PLK1S expressing the scrambled version as a control. Administration of shRNA/PLK1-expressing plasmids (pBS/U6/shRNA/PLK1) displayed significant antiproliferative effects on the growth of HeLa S3 tumors in mice, whereas tumor growth was not inhibited by the control plasmids pBS/U6/shRNA/PLK1S compared to tumor growth in pBS/U6-treated animals (FIG. 19A, B). Co-injection of pBS/U6/shRNA/PLK1 and ATA at a ratio of 5:1 was more efficient in tumor inhibition compared to pure plasmid pBS/U6/shRNA/PLK1 without ATA: Administration of pBS/U6/shRNA/PLK1 in the presence of ATA reduced tumor growth to 13% of the growth seen after administration of the control vector pBS/U6 (p<0.01); by contrast, pBS/U6/shRNA/PLK1 in the absence of ATA reduced tumor growth to only 32% of the control growth (p<0.05). Thus, addition of ATA was very effective in increasing the inhibitory effect of shRNA/PLK1 in the tumor xenografts. No reduction in body weight was seen following treatment of nude mice with pBS/U6/shRNA/PLK1 or pBS/U6/shRNA/PLK1S with or without ATA at a dose of 0.33-0.4 mg/kg. Interestingly, resumption of tumor growth was not observed during the 4 week-period after termination of the therapy.

In addition, it was studied whether ATA could also augment the inhibitory effect of U6 promoter-driven expression of shRNAs targeted against PLK1 in transfection experiments with HeLa S3 cells in vitro. The addition of ATA to the liposome-mediated transfection of plasmid DNA (pBS/U6/shRNA/PLK1) did not improve the inhibitory effect of shRNA/PLK1 significantly in cell culture experiments, which is likely due to efficient protection of plasmid DNA against nucleases by liposomes alone (data not shown).

Total DNA from xenograft tumors was isolated to analyze in more detail the transfection of plasmid DNA in vivo. A 500-bp fragment was generated in PCR reactions using plasmid (pBS/U6)-specific primers and tumor DNA from animals treated with pBS/U6, pBS/U6/shRNA/PLK1 (with or without ATA) and pBS/U6/shRNA/PLK1S demonstrating the capability of pBS/U6 and its derivates to penetrate the membrane of tumor cells in vivo (FIG. 19C).

To evaluate the effect of pBS/U6/shPNA/PLK1 on PLK1 expression in tumor cells, we prepared total RNA from tumors of mice treated for 26 days with plasmid pBS/U6, pBS/U6/shRNA/PLK1 or pBS/U6/shRNA/PLK1S, and we analyzed PLK1 mRNA levels by Northern blots. Administration of pBS/U6/shRNA/PLK1 to mice resulted in suppression of PLK1 mRNA levels in tumors (FIG. 19D). pBS/U6/shRNA/PLK1 administered with or without ATA reduced PLK1 mRNA expression to 27% or 28%, respectively, of the PLK1 mRNA level following administration of the control vector pBS/U6 (p<0.01 in both cases). By contrast, the level of PLK1 expression in mice treated with the scrambled control vector pBS/U6/shRNA/PLK1S was not suppressed. To determine whether decreases in PLK1 mRNA levels in tumor tissues induced by shRNA/PLK1 also translate to a reduction in proteins levels, an immunohistochemical study was performed (FIG. 19E). In animals treated with the control vector pBS/U6 89% of tumor cells were PLK1-positive, compared to 12% of tumor cells in pBS/U6/shRNA/PLK1-treated mice (FIG. 19E, panels a and b). In addition, the growth rate of tumors from different treatment groups was assessed by immunohistochemical evaluation of Ki-67 as a common marker of cellular proliferation. In contrast to animals treated with pBS7U6/shRNA/PLK1 without ATA, pBS/U6/shRNA/PLK1S and pBS/U6 levels of Ki-67 antigen in tumors from animals treated with pBS/U6/shRNA/PLK1 with ATA were reduced mice (FIG. 19E, panels c and d). The results of the Ki-67 immunostaining indicate that the antineoplastic effects observed for pBS/U6/shRNA/PLK1 with ATA result from marked inhibition of cellular proliferation in HeLa S3 tumor cells.

While the feasibility and potential of siRNA as general means for the treatment of cancer has not been demonstrated yet, previous investigations revealed that siRNA from chemical synthesis or from exogenously administered plasmid can inhibit transgenes such as luciferase or GFP in adult mice (Brummelkamp et al. 2002, Lewis et al. 2002, McCaffrey 2002, Xia et al. 2002). Experimental evidence for the inhibition of an endogenous gene by siRNA has been reported to our knowledge only for β-glucuronidase in mouse liver, following injection of adenovirus particles through the tail vein of adult mice (Xia et al. 2002). However, systemically administered adenovirus vectors can provoke immune responses that have been shown to limit the effectiveness of peripheral gene transfer (Vorburger and Hunt 2002). Although the potential of viral vector-mediated gene transfer is considered superior to non-viral delivery of DNA for gene-transfer, in vivo gene transfer with naked DNA is reproducible, simple and safe. Our results with the nuclease inhibitor ATA suggest that transfection strategies for pure plasmid DNA-mediated gene transfer might be reconsidered for in vivo administration. This is in line with previous observations demonstrating that DNA transfection of macaque, murine and human respiratory tissue can be enhanced by adding ATA (Glasspool-Malone et al. 2002). Data on systemic application of ATA are limited. Still, the effect of ATA on platelet aggregation in baboons was investigated by intravenous infusion to screen for future potential in xenograft rejection models (Alwayn et al. 2000). While a dose of 24 mg ATA/kg/day decreased platelet aggregation and increased coagulation time in baboons, 12 mg ATA/kg/day retained normal blood parameters. Since in our experiments 80 μg ATA/kg were infused together with plasmid DNA only three times a week, thrombotic disorders are less likely.

We describe a powerful, novel strategy for the efficient suppression of tumor growth in nude mice. We demonstrate for the first time that U6 promoter-driven hairpin RNAs targeted against PLK1 suppress tumor growth in mice when administered systemically by intravenous iniection in a solution stabilized by addition of the nuclease inhibitor ATA. The combination of hairpin-mediated silencing with effective in vivo gene delivery strategies generates a long-lasting silencing signal that allows for therapeutic approaches based on stable RNA interference in humans.

### References

Agrawal S. (1996). Trends Biotechnol., 14, 376-387.

Agrawal, S., Jiang, Z., Zhao, Q., Shaw, D., Cai, Q., Roskey, A., Channavajjala, L., Saxinger, C., Zhang, R. 1997. Mixed-backbone oligonucleotides as second generation antisense oligonucleotides: in vitro and in vivo studies. Proc. Natl. Acad. Sci. U.S.A. 94, 2620-2625.

Alexandru G, Uhlmann F, Mechtler K, Poupart M A, and Nasmyth K. Phosphorylation of the cohesin subunit Sccl by Polo/Cdc5 kinase regulates sister chromatid separation in yeast. Cell 2001; 105:459-472.

I. P. Alwayn et al., Xenotransplantation. 7, 247-257 (2000).

Amarzguioui, M., Holen, T., Babaie, E., Prydz, H. 2003. Tolerance for mutations and chemical modifications in a siRNA. Nucleic Acids Res. 31, 589-595.

T. Blumenthal and T. A. Landers, Biochem.Biophys.Res.Commun. 55, 680-688 (1973).

Bohme B, VandenBos T, Cerretti D P, Park L S, Holtrich U, Rubsamen-Waigmann H, and Strebhardt K. (1996). J Biol. Chem., 271 24747-24752.

Braasch, D. A., Corey, D. R. 2002. Novel antisense and peptide nucleic acid strategies for controlling gene expression. Biochemistry 41, 4503-4510.

Brinkley B R. Managing the centrosome numbers game: from chaos to stability in cancer cell division. Trends Cell Biol 2001; 11: 18-21.

T. Brummelkamp, R. Bernards, R. Agami, Cancer Cell 2, 243 (2002).

Byers B and Goetsch L. (1974). Cold Spring Harb. Symp. Quant. Biol., 38, 123-131.

Chiu, Y. L., Rana, T. M. 2002. RNAi in human cells: basic structural and functional features of small interfering RNA. Mol. Cell 549-561.

S. S. Chong, P. Hu, N. Hemandez, J Biol. Chem. 276, 20727-20734 (200 1).

Clay F J, McEwen S J, Bertoncello I, Wilks A F, and Dunn A R. (1993). Proc.Natl.Acad.Sci. US.A, 90, 4882-4886.

Cogswell J P, Brown C E, Bisi J E, and Neill S D. Dominant-negative polo-like kinase I induces mitotic catastrophe independent of cdc25C function. Cell Growth Differ 2000; 11:615-623.

Conn, C. W., Hennigan, R. F., Dai, W., Sanchez, Y. and Stambrook, P. J. Incomplete cytokinesis and induction of apoptosis by overexpression of the mammalian polo-like kinase, Plk3. Cancer Res., 60: 6826-6831, 2000.

Crooke S T, Grillone L R, Tendolkar A, Garrett A, Fratkin M J, Leeds J, and Barr W H. (1994). Clin. Pharmacol. Ther., 56, 641-646.

Crooke S T. (1998). Antisense Nucleic Acid Drug Dev., 8, vii-viii.

Crooke S T. (2000). Oncogene, 19, 6651-6659.

Cunningham C C, HoWund J T, Schiller J H, Geary R S, Kwoh T J, Dorr A, and Nemunaitis J. (2000). Clin. Cancer Res., 6, 1626-1631.

Der S D, Yang Y L, Weissmann C, and Williams B R. A double-stranded RNA-activated protein kinasedependent pathway mediating stress-induced apoptosis. Proc Natl Acad Sci U S A 1997;94:3279-3283.

Derossi, D., Calvet, S., Trembleau, A., Brunissen, A., Chassaing, G. and Porchiantz, A. Cell internalization of the third helix of the Antennapedia homeodomain is receptor-independent. J. Biol. Chem., 271: 18188-18193, 1996.

Descombes, P. and Nigg, E. A. The polo-like kinase Plx1 is required for M phase exit and destruction of mitotic regulators in Xenopus egg extracts. EMBO J., 17: 1328-1335, 1998.

Dirksen M L and Crouch R J. (1981). J Biol. Chem., 256, 11569-11573. do Carmo A M, Tavares A, and Glover D M. Polo kinase and Asp are needed to promote the mitotic organizing activity of centrosomes. Nat Cell Biol 200 1;3:421-424.

Doxsey S J. Re-evaluating centrosome function. Nat Rev 2001 ;2:688-698.

Elbashir S M, Harborth J, Lendeckel W, Yalcin A, Weber K, and Tuschl T. Duplexes of 21-nucleotide RNAs mediate RNA interference in cultured mammalian cells. Nature 2001 ;411:494-498.

Elbashir, S. M., Martinez, J., Patkaniowska, A., Lendeckel, W., Tuschl, T. 2001b. Functional anatomy of siRNAs for mediating efficient RNAi in Drosophila melanogaster embryo lysate. EMBO J 20, 6877-6888.

Fahraeus, R., Lain, S., Ball, K. L. and Lane, D. P. Characterization of the cyclin-dependent kinase inhibitory domain of the INK4 family as a model for a synthetic tumor suppressor molecule. Oncogene, 16: 587-596, 1998.

Fire A, Xu S, Montgomery M K, Kostas S A, Driver S E, and Mello C C. Potent and specific genetic interference by double-stranded RNA in Caenorhabditis elegans. Nature 1998;391 :806-811.

Fire A. RNA-triggered gene silencing. Trends Genet 1999; 15:358-363.

Giorello, L., Clerico, L., Pescarolo, M. P., Vikhanskaya, F., Salmona, M., Colella, G., Bruno, S., Mancuso, T., Bagnasco, L., Russo, P. and Parodi, S. Inhibition of cancer cell growth and c-Myc transcriptional activity by a c-Myc helix 1-type peptide fused to an internalization sequence. Cancer Res., 58: 3654-3659, 1998.

J. Glasspool-Malone et al., J Gene Med. 4, 323-2 (2002).

Glover D M, Hagan I M, and Tavares A A. Polo-like kinases: a team that plays throughout mitosis. Genes Dev 1998;12:3777-3787.

Golsteyn R M, Schultz S J, Bartek J, Ziemiecki A, Ried T, and Nigg E A. Cell cycle analysis and chromosomal localization of human PLK1, a putative homologue of the mitotic kinases Drosophila polo and Saccharomyces cerevisiae Cdc5. J Cell Sci 1994; 107 (Pt 6):1509-1517.

R. B. Hallick, B. K. Chelm, P. W. Gray, E. M. Orozco, Jr., Nucleic Acids Res. 4, 3055-3064 (1977).

G. J. Hannon, Nature 418, 244-251 (2002).

G. J. Hannon et al., J Biol. Chem. 266, 22796-22799 (1991).

Hartwell L H, Mortimer R K, Culotti J, and Culotti M. (1973). Genetics, 74, 267-286.

Hock B, Bohme B, Kam T, Yamamoto T, Kaibuchi K, Holtrich U, Holland S, Pawson T, Rübsamen-Waigmann H, and Strebhardt K. (1998). Proc.Natl Acad Sci. U.S.A, 95, 9779-9784.

Holtrich U, Wolf G, Brauninger A, Karn T, Bohme B, Rubsamen-Waigmann H et al. Induction and downregulation of PLK, a human serine/threonine kinase expressed in proliferating cells and tumors. Proc Natl Acad Sci U S A 1994;91 :1736-1740.

Holtrich U, Wolf G, Yuan J, Bereiter-Hahn J, Kam T, Weiler M, Kauselmann G, Rehli M, Andreesen R, Kauftnann M, Kuhl D, and Strebhardt K. (2000). Oncogene, 19, 4832-4839.

Jang, Y.-J., Lin, C.-Y., Ma, S. and Erikson, R. L. Functional studies on the role of the C-terminal domain of mammalian polo-like kinase. Proc. Natl. Acad. Sci., USA, 99: 1984-1989, 2002

Kauselmann G, Weiler M, Wulff P, Jessberger S, Konietzko U, Scafidi J et al. The polo-like protein kinases Fnk and Snk associate with a Ca(2+)− and integrin-binding protein and are regulated dynamically with synaptic plasticity. EMBO J 1999; 18:5528-5539.

Kitada K, Johnson A L, Johnston L H, and Sugino A. (1993). Mol. Cell Biol., 13, 4445-4457.

Knecht R, Elez R, Oechler M, Solbach C, von llberg C, and Strebhardt K. (1999). Cancer Res., 59, 2794-2797.

Krieg, A. M., Matson, S., Fisher, E. 1996. Oligodeoxynucleotide modifications determine the magnitude of B cell stimulation by CpG motifs. Antisense Nucleic Acid Drug Dev. 6, 133-139.

Kuss B and Cotter F. (1999). Ann. Oncol., 10, 495-503.

Lake R J and Jelinek W R. (1993). Mol. Cell Biol., 13, 7793-7801.

Lane H A and Nigg E A. Antibody microinjection reveals an essential role for human polo-like kinase I (PLK1) in the functional maturation of mitotic centrosomes. J Cell Biol 1996; 135:1701-1713.

Latham, P. W. Therapeutic peptides revisited. Nat. Biotech., 17: 755-757, 1999.

Lee, K. S., Song, S. and Erikson, R. L. The polo-box-dependent induction of ectopic septal structures by mammalian polo kinase, Plk, in Saccharomyces cerevisiae. Proc. Natl. Acad. Sci., USA, 96:14360-14365, 1999.

D. L. Lewis, J, E. Hagstrom, A. G. Loomis, J. A. Wolff, H. Herweijer, Nat.Genet. 32, 107-108 (2002).

Li B, Ouyang B, Pan H, Reissmann P T, Slamon D J, Arceci R, Lu L, and Dai W. (1996).J.Biol. Chem., 271, 19402-19408.

Llamazares S, Moreira A, Tavares A, Girdharn C, Spruce B A, Gonzalez C, Karess R E, Glover D M, and Sunkel C E. (1991). Genes Dev., 5, 2153-2165.

S. M. Lobo, S. Ifill, N. Hernandez, Nucleic Acids Res. 18, 2891-2899 (1990).

May, K. M., Reynolds, N., Cullen, C. F., Yanagida, M. and Ohkura, H. Polo boxes and Cut23 (Apc8) mediate an interaction between polo kinase and the anaphase-promoting complex for fission yeast mitosis. J. Cell Biol., 156: 23-28, 2002.

A. P. McCaffrey et al., Nature 418, 38-39 (2002).

M. T. McManus and P. A. Sharp, Nat.Rev. Genet. 3, 737-747 (2002).

Monia B P, Sasmor H, Johnston J F, Freier S M, Lesnik E A, Muller M, Geiger T, Altmann K H, Moser H, and Fabbro D. (1996). Proc. Natl Acad Sci. U.S.A, 93, 15481-15484.

Montgomery M K, Xu S, and Fire A. RNA as a target of double-stranded RNA-mediated genetic interference in Caenorhabditis elegans. Proc Natl Acad Sci U S A 1998;95:15502-15507.

Morris, M C, Depollier, J, Mery, J, Heitz, Divita G. A peptide carrier for the delivery of biologically active protein into mammalian cells, Nat. Biotechnol. 2001, 19, 1173-6.

Mullin, J. M., Laughlin, K. V., Ginanni, N., Marano, C. W., Clarke, H. M., Peralta, S. A. 2000. Increased tight junction permeability can result from protein kinase C activation/translocation and act as a tumor promotional event in epithelial cancers. Ann. N. Y. Acad. Sci. 915, 231-236.

Mundt K E, Golsteyn R M, Lane H A, and Nigg E A. (1997). Biochem. Biophys. Res. Commun., 239, 377-385.

Mutoh, M., Lung, F. D., Long, Y. Q., Roller, P. P., Sikorski, R. S. and O'Connor, P. M. A p21^{Waf1/Cip1} carboxyl-terminal peptide exhibited cyclin-dependent kinase-inhibitory activity and cytotoxicity when introduced into human cells. Cancer Res., 59: 3480-3488, 1999.

Nemunaitis J, Holmiund J T, Kraynak M, Richards D, Bruce J, Ognoskie N, Kwoh T J, Geary R, Dorr A, Von Hoff D, and Eckhardt S G. (1999). J Clin. Oncol., 17, 3586-3595.

Nigg, E. A. Polo-like kinases: positive regulators of cell division from start to finish. Curr. Opin. Cell Biol., 10:776-783, 1998.

Nykanen, A., Haley, B., Zamore, P. D. 2001. ATP requirements and small interfering RNA structure in the RNA interference pathway. Cell 107, 309-321.

O'Dwyer P J, Stevenson J P, Gallagher M, Cassella A, Vasilevskaya 1, Monia B P, Holn-Aund J, Dorr F A, and Yao K S. (1999). Clin. Cancer Res., 5, 3977-3982.

Ohkura H, Hagan I M, and Glover D M. The conserved Schizosaccharomyces pombe kinase plo I, required to form a bipolar spindle, the actin ring, and septum, can drive septum formation in G 1 and G2 cells. Genes Dev 1995;9:1059-1073.

C. P. Paul, P. D. Good, 1. Winer, D. R. Engelke, Nat.Biotechnol. 20, 505-508 (2002).

Pihan G A, Purohit A, Wallace J, Malhotra R, Liotta L, and Doxsey S J. Centrosome defects can account for cellular and genetic changes that characterize prostate cancer progression. Cancer Res

Qian Y W, Erikson E, Taieb F E, and Mailer J L. The polo-like kinase Plx I is required for activation of the phosphatase Cdc25C and cyclin B-Cdc2 in Xenopus oocytes. Mol Biol Cell 2001;12:1791-1799.

Sharon G and Simchen G. (1990). Genetics, 125, 475-485.

Sharp P A. RNA interference—2001. Genes Dev 2001; 15:485-490.

Shirayama M, Zachariae W, Ciosk R, and Nasmyth K. The Polo-like kinase Cdc5p and the WD-repeat protein Cdc2Op/fizzy are regulators and substrates of the anaphase promoting complex in Saccharomyces cerevisiae. EMBO J 1998;17:1336-1349.

Simizu S and Osada H. Mutations in the Pik gene lead to instability of Pik protein in human tumour cell lines. Nat Cell Biol 2000;2:852-854.

Simmons D L, Neel B G, Stevens R, Evett G, and Erikson R L. (1992). Mol. Cell Biol., 12, 4164-4169.

Smith, M. R., Wilson, M. L., Hamanaka, R., Chase, D., Kung, H., Longo, D. L. and Ferris, D. K. Malignant transformation of mammalian cells initiated by constitutive expression of the polo-like kinase. Biochem. Biophys. Res. Commun., 234: 397-405, 1997.

Smits, V. A., Klompmaker, R., Arnaud, L., Rijksen, G., Nigg, E. A. and Medema, R. H. Polo-like kinase-1 is a target of the DNA damage checkpoint. Nat. Cell Biol., 2: 672-676, 2000.

B. Spänkuch-Schmitt, J. Bereiter-Hahn, M. Kaufmann, K. Strebhardt, J Natl. Cancer Inst. 94, 1863-1877 (2002).

Stein CA. (1995). Nat. Med, 1, 1119-1121.

Strebhardt K, Kneisel L, Linhart C, Bernd A, and Kaufmann R. (2000). JAMA, 283, 479-480.

Strebhardt, K. 2001. PLK (polo-like kinase). In Encyclopedia of Molecular Medicine. T. E. Creighton, editor. Wiley and Sons, Inc., New York, N.Y. 2530-2532.

G. Sui et al., Proc.Natl.Acad.Sci. U.S.A 99, 5515-5520 (2002).

Sunkel C E and Glover D M. (1988). J. Cell Sci., 89 (Pt 1), 25-38.

Tamm I, Dorken B, and Hartmann G. Antisense therapy in oncology: new hope for an old idea? Lancet 2001;358:489-497.

Toczyski, D. P., Galgoczy, D. J. and Hartwell, L. H. CDC5 and CKII control adaptation to the yeast DNA damage checkpoint. Cell, 90: 1097-2106, 1997.

Tokumitsu Y, Mori M, Tanaka S, Akazawa K, Nakano S, and Niho Y. (1999). Int. J Oncol., 15, 687-692.

Toyoshima-Morimoto F, Taniguchi E, Shinya N, lwamatsu A, and Nishida E. Polo-like kinase I phosphorylates cyclin B I and targets it to the nucleus during prophase. Nature 2001;410:215-220.

T. Tuschl, Nat.Biotechnol. 20, 446-448 (2002).

S. A. Vorburger and K. K. Hunt, Oncologist. 7, 46-59 (2002).

Wagner R W. (1995). Nat. Med, 1, 1116-1118.

Waters J S, Webb A, Cunningham D, Clarke P A, Raynaud F, di Stefano F, and Cotter F E. (2000). J Clin. Oncol., 18, 1812-1823.

Wolf G, Elez R, Doermer A, Holtrich U, Ackermann H, Stutte H J, Altmannsberger H M, Rübsamen-Waigmann H, and Strebhardt K. (1997). Oncogene, 14, 543-549.

H. Xia, Q. Mao, H. L. Paulson, B. L. Davidson, Nat.Biotechnol. 20,1006-1010 (2002).

Xie, S., Wu, H., Wang, Q., Cogswell, J. P., Husain, I., Conn, C., Stambrook, P., Jhanwar-Uniyal, M. and Dai, W. Plk3 functionally links DNA damage to cell cycle arrest and apoptosis at least in part via the p53 pathway. J. Biol. Chem., 276: 43305-43312, 2001;61:2212-2219.

Y. T. Yu, P. A. Maroney, E. Darzynkiwicz, T. W. Nilsen, RNA. 1, 46-54 (1995).

Yuan J, Horlin A, Hock B, Stutte H J, Rübsamen-Waigmann H, and Strebhardt K. Polo-like kinase, a novel marker for cellular proliferation. Am J Pathol 1997; 150:1165-1172.

Yuen A R, Halsey J, Fisher G A, HoIrnlund J T, Geary R S, Kwoh T J, Dorr A, and Sikic B I. (1999). Clin.Cancer Res., 5 3357-3363.

Zamore P D, Tuschl T, Sharp P A, and Bartel D P. RNAi: double-stranded RNA directs the ATP-dependent cleavage of mRNA at 21 to 23 nucleotide intervals. Cell 2000;101:25-33.

Zhang R, Yan J, Shahinian H, Amin G, Lu Z, Liu T, Saag M S, Jiang Z, Temsamani J, Martin R R, and. (1995). Clin. Pharmacol. Ther., 58, 44-53.

TABLE I

| | | Lage in der |
|---|---|---|
| Oligo- | | PLK1- bzw |
| Nummer | Sequenz | HSV-mRNA |
| P1 | cctccccggcaactctcgag | 5′-UTR |
| P2 | cgcaccgctccgctcctccc | 5′-UTR |
| P3 | gcagagcctccgcaccgctc | 5′-UTR |
| P4 | gcagacctcgatccgagcag | 5′-UTR |
| P5 | catgctcccgaagctgcgct | 5′-UTR |
| P6 | gtcactgcagcactcatgct | offener Leseraster |
| P7 | gtgccagcttccctgcagtc | offener Leseraster |
| P8 | cccagggtcggccggtgccc | offener Leseraster |
| P9 | tgtacaaaaataacttatac | offener Leseraster |
| P10 | cgggatctctttcgccggtg | offener Leseraster |
| P11 | gagggcagctattaggaggc | offener Leseraster |
| P12 | accagtccggaggggagggc | 3′-UTR |
| P13 | gggccccagatgcaggtggg | 3′-UTR |
| P14 | gcgggagccaaccagtatgg | 3′-UTR |
| P15 | ctgcagocatggcaccgcgg | 3′-UTR |
| P16 | caccggggctggggggccca | 3′-UTR |
| P17 | tgcagctctgcccagccacc | 3′-UTR |
| P18 | cccacctgcaaggatgatgc | 3′-UTR |
| P19 | tgtacaaaaataacttatac | 3′-UTR |
| P20 | ggacaaggctgtagaaccca | 3′-UTR |
| P21 | ggtggggttgagggggaggg | 3′-UTR |
| P22 | gaaatattctgtacaattca | 3′-UTR |
| P23 | aagctgcgctgcagacctcg | 5′-UTR |
| P24 | aagctgacttgcagacctcg | 5′-UTR |
| P25 | aagctgactgtgagacctcg | 5′-UTR |
| P26 | aagctgactgtgagacctta | 5′-UTR |
| HSV | gcggaggtccatgtcgtacgc | offener Leseraster |

1. Agent for inhibiting development or progress of proliferative diseases and especially cancer diseases or of other diseases which are accompanied by elevated PLK1 expression levels, characterized in that it reduces or inhibits the activity of polo like kinase 1 (PLK 1) in mammalian cells.

2. Agent according to claim 1, characterized in that it contains at least one short interfering RNA (siRNA) or antisense RNA which is directed against the PLK 1 gene as active agent.

3. Agent according to claim 2, characterized in that the RNA comprises 15 to 30 nucleotides.

4. Agent according to claim 2, characterized in that the sequence of the dsRNA or antisense RNA corresponds to nucleotide sequences of the PLK 1 mRNA.

5. Agent according to claim 4, characterized in that the dsRNA corresponds to positions 178-200 (siRNA2), 362-384 (siRNA3),1416-1438 (siRNA4) or 1570-1592 (siRNA5) of the PLK 1 gene.

6. Agent according to claim 1, comprising an effective amount of
1) at least one RNA expression system and optionally
2) a nuclease inhibiting substance, wherein said RNA expression system contains
a) at least one RNA polymerase specific promoter sequence and is under the transcriptional control of said promoter sequence
b) at least one genetic information homologous to the PLK 1 gene, wherein said genetic information under suitable conditions and in the presence of an RNA polymerase is transcribed into interfering RNA.

7. Agent according to claim 6, wherein said interfering RNA is a siRNA, preferably a shRNA (hairpin) or a short antisense RNA.

8. Agent according to claim 6, wherein said RNA expression system is contained in a plasmid or viral vector.

9. Agent according to claim 6, wherein the genetic information comprises two complementary and inverted sequences (hairpin) which are homologous to the PLK 1 gene.

10. Agent according to claim 9, wherein each of said two sequences is 15 to 30 nucleotides long.

11. Agent according to claim 9, wherein said sequences are connected by a spacer sequence.

12. Agent according to claim 11, wherein the spacer sequence contains 3 to 10 nucleotides.

13. Agent according to claim 6, wherein the genetic information b) contains an RNA polymerase stop signal at the 3′ end.

14. Agent according to claim 6, wherein the nuclease inhibitor is aurin tricarboxylic acid (ATA).

15. Agent according to claim 6, wherein the RNA specific promoter is the U6 promoter or H 1 promoter.

16. Agent according to claim 6, wherein it is formulated for intravenous administrations.

17. Agent according to claim 16, wherein it is formulated for bolus injection.

18. Agent according to claim 16, wherein the active substances are contained in buffered saline solution.

19. Agent according to claim 6, wherein the expression system is contained in an amount suitable for delivery of 0.05 to 0.5 mg/kg body weight of a patient.

20. Agent according to claim 1, characterized in that it contains at least one phosphorothiate antisense oligonucleotide (ASO) or an ASO with another modification like mixed backbone oligonucleotides or morpholino oligonucleotides directed against the PLK 1 gene as active agent.

21. Agent according to claim 20, characterized in that the ASO contains 15 to 30 nucleotides.

22. Agent according to claim 20, characterized in that the ASO is homologous to the PLK1 mRNA.

23. Agent according to claim 22, characterized in that the ASO is P12 and/or P13.

24. Agent according to claim 1, characterized in that a peptide which is inhibitory for the PLK 1 gene is present as active agent.

25. Agent according to claim 24, characterized in that the peptide comprises 3 to 50 amino acids.

26. Agent according to claim 24, characterized in that the peptide corresponds to a wild type (aa 410-439 in PLK1) or a mutated polo box or its polo-box similar structures in PLK 1-3.

27. Agent according to claim 24, characterized in that the peptide corresponds to the polo box or the mutated polo box with any modifications, like L-forward, L-reverse, D-reverse (retro-inverso), sidechain and backbone modifications, cyclic forms and repeats as well as other modifications which enhance the half-life of peptides.

28. Agent according to claim 24, characterized in that the peptide is linked to a protein transduction domain or is used together with a protein transduction domain without need for a chemical covalent coupling or other expression-vector systems (plasmids, viral vectors etc.).

29. Agent according to claim 24, characterized in that it contains peptide P1 and/or peptide P2.

30. Pharmaceutical composition, characterized by containing an effective amount of an agent according to claim 1, optionally together with useful auxiliary and/or carrier substances and/or inhibitors of proteinases.

31. Method for treating patients who suffer from proliferative disease and especially cancer disease, characterized by administration of an effective amount of an agent according to claim 1.
